(19) United States
(12) Patent Application Publication
(10) Pub. No.: US 2022/0267856 A1
(43) Pub. Date: Aug. 25, 2022

(54) Internal Standard Gene

(71) Applicant: Public University Corporation Fukushima Medical University, Fukushima (JP)

(72) Inventors: Shinya Watanabe, Fukushima (JP); Junichi Imai, Fukushima (JP); Emi Ito, Fukushima (JP); Toru Otake, Fukushima (JP); Noriko Abe, Fukushima (JP); Kazunoshin Tachibana, Fukushima (JP)

(21) Appl. No.: 17/627,678

(22) PCT Filed: Jul. 16, 2020
(86) PCT No.: PCT/JP2020/027650

§ 371 Date: Jan. 16, 2022

(30) Foreign Application Priority Data
Jul. 19, 2019 (JP) 2019-134182

(51) Int. Cl. C12Q 1/6886

(52) U.S. Cl. CPC C12Q 1/6886; C12Q 2600/112; C12Q 2600/166; C12Q 2600/158

(57) ABSTRACT

An object of the present invention is to provide a novel internal standard gene for gene expression analysis and to provide a gene expression analysis method using the internal standard gene. The present invention provides a gene expression analysis method for a test sample, comprising the steps of: (a) measuring an expression level of a desired gene; (b) measuring at least one internal standard gene selected from the group consisting of ABCF3, FBXW5, MLLT1, FAM234A, PITPNM1, WDR1, NDUFS7, and AP2A1; and (c) comparing the expression level of the desired gene with the expression level of the internal standard gene.

### TECHNICAL FIELD

The present invention relates to an internal standard gene. Particularly, the present invention relates to an internal standard gene that is used in measuring the expression quantity or expression level of a gene of interest present in a biological sample (including a breast cancer tissue and a normal mammary gland tissue) derived from a breast cancer patient.

### BACKGROUND ART

Gene expression analysis is one of the approaches of comparing two or more different biological samples and detecting their difference. The gene expression analysis involves extracting RNA contained in each biological sample and measuring the expression quantity or expression level of a particular gene therefrom for comparison. In the case of performing gene expression analysis by Northern blot, RT-PCR, real-time PCR, or the like, the comparison of the expression quantity or expression level of a particular gene is not sufficient and it is necessary to compare this expression quantity or expression level as a relative value to the expression quantity or expression level of a gene called internal standard gene. This is because, for example, (1) the amounts of biological samples to be compared are difficult to accurately adjust to the same amounts, and RNA extraction efficiency differs among biological samples even if the biological samples are used in the same amounts; and (2) the ratio of mRNA contained in RNA differs among biological samples even if RNA levels are the same. Thus, relative comparison with the internal standard gene has heretofore been required. For example, glyceraldehyde-3-phosphate dehydrogenase (GAPDH) gene and β actin (ACTB) gene known as so-called housekeeping genes have been traditionally used as the internal standard gene. These housekeeping genes are constitutively expressed in order to maintain the vital activities of cells themselves, and expressed at a constant level, irrespective of the type of cells or tissues. Therefore, the housekeeping genes are used on the assumption that they are not changed by experimental treatment.

However, the fact has become known that the expression of the internal standard gene differs depending on the type of cells or tissues and is adjusted by experimental conditions, the stages of development, etc. In actuality, there is a risk of misinterpretation ascribable to a selected internal standard gene whose expression quantity may vary depending on experimental conditions, etc., for gene expression analysis. Many researchers have also pointed out concerns about this. An internal standard gene that can be used universally, irrespective of particular tissues or experimental conditions, and usefully in normalization for gene expression analysis has already been searched for (Patent Literature 1). In such studies, however, search has often been made using gene expression data registered in an existing public database. Furthermore, such a public database includes the admixture of data measured under different platforms in a plurality of different facilities. Therefore, simple parallel comparison is actually difficult. On the other hand, there is also a study to search for a proper internal standard gene in a closed system dedicated to a particular experiment (Patent Literature 2). The internal standard gene thus searched for is selected in many cases as a result of verification based on data measured under the same platform in the same facility, and is therefore highly reliable as long as the internal standard gene is used in the system.

Breast cancer is variable and is classified into a plurality of subtypes having various features. This subtype classification was originally made by exhaustive gene expression analysis (Non Patent Literature 1). Since prognosis or drug sensitivity differs depending on this classification, this classification serves as an index for selecting medication. In actual clinical practice, diagnosis is generally performed using a convenient immunohistochemical approach, though not a few cases have breast cancer different from classification by gene expression analysis. Thus, there is a demand for highly accurate examination techniques by gene expression analysis. Research on breast cancer has a long history, and a large number of studies have been conducted by gene expression analysis using cell lines derived from breast cancer. Internal standard genes support the reliability of such examination techniques or studies.

### CITATION LIST

### Patent Literature

### [Patent Literature 1] Japanese Patent No. 5934036

### [Patent Literature 2] Japanese Patent Laid-Open No. 2012-105614

### Non Patent Literature

[Non Patent Literature 3] Perou C M, Sorlie T, Eisen M B, et al., Molecular portraits of human breast tumours. Nature 406: 747-752, 2000

### SUMMARY OF INVENTION

### Technical Problem

An object of the present invention is to provide a novel internal standard gene for gene expression analysis and to provide a gene expression analysis method using the internal standard gene. Particularly, an object of the present invention is to provide a gene capable of serving as an internal standard gene in the comparative analysis of samples derived from breast cancer, and a gene expression analysis method for a breast cancer-derived sample using the gene.

### Solution to Problem

In order to attain the object, the present inventors have obtained gene expression profiles of 14,400 genes from each of specimens of 470 cases in total involving breast cancer tissues (453 cases) and normal mammary gland tissues (17 cases), and successfully selected internal standard genes for the gene expression analysis of living tissues (including normal mammary gland tissues) derived from breast cancer, leading to the present invention.

Specifically, the present invention includes the following aspects.

In one aspect, the present invention relates to

[1] a gene expression analysis method for a test sample, comprising the steps of:

(a) measuring an expression level of a desired gene;

(b) measuring at least one internal standard gene selected from the group consisting of ABCF3, FBXW5, MLLT1, FAM234A, PITPNM1, WDR1, NDUFS7, and AP2A1; and

(c) normalizing the expression level of the desired gene using the expression level of the internal standard gene.

In this context, in one embodiment, the gene expression analysis method of the present invention is

[2] the gene expression analysis method according to [1], wherein

the test sample is a sample derived from a breast cancer patient.

In one embodiment, the gene expression analysis method of the present invention is

[3] the gene expression analysis method according to [2], wherein

the desired gene is a gene for identifying or classifying a subtype of breast cancer.

In another aspect, the present invention relates to

[4] an internal standard gene for gene expression analysis consisting of at least one gene selected from the group consisting of FBXW5, PITPNM1, MLLT1, WDR1, ABCF3, NDUFS7, FAM234A, and AP2A1.

In this context, in one embodiment, the internal standard gene of the present invention is

[5] the internal standard gene according to [4], wherein

the internal standard gene is used in gene expression analysis for a test sample derived from a breast cancer patient.

In one embodiment, the internal standard gene of the present invention is

[6] the internal standard gene according to [5], wherein

the gene expression analysis for a test sample derived from a breast cancer patient is gene expression analysis for identifying a subtype of breast cancer.

In an alternative aspect, the present invention relates to

[7] a composition for expression analysis of an internal standard gene, comprising a unit for measuring an expression level of an internal standard gene according to [4].

In this context, in one embodiment, the composition for expression analysis of an internal standard gene of the present invention relates to

[8] a composition for expression analysis of an internal standard gene for identifying or classifying breast cancer, comprising a unit for measuring an expression level of an internal standard gene according to [5] or [6].

In this context, in one embodiment, the composition for expression analysis of an internal standard gene for identifying or classifying breast cancer according to the present invention is

[9] the composition for expression analysis of an internal standard gene for identifying or classifying breast cancer according to [8], wherein

the unit for measuring an expression level of the gene is at least one unit selected from the group consisting of a primer, a probe, and an antibody against the gene, and labeled forms thereof.

In one embodiment, the composition for expression analysis of an internal standard gene for identifying or classifying breast cancer according to the present invention is

[10] the composition for expression analysis of an internal standard gene for identifying or classifying breast cancer according to [8] or [9], wherein

the composition is intended for PCR, a microarray, or RNA sequencing.

### Advantageous Effects of Invention

The internal standard gene of the present invention provides a novel internal standard gene that can be used in gene expression analysis. A gene expression analysis method using the internal standard gene can also be provided. The internal standard gene of the present invention substantially rarely varies in expression level, without being influenced by the subtype of breast cancer in a sample derived from a breast cancer patient, and differs only slightly in expression level relative to the expression level of a human common reference. Accordingly, the internal standard gene of the present invention is useful, particularly, in the gene expression analysis of a sample derived from breast cancer.

### BRIEF DESCRIPTION OF DRAWINGS

FIG. 1 is a graph showing the distributions of expression levels of four genes (ABCF3, MLLT1, FBXW5, and FAM234A) in the gene expression profiles of 470 samples measured in Example 3 described below.

FIG. 2 is a graph showing the distributions of expression levels of four genes (PITPNM1, NDUFS7, WDR1, and AP2A1) in the gene expression profiles of 470 samples measured in Example 3 described below.

FIG. 3 is a graph showing the distribution of expression levels of GAPDH gene in the gene expression profiles of 470 samples measured in Example 3 described below.

FIG. 4 shows a heatmap of results of conducting the cluster analysis of a gene averaging technique based on a Euclidean distance as to 470 cases using a set of 207 identification marker genes including internal standard genes shown in Example 4 described below.

### DESCRIPTION OF EMBODIMENTS

### 1. Internal Standard Gene

### 1-1. Summary

The first aspect of the present invention is an internal standard gene that can be used in gene expression analysis.

The internal standard gene according to the present invention consists of at least one gene selected from the group consisting of FBXW5, PITPNM1, MLLT1, WDR1, ABCF3, NDUFS7, FAM234A, and AP2A1.

### 1-2. Definition

The “internal standard gene” is a gene that is used in normalization for relatively indicating the amount of a particular gene in gene expression analysis. The internal standard gene according to the present invention is eight genes, FBXW5, PITPNM1, MLLT1, WDR1, ABCF3, NDUFS7, FAM234A, and AP2A1, as described above. The following table shows the symbols, gene names, and reference sequence IDs (RefSeq IDs) registered in the NCBI database, of these internal standard genes.

TABLE 1

| Symbol | Name | ID | SEQ ID NO |
|---|---|---|---|
| FBXW5 | F-box anal WD-40 domain protein 5 (FBXW5). trarizcript variant 2, mRNA. | NM_018998 | SEQ ID NO: 1 |
| PITPNM1 | phosphatidylinositol transfer protein membrane-associated 1 (PITPNM1), mRNA. | NM_004910 | SEQ ID NO: 2 |
| MILT1 | myeloid/lymphoid or mixed-lineage leukemia (trithorax homolog. Drosophila): translocated to, | NM_005934 | SEQ ID NO: 3 |
| | 1 (MILT1), mRNA. | | |
| WDR1 | WD repeat domain 1 (WDR1), transcript variant 1, mRNA. | NM_017491 | SEQ ID NO: 4 |
| ABCF3 | ATP-binding cassette, sub-family F (GCN20), member 3 (ABCF3), mRNA. | NM_018358 | SEQ ID NO: 5 |
| NDUFS7 | NADH dehydrogenase (ubiquinone) Fe—S protein 7, 20 kDa (NADH-coenzyme Q reductase) | NM_024407 | SEQ ID NO: 6 |
| | (NDUFS7), mRNA. | | |
| FAM234A | hypothetical protein DKFZp761D0211 (DKFZP761D0211), mRNA. | NM_032039 | SEQ ID NO: 7 |
| AP2A1 | adaptor-related protein complex 2, alpha 1 subunit (AP2A1). transcript variant 2, mRNA. | NM_130787 | SEQ ID NO: 8 |

The internal standard gene according to the present invention (FBXW5, PITPNM1, MLLT1, WDR1, ABCF3, NDUFS7, FAM234A, and AP2A1) can be defined as a gene (or a polynucleotide) having the nucleotide sequence represented by each of SEQ ID NOs: 1 to 8.

In one embodiment, the internal standard gene according to the present invention is at least one gene selected from the group consisting of a gene consisting of the nucleotide sequence represented by SEQ ID NO: 1, a gene consisting of the nucleotide sequence represented by SEQ ID NO: 2, a gene consisting of the nucleotide sequence represented by SEQ ID NO: 3, a gene consisting of the nucleotide sequence represented by SEQ ID NO: 4, a gene consisting of the nucleotide sequence represented by SEQ ID NO: 5, a gene consisting of the nucleotide sequence represented by SEQ ID NO: 6, a gene consisting of the nucleotide sequence represented by SEQ ID NO: 7, and a gene consisting of the nucleotide sequence represented by SEQ ID NO: 8.

In the present specification, the FBXW5, PITPNM1, MLLT1, WDR1, ABCF3, NDUFS7, FAM234A, and AP2A1 genes also include, for example, genes consisting of a nucleotide sequence containing a degenerate codon and encoding the same amino acid sequence, mutated genes, such as various variants and point mutant genes, of each gene, and ortholog genes of organisms of other species such as chimpanzees. Such genes include genes consisting of a polynucleotide consisting of a nucleotide sequence having 70% or higher (preferably 75% or higher, 80% or higher, or 85% or higher, more preferably 90% or higher, 95% or higher, 96% or higher, 97% or higher, 98% or higher, or 99% or higher) base identity to the nucleotide sequence defined in any of SEQ ID NOs: 1 to 8, the polynucleotide maintaining the functions of the intended gene.

In one embodiment, for example, the ABCF3 gene used in the present invention can be defined as a gene (or a polynucleotide) consisting of the nucleotide sequence represented by SEQ ID NO: 1. In this respect, the ABCF3 gene includes a gene consisting of a polynucleotide consisting of a nucleotide sequence having 70% or higher (preferably 75% or higher, 80% or higher, or 85% or higher, more preferably 90% or higher, 95% or higher, 96% or higher, 97% or higher, 98% or higher, or 99% or higher) base identity to the nucleotide sequence represented by SEQ ID NO: 1, the polynucleotide maintaining the functions of the ABCF3 gene. In the present specification, the “base identity” refers to the ratio (%) of the number of matched bases in the nucleotide sequences of nucleotides to be compared to the total number of bases in the genes when the two nucleotide sequences are aligned and a gap is introduced thereto, if necessary, so as to attain the highest degree of base matching between the nucleotide sequences.

In the present specification, the phrase “gene consisting of the nucleotide sequence represented by particular SEQ ID NO” also includes a polynucleotide hybridizing under stringent conditions to a nucleotide fragment consisting of a nucleotide sequence complementary to a partial nucleotide sequence of the gene, the polynucleotide maintaining the functions of the intended gene. The “stringent conditions” mean conditions under which any nonspecific hybrid is not formed. In general, more highly stringent conditions involve a lower salt concentration and a higher temperature. Low stringent conditions are, for example, conditions under which washing is performed with 1×SSC and 0.1% SDS at approximately 37° C. in washing after hybridization, and more stringent conditions are conditions under which washing is performed with 0.5×SSC and 0.1% SDS at approximately 42° C. to 50° C. Highly stringent conditions, which are much stricter, are, for example, conditions under which washing is performed with 0.1×SSC and 0.1% SDS at 50° C. to 70° C., 55° C. to 68° C., or 65° C. to 68° C. in washing after hybridization. In general, highly stringent conditions are preferred. The combinations of the SSC, the SDS and the temperature described above are mere illustrations. Those skilled in the art may determine the stringency of hybridization by appropriately combining the SSC, the SDS and the temperature as well as other conditions such as a probe concentration, a probe base length, and a hybridization time. In a preferred embodiment, the polynucleotide hybridizing under stringent conditions to a nucleotide fragment consisting of a nucleotide sequence complementary to a partial nucleotide sequence of a gene consisting of the nucleotide sequence represented by particular SEQ ID NO is a polynucleotide consisting of a nucleotide sequence having 70% or higher (preferably 75% or higher, 80% or higher, or 85% or higher, more preferably 90% or higher, 95% or higher, 96% or higher, 97% or higher, 98% or higher, or 99% or higher) base identity to the nucleotide sequence represented by the particular SEQ ID NO.

In the present specification, the term “internal standard gene” includes a gene defined by a DNA sequence as well as a gene product such as a transcript (mRNA and cDNA) and a translation product (protein) based on the gene.

At least one internal standard gene may be selected and used as the internal standard gene according to the present invention, or two or more internal standard genes may be used in combination. In the case of combining two or more internal standard genes, the internal standard genes may be selected from the group consisting of FBXW5, PITPNM1, MLLT1, WDR1, ABCF3, NDUFS7, FAM234A, and AP2A1, or any of these genes may be combined with an internal standard gene other than the group.

Examples of the internal standard gene other than the group consisting of FBXW5, PITPNM1, MLLT1, WDR1, ABCF3, NDUFS7, FAM234A, and AP2A1 can include commercially available universal references, housekeeping genes known in the art (e.g., glyceraldehyde-3-phosphate dehydrogenase (GAPDH) gene and actin (ACTB) gene) and combinations thereof. Those skilled in the art can select an internal standard gene suitable for conditions of each gene expression analysis through a test and can also use the selected internal standard gene together with the internal standard gene according to the present invention.

In one embodiment, the internal standard gene according to the present invention is an isolated gene (or gene product). In the present specification, the term “isolated” refers to a gene (or a gene product) isolated from a living body in the broad sense and includes a product obtained by substantially removing a factor naturally accompanying a gene (or a gene product) in the narrow sense.

The internal standard gene according to the present invention varies only slightly in the expression level of the gene among test samples derived from healthy individuals and patients having any subtype of breast cancer.

Accordingly, in one embodiment, the internal standard gene of the present invention can be used in gene expression analysis for a test sample derived from a breast cancer patient. In this context, the gene expression analysis for a test sample derived from a breast cancer patient includes, for example, gene expression analysis for identifying the presence or absence of breast cancer, and gene expression analysis for identifying or classifying a subtype of breast cancer. In this context, the term “identifying or classifying” refers to identifying the presence of breast cancer, identifying the high or low possibility that breast cancer is present, identifying or classifying any subtype to which breast cancer belongs, or identifying or classifying the high or low possibility that breast cancer belongs to any tissue type, as to a sample derived from a test subject having a history of breast cancer.

Use of the internal standard gene according to the present invention in the gene expression analysis for a test sample derived from a breast cancer patient enables a more accurate relative value of gene expression to be provided by comparison with an internal standard gene known in the art (e.g., GAPDH) or the like.

In the present specification, the “test sample” refers to a sample that is used in gene expression analysis. The test sample that can be used in the present invention is not particularly limited as long as the sample expresses the internal standard gene according to the present invention. Examples of the animal from which the test sample is derived can include humans, monkeys, and chimpanzees. A human is preferred. Examples of the “sample” can include tissue, cells, body fluids (blood (including serum, plasma and interstitial fluid), spinal fluid (cerebrospinal fluid), urine, lymph, digestive fluid, ascetic fluid, pleural effusion, fluid around the nerve root, extracts from each tissue or cell, etc.) and samples collected from living bodies, such as peritoneal lavages, cultured cells, and purified or prepared products thereof.

In the case of using the internal standard gene according to the present invention in the identification of breast cancer by gene expression analysis, the “test sample” is a sample collected from a human test subject and preferably contains a breast cancer tissue or a tissue suspected of being a breast cancer tissue, or a portion thereof. In this context, the “tissue” and the “cell” may be derived from any site of a test subject and is preferably a specimen collected by biopsy or surgically excised, more specifically, a breast tissue or a breast cell. A breast cancer cell collected by biopsy or a breast cancer tissue or a breast cancer cell suspected of having breast cancer is particularly preferred. Such a tissue or a cell may be formalin-fixed paraffin embedded (FFPE).

The “breast cancer” refers to a cancer that usually develops from the breast duct or a tissue within the breast duct, such as the lobule. The breast cancer includes carcinoma and sarcoma and refers to every malignant tumor of a breast tissue. The breast cancer is a heterogeneous disease and is classified into a plurality of subtypes having various features.

Subtypes of breast cancer are mainly classified into 11 types, luminal A, luminal B (HER2-positive), luminal B (HER2-negative), HER2-positive, HER2-positive-like, triple negative, phyllodes tumor, squamous cell cancer, indeterminable, normal-like, and normal.

In this context, the term “luminal A” clinicopathologically refers to a case that satisfies all of 1) ER positivity and PgR negativity, 2) HER2 negativity, 3) a low level of Ki67, and 4) a low risk of recurrence in MEGA. In the present specification, the term also includes cases similar in gene expression profiles to most of cases clinicopathologically diagnosed with luminal A.

The clinicopathological diagnosis of subtypes mainly involves, but is not limited to, confirming the expression of ER, PgR, HER2, and Ki67 by immunohistological staining, and also includes the confirmation thereof by gene expression analysis.

The term “luminal B (HER2-positive)” clinicopathologically refers to an ER-positive and HER2-positive case. In the present specification, the term also includes cases similar in gene expression profiles to most of cases clinicopathologically diagnosed with luminal B (HER2-positive).

The term “luminal B (HER2-negative)” clinicopathologically refers to a case that falls into any of 1) ER positivity and HER2 negativity, 2) a high level of Ki67, and 3) PgR negativity or a low level of PgR, and 4) a high risk of recurrence in MEGA. In the present specification, the term also includes cases more highly expressing a cell cycle-related gene group than other cases among luminal A cases.

The term “HER2-positive” clinicopathologically refers to a HER2-positive, ER-negative and PgR-negative case. In the present specification, the term also includes cases similar in gene expression profiles to most of cases clinicopathologically diagnosed with HER2-positive.

The term “HER2-positive-like” refers to a case that is HER2-negative but is similar in the other gene expression profiles to most of cases clinicopathologically diagnosed with HER2-positive.

The term “triple negative” clinicopathologically refers to an ER-negative, PgR-negative and HER2-negative case. In the present specification, the term also includes cases similar in gene expression profiles to most of cases clinicopathologically diagnosed with triple negative.

The term “squamous cell cancer” is a cancer caused by the malignant proliferation of cells called epidermal keratinocytes present in the epidermis. In the present specification, the term refers to a cancer that originates in the mammary gland.

The term “phyllodes tumor” is clinicopathologically similar to mammary fibroadenoma and refers to a tumor having the rapid proliferation of fibrous stroma and breast duct epithelium with respect to fibrous tumor in which intralobular connective tissues of the mammary gland proliferate.

The term “indeterminable” refers to a case that is not similar in gene expression profiles to any of luminal A, luminal B (HER2-positive), luminal B (HER2-negative), HER2-positive, HER2-positive-like, triple negative, normal-like, normal, squamous cell cancer and phyllodes tumor.

The term “normal-like” refers to a case that is clinicopathologically diagnosed with “cancer” but is similar in gene expression profiles to normal mammary gland tissues.

The term “normal” refers to a normal tissue.

The internal standard gene according to the present invention can be suitably used in gene expression analysis for identifying the subtypes of breast cancer listed above.

### 2. Composition for Gene Expression Analysis

### 2-1. Summary

In another aspect, the present invention relates to a composition for expression analysis of an internal standard gene, comprising a unit for measuring an expression level of the internal standard gene.

### 2-2. Definition

In the present specification, the “expression level of a gene” refers to the amount of a transcript, expression intensity or expression frequency of the gene. In this context, the expression level of a gene may include not only the expression level of the wild-type gene of the gene but the expression level of a mutated gene such as a point mutant gene. The transcript that indicates the expression of the gene may also include variant transcripts such as splicing variants and fragments thereof. This is because even information based on such a mutated gene, a transcript, or a fragment thereof permits use as the internal standard gene according to the present invention. The expression level of a gene can be obtained as a measurement value by the measurement of the amount of a transcript, i.e., a mRNA level, a cDNA level, etc., of the gene. In a preferred embodiment, the measurement of the expression level of a gene is the measurement of mRNA.

In the present specification, the “unit for measuring an expression level” is a compound that can bind to a gene transcript, and is a compound that can indicate the presence or absence of the transcript or the amount of the transcript. In one embodiment, the unit for measuring an expression level is at least one compound selected from the group consisting of a primer and a probe against the gene to be measured, and labeled forms thereof.

The primer or the probe that can be used in the present invention is usually constituted by a natural nucleic acid such as DNA or RNA. Highly safe, easy-to-synthesize, and inexpensive DNA is particularly preferred. If necessary, the natural nucleic acid may be combined with a chemically modified nucleic acid or a pseudo nucleic acid. Examples of the chemically modified nucleic acid or the pseudo nucleic acid include PNA (peptide nucleic acid), LNA (Locked Nucleic Acid®), methyl phosphonate-type DNA, phosphorothioate-type DNA, and 2′-O-methyl-type RNA. The primer or the probe can be labeled or modified with a labeling material such as a fluorescent material and/or a quencher material, or a radioisotope (e.g., 32P, 33P, and 35S), or a modifying material such as biotin or (strept)avidin, or magnetic beads, and used as a labeled form. The labeling material is not limited, and a commercially available product can be used. For example, a fluorescent material such as FITC, Texas, Cy3, Cy5, Cy7, cyanine 3, cyanine 5, cyanine 7, FAM, HEX, VIC, fluorescamine or a derivative thereof, or rhodamine or a derivative thereof can be used. A quencher material such as AMRA, DABCYL, BHQ-1, BHQ-2, or BHQ-3 can be used. The labeling position of the labeling material in the primer or the probe can be appropriately determined according to the characteristics of the modifying material or intended use. In general, a 5′- or 3′-terminal portion is often modified. One primer or probe molecule may be labeled with one or more labeling materials. The labeling of a nucleotide with such a material can be performed by a method known in the art.

A nucleotide for use as the primer or the probe may be any nucleotide composed of the sense strand or antisense strand of the gene to be measured. In a preferred embodiment, the nucleotide for use as the primer or the probe is a primer or a probe for PCR, for a microarray, or for RNA sequencing.

The base length of the primer or the probe is not particularly limited as long as the expression level of the gene of interest can be measured. The probe has at least a 10-base length or more to the full length of the gene, preferably a 15-base length or more to the full length of the gene, more preferably a 30-base length or more to the full length of the gene, further preferably a 50-base length or more to the full length of the gene, for use in a hybridization technique mentioned later, and has a 10- to 200-base length, preferably a 20- to 150-base length, more preferably a 30- to 100-base length, for use in a microarray. In general, a longer probe elevates hybridization efficiency and enhances sensitivity. On the other hand, a shorter probe reduces sensitivity but rather elevates specificity. The primer may be 10 to 50 bp, preferably 15 to 30 bp each of a forward primer and a reverse primer.

In the case of measuring the expression level of the internal standard gene according to the present invention by a nucleic acid amplification technique or the like, for example, probes shown in SEQ ID NOs: 9 to 16 can be used for FBXW5, PITPNM1, MLLT1, WDR1, ABCF3, NDUFS7, FAM234A, and AP2A1, respectively (Table 2), though the probe is not limited thereto. The preparation of the primer or the probe for each approach of gene expression analysis is known to those skilled in the art, and the primer or the probe can be prepared in accordance with, for example, a method described in Greene & Sambrook, Molecular Cloning (2012) mentioned above. It is also possible to provide sequence information to a nucleic acid synthesis commissioned manufacturer, which in turn produces the primer or the probe on consignment.

TABLE 2

| Symbol | Sequence ID | Sequence of probe | SEQ ID NO: |
|---|---|---|---|
| FBXW5 | NM_018998 | ACCACTGGCTGCCTCACCTACTCCCCACACCAGATCGGCATCA | SEQ ID NO: 9 |
| | | AGCAGATCCTGCCACACCAGATGACCACGGCAGGGCC | |
| PITPNM1 | NM_004910 | CACTCCAGCCTCTTTCTGGAGGAGCTGGAGATGCTGGTGCCCT | SEQ ID NO: 10 |
| | | CAACACCCACCTCTACTAGCGGTGCCTTCTGGAAGGG | |
| MLLT1 | NM_005934 | ATCTGATCGAGGAGACTGGCCACTTCAATGTCACCAACACCAC | SEQ ID NO: 11 |
| | | CTTCGACTTCGACCTCTTCTCCCTGGACGAGACCACC | |
| WDR1 | NM_017491 | AGCCTGGCCTGGCTGGACGAGCACACGCTGGTCACGACCTCC | SEQ ID NO: 12 |
| | | CATGATGCCTCTGTCAAGGAGTGGACAATCACCTACTG | |
| ABCF3 | NM_018358 | TGACTATGCCCTGCCCCAACTTCTACATTCTGGATGAACCCAC | SEQ ID NO: 13 |
| | | AAACCACCTGGACATGGAGACCATTGAGGCTCTGGGC | |
| NDUFS7 | NM_024407 | ACTATTCCTACTCGGTGGTGAGGGGCTGCGACCGCATCGTGCC | SEQ ID NO: 14 |
| | | CGTGGACATCTACATCCCAGGCTGCCCACCTACGGCC | |
| FAM234A | NM_032039 | TGGCACCGACAGACAGATCCTGTTTCTGGACCTTGGCACTGGA | SEQ ID NO: 15 |
| | | GCCGTCCTGTGTAGCCTAGCCCTCCCGAGCCTCCCTG | |
| AP2A1 | NM_130787 | AGCATTCCAACGCCAAGAACGCCATCCTCTTCGAGACCATCAG | SEQ ID NO: 16 |
| | | CCTCATCATCCACTATGACAGTGAGCCCAACCTCCTG | |

When the unit for measuring an expression level is any of the probes as described above, each probe may be provided in the form of a DNA microarray or a DNA microchip in which the probe is immobilized on a substrate. The material of the substrate for immobilizing each probe thereon is not limited, and, for example, a glass plate, a quartz plate, or a silicon wafer is usually used. Examples of the size of the substrate include 3.5 mm×5.5 mm, 18 mm×18 mm, and 22 mm×75 mm, which can be variously set according to the number of probe spots, the size of the spots, etc. For the probe, 0.1 μg to 0.5 μg of a nucleotide is usually used per spot. Examples of the nucleotide immobilization method include a method of electrostatically binding the nucleotide through the use of its charge to a solid-phase support surface-treated with a polycation such as polylysine, poly-L-lysine, polyethyleneimine, or polyalkylamine, and a method of covalently binding the nucleotide harboring a functional group such as an amino group, an aldehyde group, a SH group, or biotin to solid-phase surface harboring a functional group such as an amino group, an aldehyde group, or an epoxy group.

The composition for expression analysis of an internal standard gene of the present invention may be provided as a kit comprising other reagents (e.g., probes or primers against other genes, or labeled forms thereof, or a buffer) or an instrument (culture dish, etc.) necessary for the measurement or detection of gene expression, and an instruction for use in the identification of breast cancer.

### 3. Gene Expression Analysis Method Using Internal Standard Gene

### 3-1. Summary

In an alternative aspect, the present invention relates to a gene expression analysis method for a test sample using the internal standard gene according to the present invention. The gene expression analysis method comprises the steps of:

(a) measuring an expression level of a desired gene;

(b) measuring at least one internal standard gene selected from the group consisting of ABCF3, FBXW5, MLLT1, FAM234A, PITPNM1, WDR1, NDUFS7, and AP2A1; and

(c) normalizing the expression level of the desired gene using the expression level of the internal standard gene.

### 3-2. Definition

The gene expression analysis method according to the present invention comprises the step of (a) measuring an expression level of the desired gene.

The “step of measuring an expression level of the gene” is the step of measuring an expression level of the gene in a test sample to obtain a measurement value thereof.

The measurement of the expression level of the gene is preferably the measurement of the expression level per unit amount. In the present specification, the “unit amount” refers to an arbitrarily set amount of a sample. For example, a volume (indicated by μL or mL) or a weight (indicated by μg, mg, or g) corresponds thereto. The unit amount is not particularly defined, and the unit amount to be measured by a series of procedures in the gene expression analysis method is preferably constant.

In the present specification, the “desired gene” refers to a gene whose relative value of a gene expression level is to be examined using the internal standard gene according to the present invention. The desired gene is not particularly limited as long as the gene is expressed in the same cells as those expressing the internal standard gene according to the present invention. Those skilled in the art can appropriately select the desired gene according to the purpose of gene expression analysis. In one embodiment, the desired gene is a gene for identifying breast cancer. The gene for identifying breast cancer is a gene whose expression level varies specifically in breast cancer. Accordingly, in the embodiment, the desired gene can be a gene known to specifically exhibit variations in expression level in breast cancer. In a more preferred embodiment, the desired gene is a gene for identifying or classifying a subtype of breast cancer.

Examples of the gene for identifying or classifying a subtype of breast cancer can include, but are not limited to, ABCF3 gene, FBXW5 gene, MLLT1 gene, FAM234A gene, PITPNM1 gene, WDR1 gene, NDUFS7 gene, AP2A1 gene, KRTDAP gene, SERPINB3 gene, SPRR2A gene, SPRR1B gene, KLK13 gene, KRT1 gene, LGALS7 gene, PI3 gene, SERPINH1 gene, SNAI2 gene, GPR173 gene, HAS2 gene, PTH1R gene, PAGE5 gene, ITLN1 gene, SH3PXD2B gene, TAP1 gene, FN1 gene, CTHRC1 gene, MMP9 gene, ADIPOQ gene, CD36 gene, GOS2 gene, GPD1 gene, LEP gene, LIPE gene, PLIN1 gene, SDPR gene, LIFR gene, TGFBR3 gene, CAPN6 gene, PIGR gene, KRT15 gene, KRT5 gene, KRT14 gene, DST gene, WIF1 gene, SYNM gene, KIT gene, GABRP gene, SFRP1 gene, ELF5 gene, MIA gene, MMP7 gene, FDCSP gene, CRABP1 gene, PROM1 gene, KRT23 gene, S100A1 gene, WIPF3 gene, CYYR1 gene, TFCP2L1 gene, DSC2 gene, MFGE8 gene, KLK7 gene, KLK5 gene, DSG3 gene, TTYH1 gene, SCRG1 gene, S100B gene, ETV6 gene, OGFRL1 gene, MELTF gene, HORMAD1 gene, PKP1 gene, FOXC1 gene, ITGB8 gene, VGLL1 gene, ART3 gene, EN1 gene, SPHK1 gene, TRIM47 gene, COL27A1 gene, RFLNA gene, RASD2 gene, A2ML1 gene, MARCO gene, TSPYL5 gene, TM4SF1 gene, FABP5 gene, SPIB gene, BCL2A1 gene, MZB1 gene, KCNK5 gene, LMO4 gene, RNF150 gene, LYZ gene, C21orf58 gene, ATP13A5 gene, NUDT8 gene, HSD17B2 gene, ABCA12 gene, ENPP3 gene, WNT5A gene, MPP3 gene, VPS13D gene, PXMP4 gene, GGT1 gene, TRPV6 gene, C2orf54 gene, CLDN8 gene, LBP gene, SRD5A3 gene, PAPSS2 gene, TMEM45B gene, CLCA2 gene, FASN gene, MPHOSPH6 gene, NXPH4 gene, HPGD gene, KYNU gene, GLYATL2 gene, KMO gene, SRPK3 gene, THRSP gene, PLA2G2A gene, TFAP2B gene, FABP7 gene, SLPI gene, SERHL2 gene, S100A9 gene, KRT7 gene, TMEM86A gene, MBOAT1 gene, PGAP3 gene, STARD3 gene, ERBB2 gene, MIEN1 gene, GRB7 gene, GSDMB gene, ORMDL3 gene, MED24 gene, MSL1 gene, CASC3 gene, WIPF2 gene, THSD4 gene, MAPT gene, LONRF2 gene, TCEAL3 gene, DBNDD2 gene, FGD3 gene, GFRA1 gene, PARD6B gene, STC2 gene, SLC39A6 gene, ENPP5 gene, ZNF703 gene, EVL gene, TBC1D9 gene, CHAD gene, GREB1 gene, HPN gene, IL6ST gene, FAM198B gene, CA12 gene, KCNE4 gene, NAT1 gene, CYP2B6(CYP2B7P) gene, ARMT1 gene, MAGED2 gene, CELSR1 gene, INPP5J gene, PADI2 gene, PPP1R1B gene, ESR1 gene, MLPH gene, FOXA1 gene, XBP1 gene, GATA3 gene, ZG16B gene, KIAA0040 gene, TMC4 gene, AGR2 gene, TFF3 gene, SCGB2A2 gene, MUCL1 gene, DDX11 gene, ATAD2 gene, GGH gene, CDCA3 gene, CCNA2 gene, CCNB2 gene, ANLN gene, UBE2C gene, CKS2 gene, MKI67 gene, FOXMl gene, UBE2T gene, MCM4 gene, CKAP2 gene, HNl gene, KPNA2 gene, H2AFX gene, H2AFZ gene, CDK1 gene, PTTG1 gene, CDCl20 gene, MYBL2 gene and RRM2 gene.

The measurement of gene expression can be carried out by the measurement of a transcript. Hereinafter, the method for measuring a gene transcript will be specifically described. The method for measuring a gene transcript is known in the art. The method will be described below with reference to or by citation of the description about a method for measuring a gene transcript or a translation product in Japanese Patent Laid-Open No. 2016-13081. Also, a typical method for measuring a gene transcript will be described below. However, the method is not limited thereto, and a measurement method known in the art can be used.

The measurement of a transcript of the identification gene may be the measurement of a mRNA level or may be the measurement of a cDNA level obtained by reverse transcription from mRNA. In general, the measurement of a gene transcript adopts a method of measuring the expression level of the gene as an absolute value or a relative value using a nucleotide primer or probe comprising the whole or a portion of the nucleotide sequence of the gene.

The measurement of a gene transcript can be a nucleic acid detection and/or quantification method known in the art and is not particularly limited. Examples thereof include hybridization techniques, nucleic acid amplification techniques and RNA sequencing (RNA-Seq) analysis techniques.

The “hybridization technique” is a method of using, as a probe, a nucleic acid fragment having a nucleotide sequence complementary to the whole or a portion of the nucleotide sequence of a target nucleic acid to be detected, and detecting or quantifying the target nucleic acid or a fragment thereof through the use of the base pairing between the nucleic acid and the probe. In this aspect, the target nucleic acid corresponds to mRNA or cDNA of each gene constituting an identification marker, or a fragment thereof. In general, the hybridization technique is preferably performed under stringent conditions in order to eliminate unintended nonspecifically hybridizing nucleic acids. The highly stringent conditions involving a low salt concentration and a high temperature as mentioned above is more preferred. Some methods that differ in detection approach are known as the hybridization technique. For example, Northern blot (Northern hybridization technique), a microarray technique, a surface plasmon resonance technique or a quartz crystal microbalance technique is suitable.

The “Northern blot” is the most general method for analyzing gene expression and is a method of separating total RNA or mRNA prepared from a sample by electrophoresis using agarose gel or polyacrylamide gel, etc. under denaturation conditions, and transferring (blotting) the product to a filter, followed by the detection of a target nucleic acid using a probe having a nucleotide sequence specific for the target RNA. The probe may be labeled with an appropriate marker such as a fluorescent dye or a radioisotope and thereby enables the target nucleic acid to be quantified using a measurement apparatus, for example, a chemiluminescence photographing and analysis apparatus (e.g., Light Capture; ATTO Corp.), a scintillation counter, or an imaging analyzer (e.g., FUJIFILM Corp.: BAS series). The Northern blot is a technique well known and prominent in the art. See, for example, Greene, M. R. and Sambrook, J. (2012) mentioned above.

The “microarray technique” is a method of arranging, on a substrate, small spots at a high density of a probe which is a nucleic acid fragment complementary to the whole or a portion of the nucleotide sequence of a target nucleic acid, reacting the resulting solid-phase microarray or microchip with a sample containing the target nucleic acid, and detecting a nucleic acid hybridized with the substrate spot through fluorescence or the like. The target nucleic acid may be RNA such as mRNA, or DNA such as cDNA. The detection or quantification can be achieved by detecting or measuring fluorescence or the like based on the hybridization of the target nucleic acid, etc. using a microplate reader or a scanner. The mRNA level or the cDNA level or an abundance ratio thereof to reference mRNA can be determined from the measured fluorescence intensity. The microarray technique is also a technique well known in the art. See, for example, a DNA microarray technique (DNA Microarray and Latest PCR Method (2000), Masaaki Muramatsu and Hiroyuki Nawa ed., Gakken Medical Shujunsha Co., Ltd.).

The “surface plasmon resonance (SPR) technique” is a method of very highly sensitively detecting or quantifying an adsorbed matter on the surface of a thin metal film through the use of a surface plasmon resonance phenomenon in which, as the incident angle of laser beam used to irradiate the thin metal film is changed, reflexed light intensity attenuates markedly at a particular incident angle (resonance angle). In the present invention, for example, a probe having a sequence complementary to the nucleotide sequence of a target nucleic acid is immobilized on the surface of the thin metal film, and the other surface portion of the thin metal film is subjected to blocking treatment. Then, a sample collected from a test subject or a healthy subject or a healthy subject group is distributed to the surface of the thin metal film so that the target nucleic acid and the probe form base pairing. The target nucleic acid can be detected or quantified from the difference in measurement value between before and after the samples distribution. The detection or quantification by the surface plasmon resonance technique can be performed using, for example, an SPR sensor commercially available from Biacore/Cytiva. This technique is well known in the art. See, for example, Kazuhiro Nagata and Hiroshi Handa, Real-Time Analysis of Biomolecular Interactions, Springer-Verlag Tokyo, Tokyo, Japan, 2000.

The “quartz crystal microbalance (QCM) technique” is mass spectrometry of quantitatively determining a very small amount of an adsorbed matter from the amount of change in resonance frequency through the use of a phenomenon in which, when a substance is adsorbed to the surface of an electrode attached to a quartz crystal unit, the resonance frequency of the quartz crystal unit is decreased according to the mass thereof. The detection or quantification by this method can also employ a commercially available QCM sensor, as in the SPR technique. For example, a probe having a sequence complementary to the nucleotide sequence of a target nucleic acid is immobilized on the surface of the electrode, and the target nucleic acid can be detected or quantified from the base pairing between the probe and the target nucleic acid in a sample collected from a test subject or a healthy subject or a healthy subject group. This technique is well known in the art. See, for example, Christopher J. et al., 2005, Self-Assembled Monolayers of a Form of Nanotechnology, Chemical Review, 105: 1103-1169 and Toyosaka Moriizumi and Takamichi Nakamoto, (1997) Sensor Engineering, Shokodo Co., Ltd.

The “nucleic acid amplification technique” is a method of amplifying a particular region of a target nucleic acid with nucleic acid polymerase using forward and reverse primers. Examples thereof include PCR (including RT-PCR), NASBA, ICAN, and LAMP® (including RT-LAMP). PCR is preferred. The method for measuring a gene transcript by use of the nucleic acid amplification technique employs a quantitative nucleic acid amplification technique such as real-time RT-PCR. Further, an intercalator technique using SYBR® Green or the like, a Taqman® probe technique, digital PCR, and a cycling probe technique are known as real-time RT-PCR, and any of the methods can be used. All of these methods are known in the art and are described in appropriate protocols in the art. See such protocols.

The “RNA sequencing (RNA-Seq) analysis technique” refers to a method of converting RNA to cDNA through reverse transcription reaction, and counting the number of reads thereof using a next-generation sequencer (e.g., which includes, but not limited to, HiSeq series (Illumina, Inc.) and Ion Proton System (Thermo Fisher Scientific Inc.)) to measure the expression quantity of the gene. All of these methods are known in the art and are described in appropriate protocols in the art. See such protocols.

Hereinafter, the method for quantifying a gene transcript by RT-PCR will be briefly described by taking one example. The real-time RT-PCR is a nucleic acid quantification method of performing PCR using a temperature cycler apparatus having a function of detecting fluorescence intensity derived from an amplification product in a reaction system in which cDNA prepared through reverse transcription reaction from mRNA in a sample is used as a template and the PCR amplification product is specifically fluorescently labeled. The amount of the amplification product from a target nucleic acid is monitored in real time during reaction, and the results are subjected to regression analysis in a computer. The method for labeling the amplification product includes a method using a fluorescently labeled probe (e.g., TaqMan® PCR) and an intercalator method using a reagent specifically binding to double-stranded DNA. The TaqMan® PCR employs a probe modified at its 5′-terminal portion with a quencher material and at its 3′-terminal portion with a fluorescent dye. The quencher material at the 5′-terminal portion usually inhibits the fluorescent dye at the 3′-terminal portion. Upon PCR, the probe is degraded by the 5′→3′ exonuclease activity of Taq polymerase, thereby canceling the inhibition of the quencher material. Therefore, fluorescence is emitted. The amount of the fluorescence reflects the amount of the amplification product. When the amplification product reaches a detection limit, the number of cycles (CT) is in inverse correlation with the initial amount of the template. Therefore, in the real-time measurement technique, the initial amount of the template is quantified by measuring CT. Provided that CT is measured using several known amounts of the template to prepare a calibration curve, the absolute value of the initial amount of the template in an unknown sample can be calculated. For example, M-MLV RTase, ExScript RTase (Takara Bio Inc.), or Super Script II RT (Thermo Fisher Scientific Inc.) can be used as reverse transcriptase for use in RT-PCR.

The reaction conditions of real-time PCR are generally based on PCR known in the art and vary depending on the base length of a nucleic acid fragment to be amplified and the amount of a templated nucleic acid as well as the base length and Tm value of the primer used, the optimum reaction temperature and optimum pH of the nucleic acid polymerase used, etc. Therefore, the reaction conditions can be appropriately determined according to these conditions. As one example, usually, denaturation reaction is performed at 94 to 95° C. for 5 seconds to 5 minutes; annealing reaction is performed at 50 to 70° C. for 10 seconds to 1 minute; and elongation reaction is performed at 68 to 72° C. for 30 seconds to 3 minutes. This cycle can be repetitively performed 15 to 40 times to perform the elongation reaction. In the case of using a kit commercially available from any of the manufacturers, this operation can be performed according to a protocol attached to the kit, as a rule.

The nucleic acid polymerase for use in real-time PCR is DNA polymerase, particularly, thermostable DNA polymerase. Such nucleic acid polymerase is commercially available as various types, which may be used. Examples thereof include Taq DNA polymerase attached to Applied Biosystems TaqMan MicroRNA Assays Kit (Thermo Fisher Scientific Inc.) described above. Particularly, such a commercially available kit is useful because a buffer or the like optimized for the activity of the attached DNA polymerase is attached to the kit.

The gene expression analysis method of the present invention comprises, in addition to the step (a), a step of (b) measuring at least one internal standard gene selected from the group consisting of ABCF3, FBXW5, MLLT1, FAM234A, PITPNM1, WDR1, NDUFS7, and AP2A1.

The step (a) and the step (b) can be performed at the same time or may be separately performed such that either of these steps may be performed first. In a preferred embodiment, the steps (a) and (b) are performed at the same time.

The gene expression analysis method of the present invention comprises, after the step (a) and the step (b), a step of (c) normalizing the expression level of the desired gene using the expression level of the internal standard gene.

In the present specification, the term “normalizing” refers to enabling the expression level of the desired gene measured under particular conditions in a test sample to be compared with the expression level of the desired gene measured under different conditions in the test sample. More specifically, the term means that the expression level of the desired gene measured under particular conditions in a test sample is compared with the expression level of the internal standard gene measured under the same conditions in the test sample, thereby calculating the expression level of the desired gene in the test sample as a relative value to the expression level of the internal standard gene.

In the normalization step, the method for calculating the expression level of the desired gene as a relative value is not limited as long as this expression level can be compared with the expression level of the desired gene measured under different conditions in the test sample. The expression level of the desired gene measured under particular conditions in a test sample can be indicated as a relative value, for example, by dividing its value by the value of the expression level of the internal standard gene measured under the same conditions in the test sample.

The expression level of the desired gene normalized by the step (c) can be relatively compared with the expression level of the gene measured under different conditions and normalized by a similar approach.

### EXAMPLES

### Example 1. RNA Preparation

Total RNA was extracted from surgically collected breast cancer tissues using ISOGEN (Nippon Gene Co., Ltd., Tokyo, Japan). Normal mammary gland tissues and some breast cancer tissues were purchased from an overseas agency, and total RNA was extracted therefrom in the same manner as above. Samples from which 125 μg or more of total RNA was able to be obtained were subsequently subjected to poly(A)+RNA purification using MicroPoly(A) purist Kit (Ambion, Austin, Tex., USA).

The human common reference RNA used was Human Universal Reference RNA Type I (MicroDiagnostic, Tokyo, Japan) or Human Universal Reference RNA Type II (MicroDiagnostic).

### Example 2. Exhaustive Gene Expression Analysis

The DNA microarray used in the obtainment of gene expression profiles using poly(A)+RNA (designated as “system 1”) was a glass slide on which 31,797 types of synthetic DNAs (80 mers) (MicroDiagnostic) corresponding to human-derived transcripts were arrayed using a custom arrayer. On the other hand, the DNA microarray used for the obtainment of gene expression profiles using total RNA (designated as “system 2”) was a glass slide on which 14,400 types of synthetic DNAs (80 mers) (MicroDiagnostic) corresponding to human-derived transcripts were arrayed using a custom arrayer.

As for the specimen-derived RNA, labeled cDNA was synthesized from 2 μg of poly(A)+RNA for the system 1 and from 5 μg of total RNA for the system 2 using SuperScript II (Invitrogen Life Technologies, Carlsbad, Calif., USA) and Cyanine 5-dUTP (Perkin-Elmer Inc.). Likewise, for the human common reference RNA, labeled cDNA was synthesized from 2 μg of poly(A)+RNA or 5 μg of total RNA using SuperScript II and Cyanine 3-dUTP (Perkin-Elmer Inc.).

Hybridization to the DNA microarray was performed using Labeling and Hybridization kit (MicroDiagnostic).

Fluorescence intensity after hybridization to the DNA microarray was measured using GenePix 4000B Scanner (Axon Instruments, Inc., Union city, CA, USA). An expression ratio was calculated by dividing the fluorescence intensity of the specimen-derived Cyanine-5-labeled cDNA by the fluorescence intensity of the human common reference-derived Cyanine-3-labeled cDNA (fluorescence intensity of the specimen-derived Cyanine-5-labeled cDNA/fluorescence intensity of the human common reference-derived Cyanine-3-labeled cDNA). Further, normalization was performed by multiplying the calculated expression ratio by a normalization factor using GenePix Pro 3.0 software (Axon Instruments, Inc.,). Next, the expression ratio was converted to log 2, and the converted value was designated as a log 2 ratio. The conversion of the expression ratio was performed using Excel software (Microsoft, Bellevue, Wash., USA) and MDI gene expression analysis software package (MicroDiagnostic).

### Example 3. Internal Standard Gene Useful in Identifying Breast Cancer Subtype

In this Example, genes whose expression pattern did not vary depending on any subtype of breast cancer were determined.

According to the RNA preparation method and the exhaustive gene expression analysis method described above in Examples 1 and 2, gene expression profiles of 14,400 genes were obtained as to each of specimens of 470 cases in total involving breast cancer tissues (453 cases) and normal mammary gland tissues (17 cases).

Among the obtained gene expression profiles, genes were picked up which substantially rarely varied in expression ratio depending on any subtype of breast cancer. Specifically, internal standard genes were selected from genes for which the number of specimens having no detectable signal was 3 or less; the absolute value of the expression ratio was less than 0.45; the standard deviation was less than 0.35; the value of maximum value−minimum value was less than 2.2; and the average value of “sum of medians” exceeded 400. As a result, eight genes, ABCF3, FBXW5, MLLT1, FAM234A, PITPNM1, WDR1, NDUFS7, and AP2A1, were successfully selected as internal standard genes.

The following table shows standard deviations, maximum values, and minimum values in the gene expression profiles of ABCF3, FBXW5, MLLT1, FAM234A, PITPNM1, WDR1, NDUFS7, and AP2A1.

TABLE 3

| Symbol | ABCF3 | FBXW5 | MLLT1 | FAM234A | PITPNM1 | WDR1 | NDUFS7 | AP2A1 |
|---|---|---|---|---|---|---|---|---|
| Standard deviation | 0.253 | 0.247 | 0.292 | 0.296 | 0.269 | 0.329 | 0.273 | 0.282 |
| Maximum value | 1.647 | 1.213 | 1.052 | 1.240 | 1.132 | 0.739 | 0.637 | 0.602 |
| Minimum value | −0.425 | −0.951 | −1.132 | −0.501 | −1.053 | −2.250 | −1.305 | −1.232 |

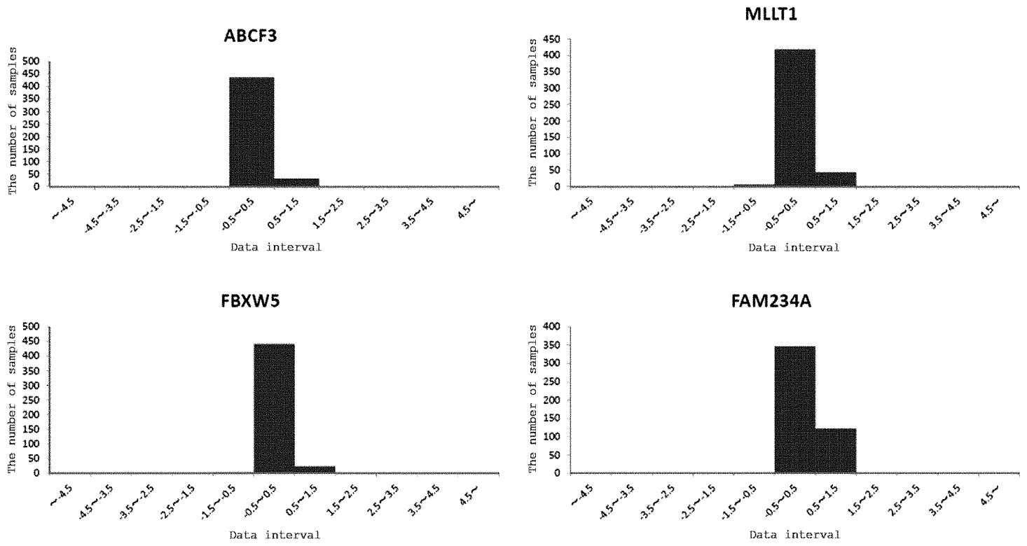

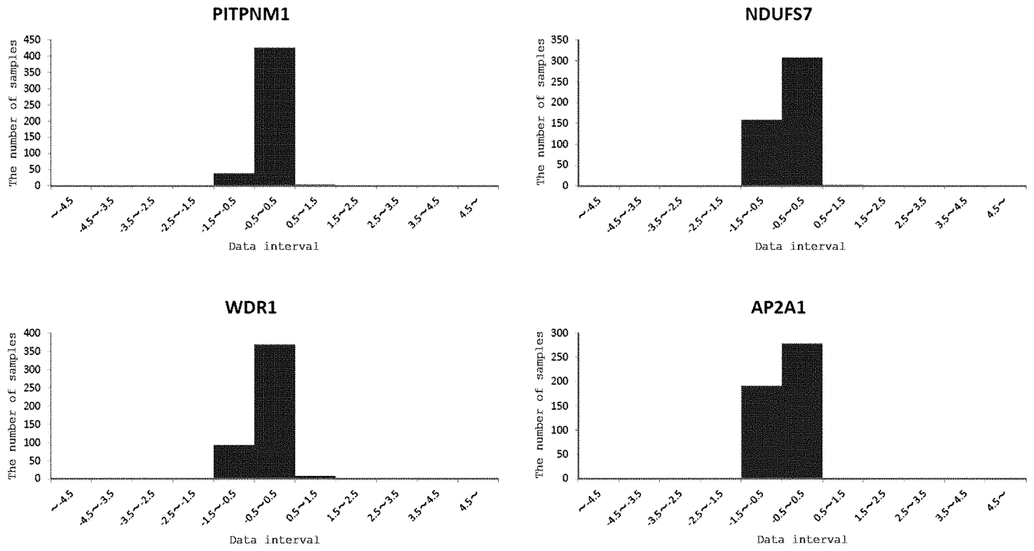

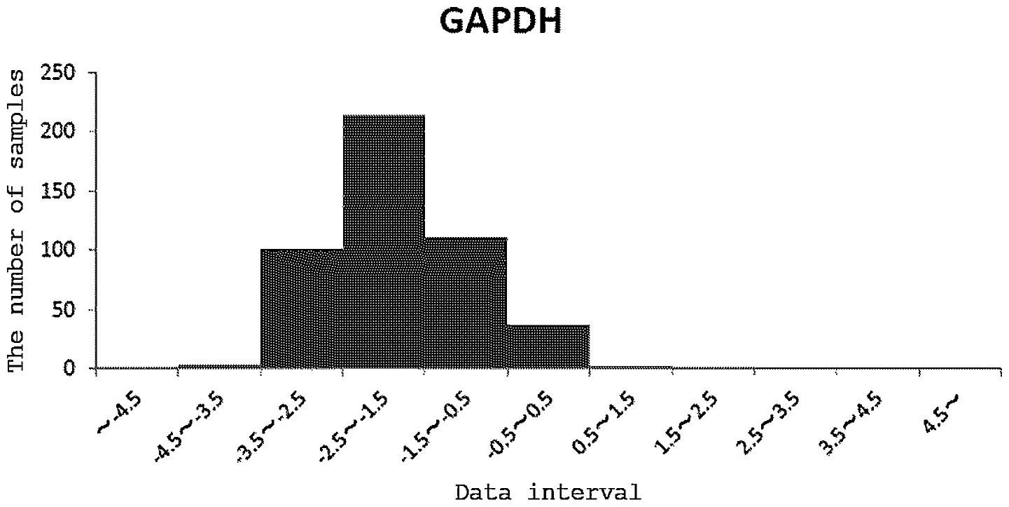

Table 4 given below and FIGS. 1 to 3 show a distribution at each data interval of the expression ratio delimited by 1.0 in the gene expression profiles of the eight internal standard genes. Table 4 and FIGS. 1 to 3 also show a distribution of GAPDH (Accession No: NM_002046) heretofore used as a housekeeping gene for the comparison of the eight internal standard genes.

TABLE 4

| Data | Frequency | | | | | | | | |
|---|---|---|---|---|---|---|---|---|---|
| interval | ABCF3 | FBXW5 | MLLT1 | FAM234A | PITPNM1 | WDR1 | NDUFS7 | AP2A1 | GAPDH |
| ~−4.5 | 0 | 0 | 0 | 0 | 0 | 0 | 0 | 0 | 1 |
| −4.5~−3.5 | 0 | 0 | 0 | 0 | 0 | 0 | 0 | 0 | 3 |
| −3.5~−2.5 | 0 | 0 | 0 | 0 | 0 | 0 | 0 | 0 | 101 |
| −2.5~−1.5 | 0 | 0 | 0 | 0 | 0 | 1 | 0 | 0 | 214 |
| −1.5~−0.5 | 0 | 4 | 7 | 1 | 39 | 93 | 159 | 191 | 111 |
| −0.5~0.5 | 437 | 442 | 419 | 347 | 427 | 368 | 308 | 278 | 37 |
| 0.5~1.5 | 32 | 24 | 44 | 122 | 4 | 8 | 3 | 1 | 2 |
| 1.5~2.5 | 1 | 0 | 0 | 0 | 0 | 0 | 0 | 0 | 1 |
| 2.5~3.5 | 0 | 0 | 0 | 0 | 0 | 0 | 0 | 0 | 0 |
| 3.5~4.5 | 0 | 0 | 0 | 0 | 0 | 0 | 0 | 0 | 0 |
| 4.5~ | 0 | 0 | 0 | 0 | 0 | 0 | 0 | 0 | 0 |

As shown in Table 4 and FIGS. 1 to 3, the eight internal standard genes less varied in expression ratio among samples than the GAPDH gene. Most of distributions of the expression ratio fell within the median data interval from −0.5 to 0.5.

### Example 4. Identification of Breast Cancer Subtype by Gene Expression Analysis

The present inventors successfully determined a gene group that exhibited an expression pattern characteristic of squamous cell cancer (hereinafter, referred to as “a group”), a gene group that exhibited an expression pattern characteristic of phyllodes tumor (hereinafter, referred to as “b group”), a gene group that exhibited an expression pattern characteristic of cancer (hereinafter, referred to as “c group”), a gene group that exhibited an expression pattern characteristic of normal tissues (hereinafter, referred to as “d group”), a gene group that exhibited an expression pattern characteristic of normal-like cases (hereinafter, referred to as “e group”), a gene (hereinafter, referred to as “TNBC1”) group that exhibited an expression pattern characteristic of triple negative groups and exhibited an expression pattern characteristic of normal tissues or normal-like cases (hereinafter, referred to as “f group”), a gene (hereinafter, referred to as “TNBC2”) group that exhibited an expression pattern characteristic of triple negative cases (hereinafter, referred to as “g group”), a gene (hereinafter, referred to as “TNBC3”) group that exhibited an expression pattern characteristic of triple negative cases and exhibited an expression pattern also similar to that of a gene defining poorly characterized cancer (indeterminable one) (hereinafter, referred to as “h group”), a gene group that exhibited an expression pattern characteristic of HER2+-like cases (hereinafter, referred to as “i group”), a gene (hereinafter, referred to as HER2 amplification-1″) group that was associated with HER2 amplification and resided chromosomally at a position near the HER2 gene (hereinafter, referred to as “j group”), a gene (hereinafter, referred to as HER2 amplification-2″) group that was associated with HER2 amplification and was other than the j group (hereinafter, referred to as “k group”), a hormone sensitivity-related gene group (hereinafter, referred to as “l group”), ESR1 (hereinafter, referred to as “m group”), a differentiation-related gene group (hereinafter, referred to as “n group”) and a cell cycle-related gene group (hereinafter, referred to as “o group”) from the gene expression profiles obtained in Example 3. 199 genes were determined as genes contained in these a to o groups (identification marker gene sets for identifying a subtype of breast cancer). The following tables show the genes contained in the identification marker gene sets.

TABLE 5A

| Classification | | Symbol | Name | ID |
|---|---|---|---|---|
| a group | Squamous cell cancer | KRTDAP | keratinocyte differentiation-associated protein (KRTDAP), mRNA. | NM_207392 |
| | Squamous cell cancer | LGALS7 | lectin. galactoside-binding, soluble, 7 (galectin 7) (LGALS7), mRNA. | NM_002307 |
| | Squamous cell cancer | PI3 | protease inhibitor 3, skin-derived (SKALP) (PI3). mRNA. | NM_002638 |
| | Squamous cell cancer | SPRR1B | small proline-rich protein 1B (cornifin) (SPRR1B), mRNA. | NM_003125 |
| | Squamous cell cancer | SPRR2A | small proline-rich protein 2A (SPRR2A), mRNA. | NM_005988 |
| | Squamous cell cancer | KRT1 | keratin 1 (epidermolytic hyperkeratosis) (KRT1), mRNA. | NM_006121 |
| | Squamous cell cancer | SERPINB3 | serine (or cysteine) proteinase inhibitor. clade B (ovalbumin), | NM_006919 |
| | | | member 3 (SERPINB3), mRNA. | |
| | Squamous cell cancer | KLK13 | kallikrein 13 (KLK13), mRNA. | NM_015596 |
| b group | Phyllodes tumor | SH3PXD2B | similar to KIAA1295 protein (LOC220776), mRNA. | NM_001017995 |
| | Phyllodes tumor | PTH1R | parathyroid hormone receptor 1 (PTHR1), mRNA. | NM_000316 |
| | Phyllodes tumor | SERPINH1 | serine (or cysteine) proteinase inhibitor, clade H (heat shock protein 47), | NM_001235 |
| | | | member 1, (collagen binding protein 1) (SERPINH1), mRNA. | |
| | Phyllodes tumor | SNAI2 | snail homolog 2 (Drosophila) (SNAI2), mR | NM_003068 |
| | Phyllodes tumor | HAS2 | hyaluronan synthase 2 (HAS2), mRNA. | NM_005328 |
| | Phyllodes tumor | ITLN1 | intelectin 1 (galactofuranose binding) (ITLN1), mRNA. | NM_017625 |
| | Phyllodes tumor | GPR173 | super conserved receptor expressed in br | NM_018969 |
| | Phyllodes tumor | PAGE5 | PAGE-5 protein (PAGE-5), mRNA. | NM_130467 |
| c group | Cancer | FN1 | cellular fibronectin mRNA. | NM_002026 |
| | Cancer | TAP1 | transporter 1, ATP-binding cassette, sub-family B (MDR/TAP) (TAP1), | NM_000593 |
| | | | mRNA. | |
| | Cancer | MMP9 | matrix metalloproteinase 9 (gelatinase B, 92 kDa gelatinase, 92 kDa type | NM_004994 |
| | | | IV collagenase) (MMP9), mRNA. | |
| | Cancer | CTHRC1 | collagen triple helix repeat containing | NM_138455 |

TABLE 5B

| Classification | | Symbol | Name | ID |
|---|---|---|---|---|
| d group | Normal | CD36 | CD36 antigen (collagen type I receptor, | NM_000072 |
| | Normal | LEP | leptin (obesity homolog, mouse) (LEP), mRNA. | NM_000230 |
| | Normal | LIFR | leukemia inhibitory factor receptor (LIFR), mRNA. | NM_002310 |
| | Normal | PLIN1 | perilipin (PLIN), mRNA. | NM_002666 |
| | Normal | TGFBR3 | transforming growth factor, beta receptor III (betaglycan, 300 kDa) (TGFBR3), mRNA. | NM_003243 |
| | Normal | CAVIN2 | serum deprivation response (phosphatidylserine binding protein) (SDPR), mRNA. | NM_004657 |
| | Normal | ADIPOQ | adipocyte, C1Q and collagen domain containing (ACDC), mRNA. | NM_004797 |
| | Normal | GPD1 | glycerol-3-phosphate dehydrogenase 1 (soluble) (GPD1), mRNA. | NM_005276 |
| | Normal | LIPE | lipase, hormone-sensitive (LIPE), mRNA. | NM_005357 |
| | Normal | G0S2 | putative lymphocyte G0/G1 switch gene (G0S2), mRNA. | NM_015714 |
| e group | Normal-like | KIT | v-kit Hardy-Zuckerman 4 feline sarcoma viral oncogene homolog (KIT), mRNA. | NM_000222 |
| | Normal-like | KRT5 | keratin 5 (epidermolysis bullosa simplex, Dowling-Meara/Kobner/Weber-Cockayne | NM_000424 |
| | | | types) (KRT5), mRNA. | |
| | Normal-like | KRT14 | keratin 14 (epidermolysis bullosa simplex, Dowling-Meara, Koebner) | NM_000526 |
| | | | (KRT14), mRNA. | |
| | Normal-like | DST | bullous pemphigoid antigen 1, 230/240 kDa (BPAG1), transcript variant 1e, mRNA. | NM_001723 |
| | Normal-like | KRT15 | keratin 15 (KRT15), mRNA. | NM_002275 |
| | Normal-like | PIGR | polymeric immunoglobulin receptor (PIGR), mRNA. | NM_002644 |
| | Normal-like | WIF1 | WNT inhibitory factor 1 (WIF1), mRNA. | NM_007191 |
| | Normal-like | CAPN6 | calpain 6 (CAPN6), mRNA. | NM_014289 |
| | Normal-like | SYNM | desmuslin (DMN), transcript-variant A, mRNA. | NM_145728 |
| f group | TNBC1 | GABRP | gamma-aminobutyric acid (GABA) A receptor, pi (GABRP), mRNA. | NM_014211 |
| | TNBC1 | ELF5 | E74-like factor 5 (ets domain tranacription factor) (ELF5), transcript variant 2, mRNA. | NM_001422 |
| | TNBC1 | MMP7 | matrix metalloproteinase 7 (matrilysin, uterine). (MMP7), mRNA. | NM_002423 |
| | TNBC1 | SFRP1 | secreted frizzled-related protein 1 (SFRP1), mRNA. | NM_003012 |
| | TNBC1 | MIA | melanoma inhibitory activity (MIA), mRNA. | NM_006533 |
| | TNBC1 | FDCSP | chromosome 4 open reading frame 7 (C4orf7), mRNA. | NM_152997 |

TABLE 5C

| Classification | | Symbol | Name | ID |
|---|---|---|---|---|
| g group | TNBC2 | WIPF3 | cDNA FLJ36931 fia, clone BRACE2005290. | NM_001080529 |
| | TNBC2 | PKP1 | plakophilin 1 (ectodermal dysplasis/skin fragility syndrome) (PKP1), mRNA. | NM_000299 |
| | TNBC2 | ART3 | ADP-ribosyltransferase 3 (ART3), mRNA. | NM_001179 |
| | TNBC2 | EN1 | engrailed homolog 1 (EN1), mRNA. | NM_001426 |
| | TNBC2 | FABP5 | fatty acid binding protein 5 (psoriasis-associated) (FABP5), mRNA. | NM_001444 |
| | TNBC2 | FOXC1 | forkhead box C1 (FOXC1), mRNA. | NM_001453 |
| | TNBC2 | DSG3 | desmoglein 3 (pemphigus vulgaris antigen) (DSG3), mRNA. | NM_001944 |
| | TNBC2 | ETV6 | ets variant gene 6 (TEL oncogene) (ETV6), mRNA. | NM_001987 |
| | TNBC2 | ITGB8 | integrin beta 8 (ITGB8), mRNA. | NM_002214 |
| | TNBC2 | CRABP1 | cellular retinoic acid binding protein 1 (CRABP1), mRNA. | NM_004378 |
| | TNBC2 | DSC2 | desmocollin 2 (DSC2), transcript variant Dac2b, mRNA. | NM_004949 |
| | TNBC2 | KLK7 | kallikrein 7 (chymotryptic, stratum corneum) (KLK7), transcript variant 1, mRNA. | NM_005046 |
| | TNBC2 | MFGE8 | milk fat globule-EGF factor 8 protein (MFGE8), mRNA. | NM_005928 |
| | TNBC2 | MELTF | antigen p97 (melanoma associated) identified by monoclonal antibodies 133.2 and | NM_005929 |
| | | | 96.5 (MFI2), transcipt variant 1, mRNA. | |
| | TNBC2 | PROM1 | prominin 1 (PROM1), mRNA. | NM_006017 |
| | TNBC2 | S100A1 | S100 calcium binding protein A1 (S100A1), mRNA. | NM_006271 |
| | TNBC2 | S100B | S100 calcium binding protein, beta (neural) (S100B), mRNA. | NM_006272 |
| | TNBC2 | MARCO | macrophage receptor with collagenous structure (MARCO), mRNA. | NM_006770 |
| | TNBC2 | SCRG1 | scrapie responsive protein 1 (SCRG1), mRNA. | NM_007281 |
| | TNBC2 | KLK5 | kallikrein 5 (KLK5), mRNA. | NM_012427 |
| | TNBC2 | TM4SF1 | transmembrane 4 superfamily member 1 (TM4SF1), mRNA. | NM_014220 |
| | TNBC2 | RASD2 | RASD family member 2 (RASD2), mRNA. | NM_014310 |
| | TNBC2 | TFCP2L1 | transcription factor CP2-like 1 (TFCP2L1), mRNA. | NM_014553 |
| | TNBC2 | KRT23 | keratin 23 (histone deacetylase inducible) (KRT23), transcript variant 1, mRNA. | NM_015515 |
| | TNBC2 | VGLL1 | vestigial like 1 (Drosophila) (VGLL1), mRNA. | NM_016267 |
| | TNBC2 | TTYH1 | tweety homolog 1 (Drosophila) (TTYH1), mRNA. | NM_020659 |
| | TNBC2 | SPHK1 | sphingosine kinase 1 (SPHK1), mRNA. | NM_021972 |
| | TNBC2 | OGFRL1 | opoid growth factor receptor-like 1 (OGFRL1), mRNA. | NM_024576 |
| | TNBC2 | HORMAD1 | hypothetical protein DKFZp434A1315 (DKFZP434A1315), mRNA. | NM_032132 |
| | TNBC2 | COL27A1 | collagen, type XXVII, alpha 1 (COL27A1), | NM_032888 |
| | TNBC2 | TRIM47 | tripartite motif-containing 47 (TRIM47), mRNA. | NM_033452 |
| | TNBC2 | TSPYL5 | TSPY-like 5 (TSPYL5), mRNA. | NM_033512 |
| | TNBC2 | CYYR1 | cysteine and tyrosine-rich 1 (CYYR1), mRNA. | NM_052954 |
| | TNBC2 | A2ML1 | hypothetical protein FLJ25179 (FLJ25179), mRNA. | NM_144670 |
| | TNBC2 | RFLNA | hypothetical protein LOC144347 (LOC144347), mRNA. | NM_181709 |
| h group | TNBC3 | RNF150 | cDNA FLJ10151 fis, clone HEMBA1003402. | XM_005263150 |
| | TNBC3 | MZB1 | cDNA FLJ32987 fis, clone THYMU1000032. | NM_016459 |
| | TNBC3 | LYZ | lysozyme (renal amyloidosis) (LYZ), mRNA. | NM_000239 |
| | TNBC3 | SPIB | Spi-B transcription factor (Spi-1/PU.1 related) (SPIB), mRNA. | NM_003121 |
| | TNBC3 | KCNK5 | potassium channel, subfamily K, member 5 (KCNK5), mRNA. | NM_003740 |
| | TNBC3 | BCL2A1 | BCL2-related protein A1 (BCL2A1), mRNA. | NM_004049 |
| | TNBC3 | LMO4 | LIM domain only 4 (LMO4), mRNA. | NM_006769 |

TABLE 5D

| Classification | | Symbol | Name | ID |
|---|---|---|---|---|
| i group | HER2+-like | GLYATL2 | BXMAS2-10 (BXMAS2-10), mRNA. | NM_145016 |
| | HER2+-like | GGT1 | gamma-glutamyltransferase 1 (GGT1), transcript variant 1, mRNA. | NM_013421 |
| | HER2+-like | NXPH4 | cDNA FLJ36912 fia, clone BRACE2003847, highly similar to Rattus | NM_007224 |
| | | | norvegicus neurexiphilin 4 (Nph4) mRNA. | |
| | HER2+-like | ATP13A5 | cDNA FLJ16025 fia. clone CTONG2004062, highly similiar to ATPase | NM_198505 |
| | | | subunit 6. | |
| | HER2+-like | PLA2G2A | phospholipase A2, group ILA (platelets, synovial fluid) (PLA2G2A), mRNA. | NM_000300 |
| | HER2+-like | HPGD | hyrdoxyprostaglandin dehydrogenase 15-(NAD) (HPGD), mRNA. | NM_000860 |
| | HER2+-like | FABP7 | fatty acid binding protein 7, brain (FABP7), mRNA. | NM_001446 |
| | HER2+-like | MPP3 | membrane protein; palmitoylated 3 (MAGUK p55 subfamily member 3) | NM_001932 |
| | | | (MPP3), mRNA. | |
| | HER2+-like | HSD17B2 | hydroxysteroid (17-beta) dehydrogenase 2 (HSD17B2), mRNA. | NM_002153 |
| | HER2+-like | S100A9 | S100 calcium binding protein A9 (calgranulin B) (S100A9), mRNA. | NM_002965 |
| | HER2+-like | SLPI | secretory leukocyte protease inhibitor (antileukoproteinase) (SLPI), mRNA. | NM_003064 |
| | HER2+-like | TFAP2B | transcription factor AP-2 beta (activating enhancer binding protein 2 beta) | NM_003221 |
| | | | (TFAP2B), mRNA. | |
| | HER2+-like | THRSP | thyroid hormone responsive (SPOT14 homolog, rat) (THRSP), mRNA. | NM_003251 |
| | HER2+-like | WNT5A | wingless-type MMTV integration site family, member 5A (WNT5A), mRNA. | NM_003392 |
| | HER2+-like | KMO | kynurenine 3-monooxygenase (kynurenine 3-hydroxylase) (KMO), mRNA. | NM_003679 |
| | HER2+-like | KYNU | kynureninase (L-kynurenine hydrolase) (KNYU), mRNA. | NM_003937 |
| | HER2+-like | FASN | fatty acid synthase (FASN), mRNA. | NM_004104 |
| | HER2+-like | LBP | lipopolysaocharide binding protein (LBP), mRNA. | NM_004139 |
| | HER2+-like | PAPSS2 | 3′-phosphoadenosine 5′-phosphosulfate synthase 2 (PAPSS2), mRNA. | NM_004670 |
| | HER2+-like | ENPP3 | ectonucleotide pyrophophatase/phosphodiesterase 3 (ENPP3), mRNA. | NM_005021 |
| | HER2+-like | MPHOSPH6 | M-phase phosphoprotein 6 (MPHOSPH6), mRNA. | NM_005792 |
| | HER2+-like | MPHOSPH6 | M-phase phosphoprotein 6 (MPHOSPH6), mRNA. | NM_005792 |
| | HER2+-like | CLCA2 | chloride channel, calcium activated, family member 2 (CLCA2), mRNA. | NM_006536 |
| | HER2+-like | PXMP4 | peroxisomal membrane protein 4, 24 kDa (PXMP4), transcript variant 1, mRNA. | NM_007238 |
| | HER2+-like | SRPK3 | serine/threonine kinase 23 (STK23), mRNA. | NM_014370 |
| | HER2+-like | SERHL2 | kraken-like (dJ222E13.1), mRNA. | NM_014509 |
| | HER2+-like | VPS13D | vacuolar protein sorting 13D (yeast) (VP | NM_015376 |
| | HER2+-like | ABCA12 | ATP-binding cassette, sub-family A (ABC1), member 12 (ABCA12), | NM_015657 |
| | | | transcript variant 2, mRNA. | |
| | HER2+-like | TRPV6 | transient receptor potential cation chan | NM_018646 |
| | HER2+-like | SRD5A3 | hypothetical protein FLJ13352 (FLJ13352), mRNA. | NM_024592 |
| | HER2+-like | MAB21L4 | hypothetical protein FLJ22671 (FLJ22671), mRNA. | NM_024861 |
| | HER2+-like | C21orf58 | chromosome 21 open reading frame 58 (C21orf58), transcript variant 1, mRNA. | NM_058180 |
| | HER2+-like | TMEM45B | hypothetical protein BC016153 (LOC120224), mRNA. | NM_138788 |
| | HER2+-like | NUDT8 | nudix (nucleoside diphosphate linked moiety X)-type motif 8 (NUDTS), | NM_181843 |
| | | | mRNA. | |
| | HER2+-like | CLDN8 | claudin 8 (CLDN8), mRNA. | NM_199328 |
| | HER2+-like | KRT7 | keratin 7 (KRT7), mRNA. | NM_005556 |
| | HER2+-like | TMEM86A | hypothetical protein FLJ90119 (FLJ90119), mRNA. | NM_153347 |
| | HER2+-like | MBOAT1 | cDNA FLJ16207 fia, clone CTONG2019822 | NM_001080480 |

TABLE 5E

| Classification | | Symbol | Name | ID |
|---|---|---|---|---|
| j group | HER2 amplification-1 | PGAP3 | perl-like domain containing 1 (PERLD1), mRNA. | NM_033419 |
| | HER2 amplification-1 | STARD3 | START domain containing 3 (STARD3), mRNA. | NM_006804 |
| | HER2 amplification-1 | ERBB2 | v-erb-b2 erythroblastic leukemia viral oncogene homolog 2. neuro/ | NM_004448 |
| | | | glioblastoma derived oncogene homolog (avian) (ERBB2), mRNA. | |
| | HER2 amplification-1 | MIEN1 | chromosome 17 open reading frame 37 (C17orf37), mRNA. | NM_032339 |
| | HER2 amplification-1 | GRB7 | growth factor receptor-bound protein 7 (GRB7), mRNA. | NM_005310 |
| k group | HER2 amplification-2 | GSDMB | gasdermin-like (GSDML), mRNA. | NM_018530 |
| | HER2 amplification-2 | ORMDL3 | ORM1-like 3 (S. cerevisiae) (ORMDL3), mRNA. | NM_139280 |
| | HER2 amplification-2 | MED24 | thyroid hormone receptor associated protein 4 (THRAP4), mRNA. | NM_014815 |
| | HER2 amplification-2 | MSL1 | cDNA FLJ30816 fis. clone FEBRA2001571. | NM_001012241 |
| | HER2 amplification-2 | CASC3 | cancer susceptibility candidate 3 (CASC3), mRNA. | NM_007359 |
| | HER2 amplification-2 | WIPF2 | WIRE protein (WIRE), mRNA. | NM_133264 |

TABLE 5F

| 1 group | Classification | Symbol | Name | ID |
|---|---|---|---|---|
| | Hormone | GFRA1 | GDNF family receptor alpha 1 | NM_005264 |
| | sensitivity | | (GFRA1), transcript variant 1, mRNA. | |
| | Hormone | MAPT | microtubule-associated protein tau | NM_016835 |
| | sensitivity | | (MAPT), transcript variant 1, mRNA. | |
| | Hormone | EVL | Enah/Vasp-like (EVL), mRNA. | NM_016337 |
| | sensitivity | | | |
| | Hormone | CA12 | carbonic anhydrase XII (CA12), | NM_206925 |
| | sensitivity | | transcrip | |
| | Hormone | LONRF2 | cDNA FLJ31811 fis, clone | NM_198461 |
| | sensitivity | | NT2RI2009402. | |
| | Hormone | CYP2B6 | cytochrome P450-IIB (hIIB3) mRNA, | NM_000767 |
| | sensitivity | | complete cds. | |
| | Hormone | PARD6B | par-6 partitioning defective 6 homolog b | NM_032521 |
| | sensitivity | | | |
| | Hormone | TBC1D9 | KIAA0882 protein (KIAA0882), mRNA. | NM_015130 |
| | sensitivity | | | |
| | Hormone | ESR1 | estrogen receptor 1 (ESR1), mRNA. | NM_000125 |
| | sensitivity | | | |
| | Hormone | NAT1 | N-acetyltransferase 1 (arylamine N- | NM_000662 |
| | sensitivity | | acetyltransferase) (NAT1), mRNA. | |
| | Hormone | CHAD | chondroadherin (CHAD), mRNA. | NM_001267 |
| | sensitivity | | | |
| | Hormone | HPN | hepsin (transmembrane protease, serine 1) | NM_002151 |
| | sensitivity | | (HPN), transcript variant 2, mRNA | |
| | Hormone | IL6ST | interleukin 6 signal transducer (gp130, | NM_002184 |
| | sensitivity | | oncostatin M receptor) (IL6ST), transcript | |
| | | | variant 1, mRNA. | |
| | Hormone | STC2 | stanniocalcin 2 (STC2), mRNA. | NM_003714 |
| | sensitivity | | | |
| | Hormone | SLC39A6 | solute carrier family 39 (zinc transporter), | NM_012319 |
| | sensitivity | | member 6 (SLC39A6), mRNA. | |
| | Hormone | GREB1 | GREB1 protein (GREB1), transcript | NM_014668 |
| | sensitivity | | variant a, mRNA. | |
| | Hormone | GASK1B | hypothetical protein DKFZp434L142 | NM_016613 |
| | sensitivity | | (DKFZp434L142), mRNA. | |
| | Hormone | DBNDD2 | chromosome 20 open reading frame 35 | NM_018478 |
| | sensitivity | | (C20orf35), mRNA. | |
| | Hormone | ENPP5 | ectonucleotide pyrophosphatase/phospho- | NM_021572 |
| | sensitivity | | diesterase 5 (putative function) (ENPP5), | |
| | | | mRNA. | |
| | Hormone | THSD4 | hypothetical protein FLJ13710 (FLJ13710), | NM_024817 |
| | sensitivity | | mRNA. | |
| | Hormone | ZNF703 | hypothetical protein FLJ14299 (FLJ14299), | NM_025069 |
| | sensitivity | | mRNA. | |
| | Hormone | TCEAL3 | hypothetical protein MGC15737 (MGC15737), | NM_032926 |
| | sensitivity | | mRNA. | |
| | Hormone | FGD3 | FGD1 family, member 3 (FGD3), mRNA. | NM_033086 |
| | sensitivity | | | |
| | Hormone | KCNE4 | potassium voltage-gated channel, Isk-related | NM_080671 |
| | sensitivity | | family, member 4 (KCNE4), mRNA. | |
| | Hormone | KCNE4 | potassium voltage-gated channel, Isk-related | NM_080671 |
| | sensitivity | | family, member 4 (KCNE4), mRNA. | |
| | Hormone | ARMT1 | chromosome 6 open reading frame 211 | NM_024573 |
| | sensitivity | | (C6orf211), mRNA. | |
| | Hormone | MAGED2 | melanoma antigen, family D, 2 (MAGED2), | NM_177433 |
| | sensitivity | | transcript variant 2, mRNA. | |
| | Hormone | CELSR1 | cadherin, EGF LAG seven-pass G-type receptor 1 | NM_014246 |
| | sensitivity | | (flamingo homolog, Drosphila) (CELSR1), mRNA. | |
| | Hormone | INPP5J | phosphatidylinositol (4,5) biphosphate 5- | NM_001002837 |
| | sensitivity | | phosphatase, A (PIB5PA), mRNA. | |
| | Hormone | PADI2 | peptidyl arginine deiminase, type II (PADI2), | NM_007365 |
| | sensitivity | | mRNA. | |
| | Hormone | PPP1R1B | protein phosphatase 1, regulatory (inhibitor) subunit | NM_032192 |
| | sensitivity | | 1B (dopamine and cAMP regulated phosphoprotein, | |
| | | | DARPP-32) (PPP1R1B), mRNA. | |

TBALE 5G

| Classification | | Symbol | Name | ID |
|---|---|---|---|---|
| n group | Differentiation | GATA3 | GATA binding protein 3 (GATA3), mRNA. | NM_002051 |
| | Differentiation | SCGB2A2 | secretoglobin, family 2A, member 2 (SCGB2A2), mRNA. | NM_002411 |
| | Differentiation | TFF3 | trefoil factor 3 (intestinal) (TFF3), mRNA. | NM_003226 |
| | Differentiation | FOXA1 | forkhead box A1 (FOXA1), mRNA. | NM_004496 |
| | Differentiation | XBP1 | X-box binding protein 1 (XBP1), mRNA. | NM_005080 |
| | Differentiation | AGR2 | anterior gradient 2 homolog (Xenopus laevia) (AGR2), mRNA. | NM_006408 |
| | Differentiation | KIAA0040 | KIAA0040 gene product (KIAA0040), mRNA. | NM_014656 |
| | Differentiation | MLPH | melanophilin (MLPH), mRNA. | NM_024101 |
| | Differentiation | MUCL1 | small breast epithelial mucin (LOC118430), mRNA. | NM_058173 |
| | Differentiation | TMC4 | tranamembrane channel-like 4 (TMC4), mRNA. | NM_144686 |
| | Differentiation | ZG16B | similar to common salivary protein 1 (LOC124220), mRNA. | NM_145252 |
| o group | Cell cycle | RRM2 | ribonucleotide reductase M2 polypeptide (RRM2), mRNA. | NM_001034 |
| | Cell cycle | CCNA2 | cyclin A2 (CCNA2), mRNA. | NM_001237 |
| | Cell cycle | CDC20 | CDC20 cell division cycle 20 homolog (S. cerevisiae) (CDC20), mRNA. | NM_001255 |
| | Cell cycle | CDK1 | cell division cycle 2, G1 to S and G2 to M (CDC2), transcript variant 1, mRNA. | NM_001786 |
| | Cell cycle | CKS2 | CDC28 protein kinase regulatory subunit 2 (CKS2), mRNA. | NM_001827 |
| | Cell cycle | H2AFX | H2A histone family, member X (H2AFX), mRNA. | NM_002105 |
| | Cell cycle | H2AFZ | H2A histone family, member Z (H2AFZ), mRNA. | NM_002106 |
| | Cell cycle | KPNA2 | karyopherin alpha 2 (RAG cohort 1, importin alpha 1) (KPNA2), mRNA. | NM_002266 |
| | Cell cycle | MKI67 | antigen identified by monoclonal antibody Ki-67 (MKI67), mRNA. | NM_002417 |
| | Cell cycle | MYBL2 | v-myb myeloblastosis viral oncogene homolog (avian)-like 2 (MYBL2), mRNA. | NM_002466 |
| | Cell cycle | GGH | gamma-glutamyl hydrolase (conjugase, folylpolygammaglutamyl hydrolase) | NM_003878 |
| | | | (GGH), mRNA. | |
| | Cell cycle | PTTG1 | pituitary tumor-transforming 1 (PTTG1), mRNA. | NM_004219 |
| | Cell cycle | DDX11 | DEAD/H (Asp-Glu-Ala-Asp/His) box polypeptide 11 (CHL1-like helicase | NM_004399 |
| | | | homolog, S. cerevisiae) (DDX11), transcript variant 2, mRNA. | |
| | Cell cycle | CCNB2 | cyclin B2 (CCNB2), mRNA. | NM_004701 |
| | Cell cycle | UBE2C | ubiquitin-conjugating enzyme E2C (UBE2C), transcript variant 1, mRNA. | NM_007019 |
| | Cell cycle | ATAD2 | ATPase family, AAA domain containing 2 (ATAD2), mRNA. | NM_014109 |
| | Cell cycle | UBE2T | HSPC150 protein similar to ubiquitin-conjugating enzyme (HSPC150), mRNA. | NM_014176 |
| | Cell cycle | JPT1 | hematological and neurological expressed 1 (HN1), mRNA. | NM_016185 |
| | Cell cycle | CKAP2 | cytoskeleton associated protein 2 (CKAP2), mRNA. | NM_018204 |
| | Cell cycle | ANLN | anillin, actin binding protein (scraps homolog, Drosophila) (ANLN), mRNA. | NM_018685 |
| | Cell cycle | FOXM1 | forkhead box M1 (FOXM1), transcript variant 2, mRNA. | NM_021953 |
| | Cell cycle | CDCA3 | cell division cycle associated 3 (CDCA3), mRNA. | NM_031299 |
| | Cell cycle | MCM4 | MCM4 minichromosome maintenance deficient 4 (S. cerevisiae) (MCM4), | NM_182746 |
| | | | transcript variant 2, mRNA. | |

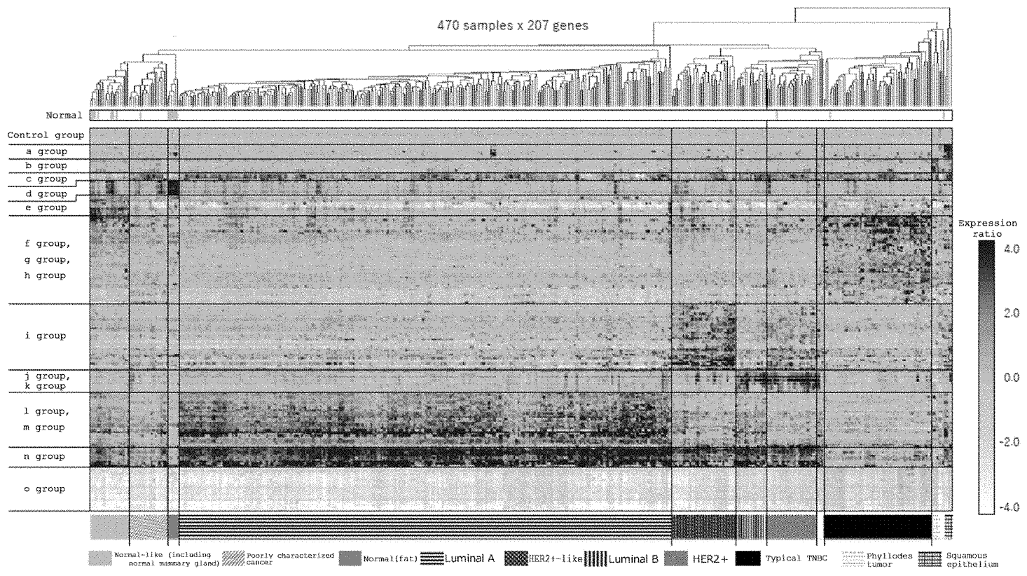

The gene expression level of each gene was measured (data not shown) as to 207 genes in total involving the eight internal standard genes and the identification marker gene sets consisting of the 199 genes, followed by cluster analysis. The cluster analysis was conducted by a gene averaging technique based on a Euclidean distance using ExpressionView Pro software (MicroDiagnostic). The results of the cluster analysis are shown in FIG. 4. As shown in FIG. 4, as a result of conducting hierarchical cluster analysis on the basis of the expression profiles of the 207 extracted genes, the genes contained in the a to o groups exhibited an expression ratio characteristic of each subtype, whereas no variation in expression was observed in the genes of a control group for any subtype. By the hierarchical cluster analysis, the subtypes of breast cancer were able to be classified into clusters of a normal-like group, an indeterminable group, a normal group, a luminal A group, a HER2+-like group, a luminal B group, a HER2+ group, a triple negative group, and other groups.

The following tables show sequence information on the probes against the 207 genes used in Examples 3 and 4.

TABLE 6A

| Symbol | Sequence ID | Sequence of probe | SEQ ID NO |
|---|---|---|---|
| FBXW5 | NM_018998 | ACCACTGGCTGCCTCACCTACTCCCCACACCAGATCGGCATCAAGCAGATCCTGCCACACCAGATGACCACGGCAGGGCC | SEQ ID NO: 9 |
| PITPNM1 | NM_004910 | CACTCCAGCCTCTTTCTGGAGGAGCTGGAGATGCTGGTGCCCTCAACACCCACCTCTACTAGCGGTGCCTTCTGGAAGGG | SEQ ID NO: 10 |
| MLLT1 | NM_005934 | ATCTGATCGAGGAGACTGGCCACTTCAATGTCACCAACACCACCTTCGACTTCGACCTCTTCTCCCTGGACGAGACCACC | SEQ ID NO: 11 |
| WDR1 | NM_017491 | AGCCTGGCCTGGCTGGACGAGCACACGCTGGTCACGACCTCCCATGATGCCTCTGTCAAGGAGTGGACAATCACCTACTG | SEQ ID NO: 12 |
| ABCF3 | NM_018358 | TGACTATGCCCTGCCCCAACTTCTACATTCTGGATGAACCCACAAACCACCTGGACATGGAGACCATTGAGGCTCTGGGC | SEQ ID NO: 13 |
| NDUFS7 | NM_022407 | ACTATTCCTACTCGGTGGTGAGGGGCTGCGACCGCATCGTGCCCGTGGACATCTACATCCCAGGCTGCCCACCTACGGCC | SEQ ID NO: 14 |
| FAM234A | NM_032039 | TGGCACCGACAGACAGATCCTGTTTCTGGACCTTGGCACTGGAGCCGTCCTGTGTAGCCTAGCCCTCCCGAGCCTCCCTG | SEQ ID NO: 15 |
| AP2A1 | NM_130787 | AGCATTCCAACGCCAAGAACGCCATCCTCTTCGAGACCATCAGCCTCATCATCCACTATGACAGTGAGCCCAACCTCCTG | SEQ ID NO: 16 |
| KRTDAP | NM_207392 | CTTTGAGTCTATCAAAAGGAAACTTCCTTTCCTCAACTGGGATGCCTTTCCTAAGCTGAAAGGACTGAGGAGCGCAACTC | SEQ ID NO: 17 |
| SERPINB3 | NM_006919 | TGGAAGAGAGCTATGACCTCAAGGACACGTTGAGAACCATGGGAATGGTGGATATCTTCAATGGGGATGCAGACCTCTCA | SEQ ID NO: 18 |
| SPRR2A | NM_ [symbol] | CCTCAGCAGTGCCAGCAGAAATATCCTCCTGTGACACCTTCCCCACCCTGCCAGTCAAAGTATCCACCCAAGAGCAAGTA | SEQ ID NO: 19 |
| SPKK1B | NM_003125 | GTTTTCAGCTGCTCAGAATTCATCTGAAGAGAGACTTAAGATGAAAGCAAATGATTCAGCTCCCTTATACCCCCATTAAA | SEQ ID NO: 20 |
| KLK13 | NM_015596 | CCGTGTCTCAAGATACGTCCTGTGGATCCGTGAAACAATCCGAAAATATGAAACCCAGCAGCAAAAATGGTTGAAGGGCC | SEQ ID NO: 21 |
| KRT1 | NM_006121 | TCCGAAGAAGAGTGGACCAACTGAAGAGTGATCAATCTCGGTTGGATTCGGAACTGAAGAACATGCAGGACATGGTGGAG | SEQ ID NO: 22 |
| LGALS7 | NM_002307 | GCAGCCCTTCGAGGTGCTCATCATCGCGTCAGACGACGGCTTCAAGGCCGTGGTTGGGGACGCCCAGTACCACCACTTCC | SEQ ID NO: 23 |
| PI3 | NM_002638 | GTTCCTGTTAAAGGTCAAGACACTGTCAAAGGCCGTGTTCCATTCAATGGACAAGATCCCGTTAAAGGACAAGTTTCAGT | SEQ ID NO: 24 |
| SERPINH1 | NM_001235 | CCCAGGCTGTTCTACGCCGACCACCCCTTCATCTTCCTAGTGCGGGACACCCAAAGCGGCTCCCTGCTATTCATTGGGCG | SEQ ID NO: 25 |
| SNAI2 | NM_008068 | GCTCCTTCCTGGTCAAGAAGCATTTCAACGCCTCCAAAAAGCCAAACTACAGCGAACTGGACACACATACAGTGATTATT | SEQ ID NO: 26 |
| GPR173 | NM_018969 | CACGGCTCTTCATGGACCTTCAGTGCACTCAGCTGCAAGATTGTGGCCTTTATGGCCGTGCTCTTTTGCTTCCATGCGGC | SEQ ID NO: 27 |
| HAS2 | NM_005328 | CAGACAGTTCTAATTGTTGGAACGTTGCTCTATGCATGCTATTGGGTCATGCTTTTGACGCTGTATGTAGTTCTCATCAA | SEQ ID NO: 28 |
| PTH1R | NM_000316 | TTTTGTCGCAATCATATACTGTTTCTGCAATGGCGAGGTACAAGCTGAGATCAAGAAATCTTGGAGCCGCTGGACACTGG | SEQ ID NO: 29 |
| PAGE5 | NM_ [symbol] | TGATGTGGAAGCTTTTCAACAGGAACTGGCTCTGCTTAAGATAGAGGATGCACCTGGAGATGGTCCTGATGTCAGGGAGG | SEQ ID NO: 30 |
| ITLN1 | NM_017625 | TGCATTTGATGGCCTGTATTTTCTCCGCACTGAGAATGGTGTTATCTACCAGACCTTCTGTGACATGACCTCTGGGGGTG | SEQ ID NO: 31 |
| SH3PKD2B | NM_001017995 | AGATGCCACTCCCCAGAATCCCTTCTTGAAGTCCAGACCTCAGGTTAGGCCAAAACCAGCTCCTTCCCCCAAAACGGAGC | SEQ ID NO: 32 |
| TAP1 | NM_000593 | ACCCAGTGGTCTGTTGACTCCCTTACACTTGGAGGGCCTTGTCCAGTTCCAAGATGTCTCCTTTGCCTACCCAAACCGCC | SEQ ID NO: 33 |
| FN1 | NM_002026 | AAGACATACCACGTAGGAGAACAGTGGCAGAAGGAATATCTCGGTGCCATTTGCTCCTGCACATGCTTTGGAGGCCAGCG | SEQ ID NO: 34 |
| CTHRC1 | NM_138455 | GAAAGCTTTGAGGAGTCCTGGACACCCAACTACAAGCAGTGTTCATGGAGTTCATTGAATTATGGCATAGATCTTGGGAA | SEQ ID NO: 35 |
| MMP9 | NM_004994 | GTGAGTTCCCGGAGTGAGTTGAACCAGGTGGACCAAGTGGGCTACGTGACCTATGACATCCTGCAGTGCCCTGAGGACTA | SEQ ID NO: 36 |

[symbol]

indicates data missing or illegible when filed

TABLE 6B

| Symbol | Sequence ID | Sequence of probe | SEQ ID NO |
|---|---|---|---|
| CRABP1 | NM_004378 | GAGGAGGAGACCGTGGACGGACGCAAGTGCAGGAGTTTAGCCACTTGGGAGAATGAGAACAAGATCCACTGCACCCAAAC | SEQ ID NO: 62 |
| PROM1 | NM_006017 | CGCACAGGGAATGGATTGTTGGAGAGAGTAACTAGGATTCTAGCTTCTCTGGATTTTGCTCAGAACTTCATCACAAACAA | SEQ ID NO: 63 |
| KRT23 | NM_015515 | AAGATCAAGGCCATAACCCAGGAGACCATCAACGGAAGATTAGTTCTTTGTCAAGTGAATGAAATCCAAAAGCACGCATG | SEQ ID NO: 64 |
| S100A1 | NM_006271 | ATGCCCAGAAGGATGTGGATGCTGTGGACAAGGTGATGAAGGAGCTAGACGAGAATGGAGACGGGGAGGTGGACTTTCCAG | SEQ ID NO: 65 |
| WIPF3 | NM_001080529 | TGCGAAATGGAAGCCTGCACATCATTGATGACTTCGAGTCTAAATTCACGTTCCATTCTGTGGAAGACTTTCCCCCTCCG | SEQ ID NO: 66 |
| CRRR1 | NM_052954 | CTGCTCTTTGTCTACGCAGATGATTGCCTTGCTCAGGTGTGGCAAAGATTGCAAATCTTACTGCTGTGATGGAACCACGCC | SEQ ID NO: 67 |
| TFCP2L1 | NM_014553 | TGTACCACGCCATCTTCCTGGAAGAGCTGACCACCTTGGAGCTGATTGAGAAGATTGCCAACTGTACAGCATCTCCCCC | SEQ ID NO: 68 |
| DSC2 | NM_004949 | GTTGGGCATAGCATTGCTCTTTTGCATCCTGTTTACGCTGGTCTGTGGGGCTTCTGGGACGTCTAAACAACCAAAAGTAA | SEQ ID NO: 69 |
| MFGE8 | NM_005928 | TGGCAGCAGTAAGATCTTCCCTGGCAACTGGGACAACCACTCCCACAAGAAGAACTTGTTTGAGACGCCCATCCTGGCTC | SEQ ID NO: 70 |
| KLK7 | NM_005046 | CAACCCAATGACCCAGGAGTCTACACTCAAGTGTGCAAGTTCACCAAGTGGATAAATGACACCATGAAAAAGCATCGCTA | SEQ ID NO: 71 |
| KLK5 | NM_012427 | CTTCTGGGGGTCACAGAGCATGTTCTCGCCAACAATGATGTTTCCTGTGACCACCCCTCTAACACCGTGCCCTCTGGGAG | SEQ ID NO: 72 |
| DSG3 | NM_001944 | CCCATCCCATAGAAGTCCAGCAGACAGGATTTGTTAAGTGCCAGACTTTGTCAGGAAGTCAAGGAGCTTCTGCTTTGTCCG | SEQ ID NO: 73 |
| TTYH1 | NM_020659 | GACTACGATGACACAGACGATGACGACCCTTTCAACCCTCAGGAATCCAAGCGCTTTGTGCAGTGGCAGTCGTCTATCTG | SEQ ID NO: 74 |
| SCRG1 | NM_007281 | TTCAGCGAATTGCTCTGCTGCCCAAAAGACGTTTTCTTTGGACCAAAGATCTCTTTCGTGATTCCTTGCAACAATCAATG | SEQ ID NO: 75 |
| S100B | NM_006272 | GGGAGACAAGCACAAGCTGAAGAAATCCGAACTCAAGGAGCTCATCAACAATGAGCTTTCCCATTTCTTAGAGGAAATCA | SEQ ID NO: 76 |
| ETV6 | NM_001987 | CTCATTCAGGTGATGTGCTCTATGAACTCCTTCAGCATATTCTGAAGCAGAGGAAACCTCGGATTCTTTTTTCACCATTC | SEQ ID NO: 77 |
| OGFRL1 | NM_ [symbol] | AAGAAATACAGAGAAGGACAGTAATGCTGAGAACATGAATTCTCAACCTGAGAAAACAGTTACTACTCCCACAGAAAAAA | SEQ ID NO: 78 |
| MELTF | NM_005929 | ACAATAAGAACGGGTTCAAAATGTTCGACTCCTCCAACTATCATGGCCAAGACCTGCTTTTCAAGGATGCCACCGTCCGG | SEQ ID NO: 79 |
| HORMAD1 | NM_082132 | AGTCCTCCATCACTTTGATTCTTCTAGTCAAGAGTCAGTGCCAAAAAGGAGAAAGTTTAGTGAACCAAAGGAACATATAT | SEQ ID NO: 80 |
| PKP1 | NM_000299 | ATGTGGTCCAGCAAGGAACTGCAGGGTGTCCTCAGACAGCAAGGTTTCGATAGGAACATGCTGGGAACCTTAGCTGGGGC | SEQ ID NO: 81 |
| FOXC1 | NM_001453 | GAACAACTCTCCAGTGAACGGGAATAGTAGCTGTCAAATGGCCTTCCCTTCCAGCCAGTCTCTGTACCGCACGTCCGGAG | SEQ ID NO: 82 |
| ITGB8 | NM_002214 | TCATGTGCTCTCATGGAACAACAGCATTATGTCGACCAAACTTCAGAATGTTTCTCCAGCCCAAGCTACTTGAGAATATT | SEQ ID NO: 83 |
| VGLL1 | NM_016267 | AGTACCAGCCTTCCAAATGAAACTCTTTCAGAGTTAGAGACACCTGGGAAATACTCACTTACACCACCAAACCACTGGGG | SEQ ID NO: 84 |
| ART3 | NM_001179 | GCTTGAAGACCATGGTGAGAAAAACCAGAAGCTTGAAGACCATGGTGTGAAAATCCTTGAACCCACCCAAATACCTGCTC | SEQ ID NO: 85 |
| EN1 | NM_001426 | TCTCTATTCCCAGTATAAGGGACGAAACTGCGAACTCCTTAAAGCTCTATCTAGCCAAACCGCTTACGACCTTGTATATA | SEQ ID NO: 86 |
| SPHK1 | NM_021972 | TTCCGCTTGGAGCCCAAGGATGGGAAAGGTGTGTTTGCAGTGGATGGGGAATTGATGGTTAGCGAGGCCGTGCAGGGCCA | SEQ ID NO: 87 |
| [symbol] | NM_033452 | GCAGACAAGTTCCTGCAGCTGTTTGGAACCAAAGGTGTCAAGAGGGTGCTGTGTCCTATCAACTACCCCTTGTCGCCCAC | SEQ ID NO: 88 |
| COL27A1 | NM_032888 | CTTGGCTGCTCCTCTGACACCATCGAGGTCTCCTGCAACTTCACTCATGGTGGACAGACGTGTCTCAAGCCCATCACGGC | SEQ ID NO: 89 |
| RFLNA | NM_181709 | CCCACGCATGAGATCCGCTGCAACTCTGAGGTCAAGTACGCCTCGGAGAAGCATTTCCAGGACAAGGTCTTCTATGCGCC | SEQ ID NO: 90 |
| RASD2 | NM_014310 | GTCCTTCGATGAGGTCAAGCGCCTTCAGAAGCAGATCCTGGAGGTCAAGTCCTGCCTGAAGAACAAGACCAAGGAGGCGG | SEQ ID NO: 91 |
| A2ML1 | NM_144670 | AAACCAGCAACCATCAAGGTCTATGACTACTACCTACCAGATGAACAGGCAACAATTCAGTATTCTGATCCCTGTGAATG | SEQ ID NO: 92 |
| MARCO | NM_006770 | GGACAATTTGCGATGACGAGTGGCAAAATTCTGATGCCATTGTCTTCTGCCGCATGCTGGGTTACTCCAAAGGAAGGGCC | SEQ ID NO: 93 |
| TSPYL5 | NM_033512 | TCAACGAAGAATTGTGGCCCAATCCCTTGCAGTTCTACCTTTTGAGTGAAGGGGCTCGTGTAGAGAAAGGAAAGGAAAAA | SEQ ID NO: 94 |
| TM4SF1 | NM_014200 | GATGCTTTCTTCTGTATTGGCTGCTCTCATTGGAATTGCAGGATCTGGCTACTGTGTCATTGTGGCAGCCCTTGGCTTAG | SEQ ID NO: 95 |
| FABP5 | NM_001444 | ACAATAACAAGAAAATTGAAAGATGGGAAATTAGTGGTGGAGTGTGTCATGAACAATGTCACCTGTACTCGGATCTATGA | SEQ ID NO: 96 |
| SPIB | NM_003121 | CTTCAGCTGTCTGTACCCAGATGGCGTCTTCTATGACCTGGACAGCTGCAAGCATTCCAGCTACCCTGATTCAGAGGGGG | SEQ ID NO: 97 |
| BCL2A1 | NM_004049 | TGCCAGAACACTATTCAACCAAGTGATGGAAAAGGAGTTTGAAGACGGCATCATTAACTGGGGAAGAATTGTAACCATAT | SEQ ID NO: 98 |
| MZB1 | NM_016459 | CTCCCGGAACTGGCAGGACTACGGAGTTCGAGAAGTGGACCAAGTGAAACGTCTCACAGGCCCAGGACTTAGCGAGGGGC | SEQ ID NO: 99 |
| KCNK5 | NM_003740 | AAGGACGTCAACATCTTCAGCTTTCTTTCCAAGAAGGAAGAGACCTACAACGACCTCATCAAGCAGATCGGGAAGAAGGC | SEQ ID NO: 100 |
| LMO4 | NM_006769 | ACATGATAGACCTACAGCTCTCATCAATGGCCATTTGAATTCACTTCAGAGCAATCCACTACTGCCAGACCAGAAGGTCT | SEQ ID NO: 101 |
| RNF150 | NM_005263150 | TCTAAGTTTCTTTTCCTTTTCTGTCTGTATCTGTTTTTCTCTGACTGCCTATATCTTACTTTGTATACCCATACATAAAT | SEQ ID NO: 102 |
| LYZ | NM_000239 | GTCATTTATCCTGCAGTGCTTTGCTGCAAGATAACATCGCTGATGCTGTAGCTTGTGCAAAGAGGGTTGTCCGTGATCCA | SEQ ID NO: 103 |

[symbol]

indicates data missing or illegible when filed

TABLE 6C

| Symbol | Sequence ID | Sequence of probe | SEQ ID NO |
|---|---|---|---|
| [symbol] | NM_ [symbol] | CATGGTGGAGCTGCTGCTGCTGCAGAACGCACAGGTGCACCAGTTGGTCCTGCAGAACTGGATGCTCAAGGCCCTGCCC | SEQ ID NO: 104 |
| [symbol] | NM_198505 | CTTCTCTATGTGAAGCAGCAGCCTTGGTATTGTGAGGTCTACCAATACAGTGAGTGTTTTCTGGCCAACCAAAGCCCATA | SEQ ID NO: 105 |
| [symbol] | NM_151843 | ACCGTGGTGCCAGTGCTTGCTGGTGTAGGCCCACTGGATCCCCAGAGCCTCAGGCCAACTCGGAGGAGGTGAGCTGGGG | SEQ ID NO: 106 |
| [symbol] | NM_002153 | GACTGACTACAAACAATGCATGGCCGTGAACTTCTTTGGAACTGTGGAGGTCACAAAGACGTTTTTGCCTCTTCTTAGAA | SEQ ID NO: 107 |
| ABCA12 | NM_015657 | AAGTCCTATGAAACTGCTGATACCAGCAGCCAAGGTTCCACTATAAGTGTTGACTCACAAGATGACCAGATGGAGTCTTA | SEQ ID NO: 108 |
| ENPP5 | NM_005021 | AACACTGATGTTCCCATCCCAACACACTACTTTGTGGTGCTGACCAGTTGTAAAAACAAGAGCCACACACCGGAAAACTG | SEQ ID NO: 109 |
| WNT5A | NM_003392 | GCCACTGCAAGTTCCACTGGTGCTGCTACGTCAAGTGCAAGAAGTGACGGAGATCGTGGACCAGTTTGTGTGCAAGTAG | SEQ ID NO: 110 |
| MPPS | NM_001932 | GGTGCCTACAGCCAGCTCAAAGTGGTCTTAGAGAAGCTGAGCAAGGACACTCACTGGGTACCTGTTAGTTGGGTCAGGTA | SEQ ID NO: 111 |
| [symbol] | NM_015378 | AATCGTGGATGGCAGATTACTGTAAAGATGACAAGGACATAGAGTCAGCTAAATCAGAAGACTGGATGGGCTCTTCGGTG | SEQ ID NO: 112 |
| [symbol] | NM_007238 | ACCTACCTCTATGAGGACAGCAATGTATGGCACGACATCTCAGACTTCCTCGTCTATAACAAGAGCCGTCCCTCCAATTA | SEQ ID NO: 113 |
| FFT1 | NM_013421 | CCGGTCAGCGGGATCCTGTTCAATAATGAAATGGACGACTTCAGCTCTCCCAGCATCACCAACGAGTTTGGGGTACCCCC | SEQ ID NO: 114 |
| [symbol] | NM_015646 | AGCATGAACGCCAAGGAATGTACGTTGAGAATCACTGCTCCAGGCCTGCATTACTCCTTCAGCTCTGGGGCAGAGGAAGC | SEQ ID NO: 115 |
| [symbol] | NM_024861 | AACATCCAGGATAAGGACCGGATCTCTGCCATGCAGAGCATCTTCCAGAAGACCAGGACTCTGGGAGGCGAGGAGAGCTG | SEQ ID NO: 116 |
| CLDN8 | NM_199328 | CCTTCCCATCGCACAACCCAAAAAAGTTATCACACCGGAAAGAAGTCACCGAGCGTCTACTCCAGAAGTCAGTATGTGTA | SEQ ID NO: 117 |
| SBP | NM_ [symbol] | AACTATTACATCCTTAACACCCTCTACCCCAAGTTCAATGATAAGTTGGCCGAAGGCTTCCCCCTTCCTCTGCTGAAGCG | SEQ ID NO: 118 |
| [symbol] | NM_024592 | GGAGACTGGTTTGAATATGTTTCTTCCCCTAACTACTTAGCAGAGCTGATGATCTACGTTTCCATGGCCGTCACCTTTGG | SEQ ID NO: 119 |
| [symbol] | NM_004670 | ATTCCGAGTGGCTGCCTACAACAAAGCCAAAAAAGCCATGGACTTCTATGATCCAGCAAGGCACAATGAGTTTGACTTCA | SEQ ID NO: 120 |
| [symbol] | NM_ [symbol] | ACTATTCTCTTGTTTACTGCCTTTTGACTCGGATGAAGAGACACGGAAGGGGAGAAATCATTGGAATTCAGAAGCTGAAT | SEQ ID NO: 121 |
| [symbol] | NM_ [symbol] | AAAGCTTATGGCTCTGTGATGATATTAGTGACCAGCGGAGATGATAAGCTTCTTGGCAATTGCTTACCCACTGTGCTCAG | SEQ ID NO: 122 |
| FASN | NM_004104 | TGGTCTTGAGAGATGGCTTGCTGGAGAACCAGACCCCAGAGTTCTTCCCAGGACGTCTGCAAGCCCAAGTACAGCGGCACC | SEQ ID NO: 123 |
| [symbol] | NM_ [symbol] | GTTGAAGATGAAACAGTAGAGCTTGATGTGTCAGATGAAGAGATGGCTAGAAGATATGAGACCTTGGTGGGGACAATTGG | SEQ ID NO: 124 |
| NXPH4 | NM_007224 | CTGTGCCAAGCCCTTCAAAGTCATCTGTATCTTCGTCTCTTTCCTCAGCTTTGACTACAAACTGGTGCAGAAGGTGTGCC | SEQ ID NO: 125 |
| HPGD | NM_ [symbol] | CTAATCTTATGAACAGTGGTGTGAGACTGAATGCCATTTGTCCAGGCTTTGTTAACACAGCCATCCTTGAATCAATTGAA | SEQ ID NO: 126 |
| [symbol] | NM_ [symbol] | TTGGATTACAGGAGATGAGAGTATTGTAGGCCTTATGAAGGACATTGTAGGAGCCAATGAGAAAGAAATAGCCCTAATGA | SEQ ID NO: 127 |
| [symbol] | NM_ [symbol] | CAGGCACTGAACAATTTGGGGTTTAAGATTTGTCCCTGTGGCTGGCATCAGTGGAAATGCACCCCCAAGAAATGTTGTTG | SEQ ID NO: 128 |
| KMO | NM_003679 | CATGTCACCACGATCTTTCCTCTGCTTGAGAAGACCATGGAACTGGATAGCTCACTTCCGGAATACAACATGTTTCCCCG | SEQ ID NO: 129 |
| [symbol] | NM_014370 | TGTTCGAGCCGCATTCTGGAGAAGACTACAGTCGTGATGAGGACCACATCGCTCACATAGTGGAGCTTCTGGGGGACATC | SEQ ID NO: 130 |
| THRSP | NM_ [symbol] | CATCACATCCTCATGCACCTCACCGAGAAAGCCCAGGAGGTGACAAGGAAATACCAGGAAATGACGGGACAAGTTTGGTA | SEQ ID NO: 131 |
| PLA2G2A | NM_ [symbol] | TCGCTGCTGTGTCACTCATGACTGTTGCTACAAACGTCTGGAGAAACGTGGATGTGGCACCAAATTTCTGAGCTACAAGT | SEQ ID NO: 132 |
| TFAP2B | NM_ [symbol] | CCTGCACTCCCGAAAGAATATGCTGTTGGCCACCAAGCAACTTTGTAAAGAATTTACGGATCTACTGGCGCAGGACCGGA | SEQ ID NO: 133 |
| FABP7 | NM_001445 | GCACATTCAAGAACACGGAGATTAGTTTCCAGCTGGGAGAAGAGTTTGATGAAACCACTGCAGATGATAGAAACTGTAAG | SEQ ID NO: 134 |
| SLP1 | NM_003064 | GTCCTTCAAAGCTGGAGTCTGTCCTCCTAAGAAATCTGCCCAGTGCCCTTAGATACAAGAAACCTGAGTGCCAGAGTGACT | SEQ ID NO: 135 |
| [symbol] | NM_ [symbol] | ATGAAATGGAGAACTTGCTGACCTACAAGCGGAGAGCCATAGAGCACGTGCTGCAGGTAGAGGCCTCCCAGGAGCCCTCG | SEQ ID NO: 136 |
| S100A9 | NM_ [symbol] | ATGGAGGACCTGGACACAAATGCAGACAAGCAGCTGAGCTTCGAGGAGTTCATCATGCTGATGGCGAGGCTAACCTGGGC | SEQ ID NO: 137 |
| [symbol] | NM_ [symbol] | AGCTTCTCCAGCAGTGCGGGTCCTGGGCTCCTGAAGGCTTATTCCATCCGGACCGCATCCGCCAGTCGCAGGAGTGCCCG | SEQ ID NO: 138 |
| TMEM36A | NM_ [symbol] | AGTGGTGCACTCTTCTTTATCATCTCAGACCTGACCATCGCCCTCAACAAATTCTGTTTTCCTGTGCCCTACTCTCGGGC | SEQ ID NO: 139 |
| MBOAT1 | NM_ [symbol] | CTGTCTCTTACACGGTAGCACCTTTGTGATGTTGGCAGTTGAACCGACCATCAGCTTATACAAGTCCATGTACTTTTAT | SEQ ID NO: 140 |
| PGAP3 | NM_033419 | TCCTGTGCTGCTGTCTGGTTGAGAGCCTGCCACCGTGTGTCGGGAGTGTGGGCCAGGCTGAGTGCATAGGTGACAGGGCC | SEQ ID NO: 141 |
| STARD3 | NM_006804 | CCTGCTCTGGATCATCGAACTGAATACCAACACAGGCATCCGTAAGAACTTGGAGCAGGAGATCATCCAGTACAACTTTA | SEQ ID NO: 142 |
| ERBB2 | NM_004448 | TGATGGGGAGAATGTGAAAATTCCAGTGGCCATCAAAGTGTTGAGGGAAAACACATCCCCCAAAGCCAACAAAGAAATCT | SEQ ID NO: 143 |
| [symbol] | NM_ [symbol] | ATTGAGGCCATCCGAAGAGCCAGTAATGGAGAAACCCTAGAAAAGATCACCAACAGCCGTCCTCCCTGCGTCATCCTGTG | SEQ ID NO: 144 |
| GRB7 | NM_ [symbol] | CTCGATGCACACACTGGTATATCCCATGAAGACCTCATCCAGAACTTCCTGAATGCTGGCAGCTTTCCTGAGATCCAGGG | SEQ ID NO: 145 |
| GSDMB | NM_ [symbol] | CCTGACATGGACTATGACCCTGAGGCACGAATTCTCTGTGCGCTGTATGTTGTTGTTCTCTATATGCTGGAGCTGGCTGA | SEQ ID NO: 146 |
| ORMDL3 | NM_ [symbol] | TCGTGCTGTACTTCCTCACCAGCTTCTACACTAAGTACGACCAGATCCATTTTGTGCTCAACACCGTGTCCCTGATGAGC | SEQ ID NO: 147 |
| MED24 | NM_ [symbol] | ACGATGTGCAGCCTTCGAAGTTGATGCGACTGCTGAGCTCTAATGAGGACGATGCCAACATCCTTTCGAGCCCACAGAC | SEQ ID NO: 148 |
| MSL1 | NM_001012241 | TGTATCACATCACTTCTCAAGTATTCCTTCATTGGGCTTCATCCTTTTAGCAGAACTCTTGGTGGTGGGATAGAGACTTA | SEQ ID NO: 149 |
| [symbol] | NM_007339 | ATGAAGATCGGAAGAATCCAGCATACATACCTCGGAAAGGGCTCTTCTTTGAGCATGATCTTCGAGGGCAAACTCAGGAG | SEQ ID NO: 150 |
| [symbol] | NM_ [symbol] | TGTCCGGTCTTTCTTGGATGATTTTGAGTCAAAGTATTCCTTCCATCCAGTAGAAGACTTTCCTGCTCCAGAAGAATATA | SEQ ID NO: 151 |

[symbol]

indicates data missing or illegible when filed

TABLE 6D

| Symbol | Seequence ID | Sequence of probe | SEQ ID NO: |
|---|---|---|---|
| [symbol] | NM_ [symbol] | [symbol] | SEQ ID NO: 152 |
| MAPT | NM_ [symbol] | [symbol] | SEQ ID NO: 153 |
| [symbol] | NM_ [symbol] | [symbol] | SEQ ID NO: 154 |
| [symbol] | NM_ [symbol] | [symbol] | SEQ ID NO: 155 |
| [symbol] | NM_ [symbol] | [symbol] | SEQ ID NO: 156 |
| [symbol] | NM_ [symbol] | [symbol] | SEQ ID NO: 157 |
| [symbol] | NM_ [symbol] | [symbol] | SEQ ID NO: 158 |
| [symbol] | NM_ [symbol] | [symbol] | SEQ ID NO: 159 |
| STC2 | NM_ [symbol] | [symbol] | SEQ ID NO: 160 |
| [symbol] | NM_ [symbol] | [symbol] | SEQ ID NO: 161 |
| [symbol] | NM_ [symbol] | [symbol] | SEQ ID NO: 162 |
| [symbol] | NM_ [symbol] | [symbol] | SEQ ID NO: 163 |
| [symbol] | NM_ [symbol] | [symbol] | SEQ ID NO: 164 |
| [symbol] | NM_ [symbol] | [symbol] | SEQ ID NO: 165 |
| CHAD | NM_ [symbol] | [symbol] | SEQ ID NO: 166 |
| [symbol] | NM_ [symbol] | [symbol] | SEQ ID NO: 167 |
| [symbol] | NM_ [symbol] | [symbol] | SEQ ID NO: 168 |
| [symbol] | NM_ [symbol] | [symbol] | SEQ ID NO: 169 |
| [symbol] | NM_ [symbol] | [symbol] | SEQ ID NO: 170 |
| [symbol] | NM_ [symbol] | [symbol] | SEQ ID NO: 171 |
| [symbol] | NM_ [symbol] | [symbol] | SEQ ID NO: 172 |
| [symbol] | NM_ [symbol] | [symbol] | SEQ ID NO: 173 |
| NAT1 | NM_ [symbol] | [symbol] | SEQ ID NO: 174 |
| [symbol] | NM_ [symbol] | [symbol] | SEQ ID NO: 175 |
| [symbol] | NM_ [symbol] | [symbol] | SEQ ID NO: 176 |
| [symbol] | NM_ [symbol] | [symbol] | SEQ ID NO: 177 |
| [symbol] | NM_ [symbol] | [symbol] | SEQ ID NO: 178 |
| [symbol] | NM_ [symbol] | [symbol] | SEQ ID NO: 179 |
| [symbol] | NM_ [symbol] | [symbol] | SEQ ID NO: 180 |
| [symbol] | NM_ [symbol] | [symbol] | SEQ ID NO: 181 |
| [symbol] | NM_ [symbol] | [symbol] | SEQ ID NO: 182 |
| [symbol] | NM_ [symbol] | [symbol] | SEQ ID NO: 183 |
| [symbol] | NM_ [symbol] | [symbol] | SEQ ID NO: 184 |
| [symbol] | NM_ [symbol] | [symbol] | SEQ ID NO: 185 |
| [symbol] | NM_ [symbol] | [symbol] | SEQ ID NO: 186 |
| [symbol] | NM_ [symbol] | [symbol] | SEQ ID NO: 187 |
| [symbol] | NM_ [symbol] | [symbol] | SEQ ID NO: 188 |
| [symbol] | NM_ [symbol] | [symbol] | SEQ ID NO: 189 |
| [symbol] | NM_ [symbol] | [symbol] | SEQ ID NO: 190 |
| [symbol] | NM_ [symbol] | [symbol] | SEQ ID NO: 191 |
| [symbol] | NM_ [symbol] | [symbol] | SEQ ID NO: 192 |
| [symbol] | NM_ [symbol] | [symbol] | SEQ ID NO: 193 |
| [symbol] | NM_ [symbol] | [symbol] | SEQ ID NO: 194 |
| [symbol] | NM_ [symbol] | [symbol] | SEQ ID NO: 195 |
| [symbol] | NM_ [symbol] | [symbol] | SEQ ID NO: 196 |
| [symbol] | NM_ [symbol] | [symbol] | SEQ ID NO: 197 |
| [symbol] | NM_ [symbol] | [symbol] | SEQ ID NO: 198 |
| [symbol] | NM_ [symbol] | [symbol] | SEQ ID NO: 199 |
| [symbol] | NM_ [symbol] | [symbol] | SEQ ID NO: 200 |
| [symbol] | NM_ [symbol] | [symbol] | SEQ ID NO: 201 |
| [symbol] | NM_ [symbol] | [symbol] | SEQ ID NO: 202 |
| [symbol] | NM_ [symbol] | [symbol] | SEQ ID NO: 203 |
| [symbol] | NM_ [symbol] | [symbol] | SEQ ID NO: 204 |
| [symbol] | NM_ [symbol] | [symbol] | SEQ ID NO: 205 |
| [symbol] | NM_ [symbol] | [symbol] | SEQ ID NO: 206 |
| [symbol] | NM_ [symbol] | [symbol] | SEQ ID NO: 207 |
| [symbol] | NM_ [symbol] | [symbol] | SEQ ID NO: 208 |
| [symbol] | NM_ [symbol] | [symbol] | SEQ ID NO: 209 |
| [symbol] | NM_ [symbol] | [symbol] | SEQ ID NO: 210 |
| [symbol] | NM_ [symbol] | [symbol] | SEQ ID NO: 211 |
| [symbol] | NM_ [symbol] | [symbol] | SEQ ID NO: 212 |
| [symbol] | NM_ [symbol] | [symbol] | SEQ ID NO: 213 |
| [symbol] | NM_ [symbol] | [symbol] | SEQ ID NO: 214 |
| [symbol] | NM_ [symbol] | [symbol] | SEQ ID NO: 215 |

[symbol]

indicates data missing or illegible when filed

1. A gene expression analysis method for a test sample, comprising the steps of:
(a) measuring an expression level of a desired gene;
(b) measuring an expression level of at least one internal standard gene selected from the group consisting of ABCF3, FBXW5, MLLT1, FAM234A, PITPNM1, WDR1, NDUFS7, and AP2A1; and
(c) normalizing the expression level of the desired gene using the expression level of the internal standard gene.

2. The gene expression analysis method according to claim 1, wherein
the test sample is a sample derived from a breast cancer patient.

3. The gene expression analysis method according to claim 2, wherein
the desired gene is a gene for identifying or classifying a subtype of breast cancer.

4. An internal standard gene for gene expression analysis consisting of at least one gene selected from the group consisting of FBXW5, PITPNM1, MLLT1, WDR1, ABCF3, NDUFS7, FAM234A, and AP2A1.

5. The internal standard gene according to claim 4, wherein
the internal standard gene is used in gene expression analysis for a test sample derived from a breast cancer patient.

6. The internal standard gene according to claim 5, wherein
the gene expression analysis for the test sample derived from a breast cancer patient is gene expression analysis for identifying a subtype of breast cancer.

7. A composition for expression analysis of an internal standard gene, comprising a unit for measuring an expression level of the internal standard gene for gene expression analysis consisting of at least one gene selected from the group consisting of FBXW5, PITPNM1, MLLT1, WDR1, ABCF3, NDUFS7, FAM234A, and AP2A1.

8. The composition for expression analysis of an internal standard gene according to claim 7 for identifying or classifying breast cancer, wherein the internal standard gene is used in gene expression analysis for a test sample derived from a breast cancer patient.

9. The composition for expression analysis of an internal standard gene according to claim 8 for identifying or classifying breast cancer, wherein the gene expression analysis for a test sample derived from a breast cancer patient is gene expression analysis for identifying a subtype of breast cancer.

10. The composition for expression analysis of an internal standard gene for identifying or classifying breast cancer according to claim 8, wherein
the unit for measuring an expression level of the gene is at least one unit selected from the group consisting of a primer and a probe against the gene, and labeled forms thereof.

11. The composition for expression analysis of an internal standard gene for identifying or classifying breast cancer according to claim 9, wherein
the unit for measuring an expression level of the gene is at least one unit selected from the group consisting of a primer and a probe against the gene, and labeled forms thereof.

12. The composition for expression analysis of an internal standard gene for identifying or classifying breast cancer according to claim 8, wherein
the composition is intended for PCR, a microarray, or RNA sequencing.

13. The composition for expression analysis of an internal standard gene for identifying or classifying breast cancer according to claim 9, wherein
the composition is intended for PCR, a microarray, or RNA sequencing.


SEQUENCE LISTING (215 sequences)

SEQ ID NO: 1 - DNA, 2362 bp, Homo sapiens
ctcggccatcgatgcctcggggctcgtggcgtcgggcctggcgggctccccgttgggcgggggcggcggtccgagcggctgcggcagcggaagcgggtccgggtcgcgcagggcgggggcggcgcagcgggcggaggcggccgtgggccgcctgggcagaatgtcacgatggacgagggcggcacgcccctgctccccgacagcctggtctaccagatcttcctgagcctgggcccggccgacgtgctggccgccgggctggtgtgccgccaatggcaggccgtgtcgcgggacgagttcctgtggagggagcagttctaccgctactaccaggtggcccgcgacgtgccccgacacccagcggccatgtcctggtacgaggagttccagcggctgtatgacacggtgccctgcgtggaggtgcagacgctgcgggaacacacagaccaggtcctgcacctcagcttctcccattccgggtaccagttcgcgtcctgctccaaggactgcactgtgaagatctggagcaacgacctgaccatctcgctgctgcacagcgcggacatgcggccctacaactggagctacacccagttctcccagttcaacaaggacgactcgctactgctggcctcgggggtgttcctggggccgcacaactcctcatccggcgagattgctgtcatcagcctagactccttcgcgctgctgtcccgcgtgcggaacaagccctatgacgtgtttggctgttggctcaccgagaccagcctcatctcggggaacctgcaccgcatcggagatatcacctcctgctcggtgctgtggctcaacaatgccttccaggatgtggagtcagagaacgtcaacgtggtgaagcggctgttcaagatccagaacctcaatgccagcaccgtccgcacggtgatggtggccgactgcagccgcttcgacagccctgacctgctgctggaagccggtgacccggccacgtccccctgccgcatctttgacctgggcagcgacaacgaggaggtggtggctggcccggcccccgcccacgccaaggagggcttgcggcactttctggaccgcgtgctggaggggcgggcgcagccacagctgtcggagcgcatgctagagaccaaggtggccgagctgctggcccagggccacaccaagccacccgagcgcagtgccacaggcgccaagagcaagtacctcatcttcaccactggctgcctcacctactccccacaccagatcggcatcaagcagatcctgccacaccagatgaccacggcagggcccgtgctgggtgagggccggggctccgatgccttcttcgacgcgctggaccacgtcatagacatacacggacacatcatcggcatgggcctgtcgcccgacaacaggtacctgtacgtgaacagccgcgcctggcccaacggtgcggtggtggccgaccccatgcagccgccaccaatcgcggaggagattgacctgctggtgttcgacctcaagaccatgcgggaggtgaggcgggctctgcgtgcgcaccgcgcctacacgcccaacgacgagtgcttcttcatcttcctggacgtcagcagggacttcgtggccagcggggcggaggaccggcacggctacatctgggaccgccactacaacatctgtctggccaggctgcggcacgaggatgtggtcaactcagtggtcttcagtccccaggagcaggagctgctgctcacggccagcgacgacgccaccatcaaagcctggcgctccccacgcaccatgcgcgtcctccaggcacctcgcccacggcctcgcaccttcttctcctggcttgccagccagaggcgctgaggtgtgctgggtgcactggagccaccgggaccccttgaggacatcgccaggctctgtggctttttcccgagcgggagaggtggagatgcttatagcagttacgccttaggaaggggacaaccaggccccgccacacgctcacacacaaacctgctcacgcagctgtgatgcttggcacggggtggccagtgcagatggagcccaaggccccctcggcctcctgggcaacttggggtacacaggatactgggggtgccgctcctcactcaaccccaggctaggggtacacctgacccagctggcctcggcccggggcaccttcggctggtcctgtggggccctggacggtggcccagtggtggcaggggctgctcctggctgtggttgtgcgcccggggcttgggagcggccggtcacgctgctgtgggcccgagtgtgttgcatgtccacgcaccacccgttcagggccctgaataaacagttggcaacagca

SEQ ID NO: 2 - DNA, 4217 bp, Homo sapiens
aggggcgccggcgggcggatcgagaggaaggtcggggccatgggccggcactgcgcctcgggagggtccggccaccgctggaacccgaggccggggctgggggcgctccgggctccgacccacgggccggccggccctgcccgggctgggtgaggggcgcccgcctcaagctagaggaggagcggaggccgcgcgcggcccgccgagcgccttcaggatgctcatcaaggaataccacattctgctgcccatgagcctggacgagtaccaggtggcccagctctacatgatccagaaaaagagccgggaggagtctagtggtgagggcagcggcgtggagatcctggccaaccggccctacacggatgggcccgggggcagcgggcaatacacacacaaggtgtaccacgtgggctcccacatcccaggctggttccgggcactgctgcccaaggctgccctgcaggtagaagaggaatcctggaatgcctacccctacacccgaacccggtacacctgccctttcgtggagaaattctccattgaaattgagacctattacctgcctgatggggggcagcagccaaacgtcttcaacctgagcggggccgagaggagacagcgcatcctggacaccatcgacatcgtgcgggatgcagtggccccaggcgagtacaaagcagaagaggacccccggctttatcactcggtcaagacgggccgagggccactgtctgatgactgggcacggacggcggcacagacggggccccttatgtgtgcctataagctgtgcaaggttgagttccgctactggggcatgcaagccaagatcgagcagttcatccatgatgtaggtctgcgtcgggtgatgctgcgggcccaccgccaggcctggtgctggcaggatgagtggacagagctgagcatggctgacatccgggcactggaagaggagactgctcgcatgctggcccagcgcatggccaagtgcaacacaggcagtgaggggtccgaggcccagccccccgggaaaccgagcaccgaggcccggtctgcggccagcaacactggcacccccgatgggcctgaggcccccccaggcccagatgcctcccccgatgccagctttgggaagcagtggtcctcatcctcccgttcctcctactcatcccaacatggaggggctgtgtctccccagagcttgtctgagtggcgcatgcagaacattgcccgagactctgagaacagctccgaggaagagttctttgatgcccacgaaggcttctcggacagtgaggaggtcttccccaaggagatgaccaagtggaactccaatgacttcattgatgcctttgcctccccagtggaggcagagggaacgccagagcctggagccgaggcagctaaaggcattgaggatggggcccaagcacccagggactcagagggcctggatggagccggggagctgggggctgaggcatgcgcagtccacgccctcttccttatcctgcacagcggcaacatcctggactcaggccctggagacgccaactccaagcaggcggatgtgcagacgctgagctccgccttcgaggccgtcacccgcatccacttccctgaggccttgggccacgtggcgctgcgactggtgccctgtccacccatctgcgccgccgcctatgcccttgtctccaacctgagcccttacagccacgatggggacagcctgtctcgctcccaagaccacattccactggctgccctgccactgctggccacctcatcctcccgctaccagggcgccgtggccaccgtcattgcccgcaccaaccaggcctactcagccttcctgcgctcacctgagggtgccggcttctgtgggcaggtcgcactgattggagatggtgttggtggcatcctgggctttgatgcactctgccacagtgctaacgcgggcaccgggagtcggggcagcagccgccgtgggagcatgaacaatgagctgctctctccggagtttggcccagtgcgggaccccctggcagatggtgtggaaggcctgggtcggggcagcccagaaccctcggccttgcctccccagcgcatccccagcgacatggccagtcctgagcccgagggctctcagaacagccttcaggcagcccccgcaaccacctcctcctgggagccccggcgggcaagcacggccttctgcccacccgctgccagttccgaggcacctgacggccccagcagcactgcccgccttgacttcaaggtctctggcttcttcctcttcggctccccactgggcctggtgctggctctgcgcaaaactgtgatgcccgccctggaggcagcccagatgcgcccagcctgtgaacagatctacaacctcttccacgcggctgacccctgcgcctcacgcctcgagcccctgctggccccgaagttccaggccatcgccccactgaccgtgccccgctaccagaagttccccctgggagatggctcatccctgctgctggccgacactctgcagacgcactccagcctctttctggaggagctggagatgctggtgccctcaacacccacctctactagcggtgccttctggaagggcagtgagttggccactgaccccccggcccagccagccgcccccagcaccaccagtgaggtggttaagatcctggagcgctggtgggggaccaagcggatcgactactcgctgtactgccccgaggcgctcaccgcctttcccaccgtcacgctgccccacctcttccacgccagctactgggagtccgccgacgtggtggcgttcatcctgcgccaggtgatcgagaaggagcggccacagctggcggaatgcgaggagccgtccatctacagcccggccttccccagggagaagtggcagcgaaaacgcacgcaggtcaagatccggaacgtcacttccaaccaccgggcgagcgacacggtggtgtgcgagggccgcccccaggtgctaagcgggcgcttcatgtacgggcccctggacgtcgtcacgctcactggagagaaggtggatgtctacatcatgacgcagccgctgtcgggcaagtggatccactttggcaccgaagtcaccaatagctcgggccgcctcaccttcccagttcccccagaacgcgcgctgggcattggtgtctaccccgtgcgcatggtggtcaggggcgaccacacctatgccgaatgctgcctgactgtggtggcccgcggcacggaggctgtggtcttcagcatcgacggctccttcaccgccagcgtctccatcatgggcagcgaccccaaggtgcgagctggcgccgtggacgtggtcaggcactggcaggactccggctacctgatcgtgtatgtcacaggccggccggatatgcagaagcaccgcgtggtggcatggctgtcgcagcacaacttcccccacggcgtcgtctccttctgcgacggcctcacccacgacccactacgccagaaggcaatgtttctgcagagcctggtgcaggaggtagaactgaacatcgtggccggttatgggtctcccaaagatgtggctgtatacgcggcgctggggctgtccccgagccagacctacatcgtgggccgtgccgtgcggaagctacaggcgcagtgccagttcctgtcagacggctatgtggcccacctgggccagctggaagcgggctcgcactcgcatgcctcctcgggacccccgagagctgccttgggcaagagcagctatggtgtggctgcccccgtggacttcctgcgcaaacagagccagctgcttcgctcgaggggccccagccaggcggagcgtgagggcccgggaacaccacccaccaccctggcacggggcaaagcacggagcatcagcctgaagctggacagcgaggagtgaggcccacaccagcctggacctgggttatttattgacacacccaaggggcccgaggggctgcgtgtgggaggctggggacccagacttttggccccagcgctggcccccccagccccacaccctatatctccgtgtgctcctcggtgttacttccctttcatatgaggggacccagcgccggggggagggaggagggcgtgggcatgggcgcagaggcttttccagtgtgtataaatccatgaaaataaacgccacctgcacccca

SEQ ID NO: 3 - DNA, 4532 bp, Homo sapiens
gcgcgacccgggctggcggtggtttccggttccgcggggctggcgggcgagcgggcggccgggatcggcggcggcggcggcggcggcggcgcttgacagacaatgagggcggcggggcggcgttgagcggcggcggcggcggcggcggcggcgagcgacgcggggcccgggggcggggcggggcgccagccatggacaatcagtgcaccgtccaggtgaggttagagctggggcatcgcgcccaactgcgcaagaagcccaccacggaggggttcactcacgactggatggtgtttgtccgcggccccgagcaatgtgacatccagcacttcgtggagaaggtggtcttctggctgcacgacagcttccccaagcccagacgcgtgtgcaaggagcccccctacaaagtagaggagtcggggtacgctggcttcatcatgcccatcgaggtgcacttcaaaaacaaggaggagccgaggaaggtctgcttcacctacgacctgttcctgaacctggaaggcaacccgcccgtgaaccacctgcgctgcgagaagctcaccttcaacaaccccaccacggagttccggtacaagctcctgcgggccggcggggtgatggtaatgcccgaaggagcagacacggtgtccaggcccagtcccgactaccccatgttacccacaattccactctctgccttctctgaccccaagaagaccaaaccatcccacggctccaaggacgccaacaaggagagcagcaagacctccaagccacacaaggtgaccaaggagcaccgggagcggccccgcaaagactccgagagcaagagctcctccaaggagctggagcgtgagcaggccaaaagctccaaggacacctcgcggaagctgggcgagggccggctgcccaaggaggagaaggcgccaccgcccaaggctgccttcaaggaacccaagatggccctgaaagagaccaagctggaaagcacgtcccccaagggtgggcccccacccccacccccacccccaccccgggcttccagcaagcggccggccaccgccgactcgccaaagcccagcgccaagaagcagaagaagagcagctcgaaggggtcccggagtgctccaggcacctcgccccgcacctcctcctcctcctccttctcggacaagaagccggccaaggacaagagcagcaccagaggggagaaggtgaaggccgagagtgagccccgggaggccaaaaaggccctggaggtggaggagtccaactcagaggacgaggcctccttcaagtccgagtctgcccagtcaagcccgtccaactccagctccagctcagactccagctcagactcagacttcgagccatcccagaaccacagccaaggacccctgcgctccatggtggaggacctgcagtccgaggagtccgacgaggacgactcttcgtcaggcgaggaggctgccggcaagaccaacccggggagggactccaggttgagcttcagcgacagcgagagtgacaacagcgccgactcctccctgcccagccgtgagcccccacccccccagaagccacccccgcccaacagcaaggtgtcaggccggaggagccccgagtcctgcagcaagcctgagaagatcctcaagaagggcacctacgacaaggcctacacggatgagctggtggagctacaccggaggctgatggcgctgcgggagcgcaacgtgctgcagcagattgtgaatctgatcgaggagactggccacttcaatgtcaccaacaccaccttcgacttcgacctcttctccctggacgagaccaccgtgcgcaaactgcagagctgcctggaggccgtggccacatgaccctgggccgcgtgccgggcccccaccgccggggtcctgggaggctgcagccaggcctcgcctgccctcctctcccgtctcgcctgcccgcagcactgccttagcgacagccctgtcctcgcccgcacagcccgctgcactttcctcggcggctccctgcccactctccccgctccatcgccaggagccctggggccgggctgggccacacgggagccgccccccgggctctctgggctcccctcagagagcggctggtgttgaaggcagctctgggcagagacagggccgggggcccctcgcccagccttgggaccagcccccactgctcggcgggctgtgcggcgtcgctgccccagcctttctgagcagcggatcgacggctggggggcatctccagcctcccgaatggttcagaacacaccctccgcccaccctggacctcagaaggccctgcctgtgtgtctgcgtcgtcctccgtgtagacctagcgccggctcctccccggctcgcgctgcgctggggtctgtgctgtatagttctctctattatatatgtgtgtgcactgtctggtcaagagcagtgtccatgtgcgtttctgtggcagcagtgcacgtggggtgggtggggggtgcgtacggtggggcttgtaggttaagatgtctctgcggtggtgtggggctggaggccgagccaggcacggcagctggccctctccaggcgcatggaggaattctccctttgccgttccaaggatgccgccgcctcctaccggggctcccgggggggtcagccaacactacaaagggcagcgagtcctcagggcgcccgggagccaccagtctgctctcgggctctactcagactagcgccaacagtctccgcggacagactcgcatcagccgccctggggctcggcctcctcccataggctcctcttcctctctttcattcaagtcttcaaactttgaaaaaaatatgagcttttgccaaataagacgggatagtttatggagttttctgaggcgcattgaactctggtcagcccctcgagcgtgggctcggccatcagcaggcccggtgggttggccgggctggccctcccaggctgcctttctctctggtcgcggcctggcccgccccggcctccaccgccgcaattcatgctggtgccgcgcccgcagccccactcctggtatgcctgagattctccaggggccaagccggtcctccttcccggcccactcttccagggccaggacgctcccgcggagccagcccatcgtggctgggggtcccaggggctggaggcctgcctggttagcctctgtttcccagacattgactcgaggcgcctccagtcctctcaaccccctgactttcaagtcctttcaagtccttgtgccctgaccaagttatgaggaaaagagggctgagggcctggaggtgggctcgcaccccctcgaacccacctagatgtctcctcctccccagcctatgagttcagacttggggggctccatctgtccctccctgaggtccctcatcttgtccctgctctgattctgtcagcagatcctgaacactccttggtggggacccatgcctccaaggctgcccccaatgccagctgtcctggggaaactccccagaactctcggggaaaggcccgagaaccttcggaggaagcctccgggactcctcgccacctgctctgtttctctcccggccccactgccttcaggtatcaggaagagggcaggcccctgtgccccctctccccttcccagggcggccaacaatactccaccatggggcgcttcctttcttctctggaaactaagacattggactgaaggaactggcctgaggggggctgaccgggggcccgtgctgctgggagctctacgcacaagaggccgccggggtcacggacaggaacctcagcccagggtgctggcctttctccttcgcaggagtcccccaaaatccacacagcgactcgagcagccttccactcagccagaggagggtcgggctcccccgcctgctctcggtggtggcgtctggcccttcctgccgtttccaaggcctccggaagcaccaacccagttctcagttacctcctctggccggaaggcctcacgttgtcctccagagctgctctgggcccagggctggctccgtgaccacctgatggacgtgctgggcagcatgggctcccaaggcagcatccccagctgctctcccaccccaccccagctgcccggcggctgggcccacgagggctctggggacacttccataggaaaggggcctggcactttgtgattgccacgtgtttcctgttaagccgcctgcccccagtgcaaatctctgtgttttgctctctcctgaacaaaaatgtaaaccgacgccggaaagcaaatggtatcgaatacctttcttgcccataagggtttttacaaagaacgtgtctcactaggaactaggacactcatcggcccgagaccaactcctggataaaaggaataaggagaatcgtgtttgtaacgagttacaggattgtttctttcctggattttaaacttttgttaaattgtgaaattatggaggagttttatagaaaaaaaaagtgaaataaagtcagatgggaaagca

SEQ ID NO: 4 - DNA, 2993 bp, Homo sapiens
actccactcccggcacgcccggtgccgccttccggctccagtccccgggctcggcctcggcgaggtgtaattcgcagcgcgggccggccccggaggctctcggcgagcgcggcgcggtaacaagtgggcgaggatgccgtacgagatcaagaaggtgttcgccagcctcccgcaggtggagaggggcgtctccaagatcatcggcggcgaccctaagggcaacaattttctgtacaccaatggaaagtgcgtcatcctaaggaacatcgacaacccagcccttgctgacatctacacagagcacgcccatcaggtggtggtggccaagtatgcgcccagcggattctacattgcctccggagatgtgtctgggaagctgaggatctgggataccacgcagaaggagcacctgttgaagtatgagtaccagcctttcgctgggaagatcaaagacattgcttggactgaagacagtaagaggatcgccgtggtcggggaaggaagggagaagtttggagcagtcttcctctgggatagtggctcttctgtgggcgagattacaggacacaacaaagtcatcaacagcgtggacatcaagcagagccggccataccggctggccacgggaagcgatgataactgcgcggcattctttgagggacccccattcaagttcaagttcacaattggcgaccacagccgctttgtcaactgtgtgcgattctctcctgatgggaacagatttgccacagccagtgctgacggccagatatacatctatgacgggaagactggggagaaggtgtgcgcgctgggcggaagcaaggcccacgacggtgggatttacgcaattagttggagtcccgacagcacccatttgctttctgcttctggggacaaaacttccaagatttgggacgtcagcgtgaactccgtggtcagcacatttcccatgggctccacggttctggaccagcagctgggctgcctatggcagaaggaccacctgctcagtgtctccctgtccgggtacatcaactatctggacagaaacaaccccagcaagcccctgcacgtcatcaagggtcacagtaaatcgatccagtgtctgacggtgcataaaaacggcggcaagtcctacatttactctgggagccacgacggacacattaattactgggattcagagacgggggagaacgactccttcgctgggaaaggccacacgaaccaggtgtccaggatgaccgtggatgagtcggggcagctcatcagctgcagcatggacgacaccgtgcggtacaccagcctcatgctgcgggactacagcggacaaggagttgtgaaactggacgttcagccaaagtgcgtagccgtcggccccgggggatacgccgtggtcgtgtgcattggacagattgtcctgctgaaggatcagaggaagtgcttcagcatcgacaaccccggctacgagcccgaagttgtggcagtgcaccccggcggggacacggtggcaattgggggtgtggacggcaacgtccgcctgtattccatcctgggcaccacgctgaaggatgagggcaagctcctagaggccaagggccccgtgaccgacgtggcctactcccacgacggcgccttcctcgcggtgtgcgacgccagcaaggtggtcacagtgttcagcgttgctgacggctactcggagaacaatgttttttatggacaccatgcaaaaatcgtctgcctggcctggtccccagacaatgaacactttgcctccggtggcatggacatgatggtgtatgtttggaccctgagtgacccggaaaccagagtcaagatccaagatgcacaccggctgcaccatgtcagcagcctggcctggctggacgagcacacgctggtcacgacctcccatgatgcctctgtcaaggagtggacaatcacctactgaggagccccacccccgcctctggatggaccgaatcagggactagagtttaactgcagcggaacatgtcatttctctatttctgtgacgcgcccccatgcccccaccccaccacaagaggcaggagggcccagtcatgaccctcgtctctgcagggtgtctgtacacgttcttctgaaagctttagacagtaacagtttgcacatgaaaaataaagcgagcacctaaacaatgtgtggagcataactaaaacccacagcccaaccaaaccttgagaatgcgaaacattccagaggcagtagcctccaaagcacacagagcccctggccccgccgcggctctcactatctgtcaggggaggttgtacaggtgaatgagccggggggctcatgttcctgcctgcagaacatttctgtactagtgagaagagggaatatgcattgcagttcagcaaagccggaattctgtgttgaacagatgtctgtctccctagtgtgtgactcacaccttgtggctgccttcagagcgccacctccagatcagatggggacacacaacccctggatatgtttcattgtcagattttgtgcttgattttaagaatggaattgtgggtatctttcctttttttttaatgtatcttaactgttgcctgtcagtgtttacaaactagtgcgttgacggcaccgtgtccaagtttttagaacccttgttagccagaccgaggtgtcctggtcaccgtttcaccatcatgctttgatgttcccctgtctttccctcttctgctctcaagagcaaaggttaatttaaggacaaagatgaagtcactgtaaactaatctgtcattgtttttaccttccttttctttttcagtgcagaaattaaaagtaagtataaagcaccgtgattgggagtgtttttgcgtgtgtcggaatcactggtaaatgttggctgagaacaatccctccccttgcacttgtgaaaacactttgagcgctttaagagattagcctgagaaataattaaatatcttttctcttca

SEQ ID NO: 5 - DNA, 2626 bp, Homo sapiens
gcactccgctcctttcctgcgtcaccgctacacatgcgccttgcaaggaagggcgagatttcgggggcagggcggggggcgggcctaggccggggtctcacagcgactgcgcggacgggttcctgagtggaacatggcgacttgcgccgaaatcctgcggagcgagttccccgaaattgacggacaagtcttcgactacgtgaccggcgtcttgcacagcggcagcgcggacttcgagtctgtggatgacctggtggaagctgtaggggaactattgcaagaggtgtccggggacagcaaggatgacgcgggcatcagggccgtgtgccagcgcatgtacaacactctgcgtctggctgagccacaaagccagggaaatagccaggtgctactggacgcccctatccagttgtcaaagataacggagaactacgactgtggaaccaaacttccaggactgctaaagagggaacagtcctcgacagtgaatgcaaagaagttagagaaggccgaggctcgacttaaggcaaagcaggagaagcgctcagagaaggacacgctcaagaccagcaaccctctagtcttagaagaggcatcagccagccaggcaggcagcagaaaggagagtcggttggaatcatctggcaagaacaaatcctatgatgtgcgaattgagaactttgatgtgtcttttggcgatagagtactgctggctggagcggatgtgaacctggcatggggccgccgttacgggctggtggggcggaatgggttggggaagacaacgttactgaagatgctggccacccggagtctgcgggttccagcccacatttccctgctgcacgttgagcaagaggttgctggagatgacactcctgccctgcagagtgtgctggagagtgacagtgtgcgagaggatttgctacggagggagcgggagctcactgcccagattgctgctggcagggcggagggctctgaagctgcagagctggcagaaatctatgccaaactggaggagattgaggctgacaaggcacctgccagggcatcagtcattctcgctgggcttggctttacccctaaaatgcagcagcagcccacccgggagttctcaggtggctggaggatgaggctggccctggcccgggccctctttgctaggccagatcttctgctgttagatgaacctacaaacatgctggatgtcagggccatcctgtggctggagaattacctgcagacgtggccctccaccatcctagtcgtctcccacgaccgcaacttcttgaatgccatcgccacagacatcatccacctgcacagccagcggctagatggttaccggggagactttgagaccttcatcaagagtaagcaggagcggctgctcaaccagcagcgtgaatatgaggcgcagcagcagtatcgccagcacatccaggttttcattgaccggtttcgctacaatgccaacagggcctctcaagtgcagagtaaactcaagatgctggagaagctgcctgagctgaagcctgtggacaaggaatcagaggtcgtaatgaagttccctgatgggtttgagaagttctcgccgccaattctgcagctagatgaggtggatttctactacgatccgaagcacgtcatcttcagtcgcctctctgtgtctgctgatctcgagtctcgcatctgtgtggttggagagaatggggctgggaagtctaccatgctgaagctgcttttgggggacctggcacctgttcggggcatcagacacgctcacaggaatctgaagattggctatttcagccagcaccatgtggagcagctggacctaaacgtcagtgctgtggaactgctggcacgcaagtttcctgggcggcctgaggaggagtaccgtcaccagctgggtcggtatggcatctccggagaactggccatgcgtcctcttgccagcctgtctgggggccagaagagccgagtggcctttgctcagatgactatgccctgccccaacttctacattctggatgaacccacaaaccacctggacatggagaccattgaggctctgggccgtgccctcaacaatttcaggggtggtgtgattctggtgtcccacgatgagcgctttatcaggctggtgtgccgggagttgtgggtatgcgaaggaggcggcgtcacccgtgtggaaggaggatttgaccagtaccgcgccctcctccaggaacagttccgccgcgaaggcttcctctagggccaccaggctgaggactcgcccaggacatggactggtctctcagacccctgggccaccatgtaggccaccactccaggccgtggacttcccccaacttggggacagccttattcccaaatgtctctatccttttgactggagcatcttctgcacaaccttgggagcccatccaagggttggtgaggactggtctcccgggggtgggggtctggggggtaccctctggggttatagattcccccactgccccagctctgactggaccccaagtggctgctatgtaaattaaatctctccccgcgtctcaaaaaaaaaaaaaaaaaaaaaaaaaaaaaaaaaaaaaaaaaaaaaaaaaaaaaa

SEQ ID NO: 6 - DNA, 758 bp, Homo sapiens
gtctgaaggccgaggccaagatggcggtgctgtcagctcctggcctgcgcggcttccggatccttggtctgcgctccagcgtgggcccggctgtgcaggcacgaggtgtccatcagagcgtggccaccgatggcccaagcagcacccagcctgccctgccaaaggccagagccgtggctcccaaacccagcagccggggcgagtatgtggtggccaagctggatgacctcgtcaactgggcccgccggagttctctgtggcccatgaccttcggcctggcctgctgcgccgtggagatgatgcacatggcagcaccccgctacgacatggaccgctttggcgtggtcttccgcgccagcccgcgccagtccgacgtcatgatcgtggccggcacactcaccaacaagatggccccagcgcttcgcaaggtctacgaccagatgccggagccgcgctacgtggtctccatggggagctgcgccaacggaggaggctactaccactattcctactcggtggtgaggggctgcgaccgcatcgtgcccgtggacatctacatcccaggctgcccacctacggccgaggccctgctctacggcatcctgcagctgcagaggaagatcaagcgggagcggaggctgcagatctggtaccgcaggtagcgccgccgccgccgccgccggagcctgtcgccgtcctgtccccagcctgcttgtgtcccgtgaggttgtcaataaacctgccctcgggctgccgcc

SEQ ID NO: 7 - DNA, 2909 bp, Homo sapiens
gccagcggcgcggtcgggtgagaggccgcggcggcaggtccacctgggcttgcgaaggcacagattccccgtccacagctcacgaccagatgcaccagcaggagtccacatcgaggacgtcctccgggcactcccacgaccagtgaccaggagttaaactttgggatgtgcccgtgatgttggaccacaaggacttagaggccgaaatccaccccttgaaaaatgaagaaagaaaatcgcaggaaaatctgggaaatccatcaaaaaatgaggataacgtgaaaagcgcgcctccacagtcccggctctcccggtgccgagcggcggcgttttttctttcattgtttctctgcctttttgtggtgttcgtcgtctcattcgtcatcccgtgtccagaccggccggcgtcacagcgaatgtggaggatagactacagtgccgctgttatctatgactttctggctgtggatgatataaacggggacaggatccaagatgttctttttctttataaaaacaccaacagcagcaacaatttcagccgatcctgtgtggacgaaggcttttcctctccctgcacctttgcagctgctgtgtcgggggccaacggcagcacgctctgggagagacctgtggcccaagacgtggccctcgtggagtgtgctgtgccccagccaagaggcagtgaggcaccttctgcctgcatcctggtgggcagacccagttctttcattgcagtcaacttgttcacaggggaaaccctgtggaaccacagcagcagcttcagcgggaatgcgtccatcctgagccctctgctgcaggtgcctgatgtggacggcgatggggccccagacctgctggttctcacccaggagcgggaggaggttagtggccacctctactccggcagcaccgggcaccagattggcctcagaggcagccttggtgtggacggggaaagtggcttcctccttcacgtcaccaggacaggtgcccactacatcctctttccctgcgcaagctccctctgcggctgctctgtgaagggtctctacgagaaggtgaccgggagcggcggcccgttcaagagtgacccgcactgggagagcatgctcaatgccaccacccgcaggatgctttcccacagctctggagcagtgcgctacctgatgcatgtcccagggaacgccggtgcagatgtgcttcttgtgggctcagaggccttcgtgctgctggacgggcaggagctgacgcctcgctggacacccaaggcagcccatgtcctgagaaaacccatcttcggccgctacaaaccagacaccttggctgtagccgttgaaaacggaactggcaccgacagacagatcctgtttctggaccttggcactggagccgtcctgtgtagcctagccctcccgagcctccctgggggtccactgtccgccagcctgccgaccgcagaccaccgctcagccttcttcttctggggcctccacgagctggggagcaccagcgagacggagaccggggaggcccggcacagcctgtacatgttccaccccaccctgccgcgcgtgctgctggagctggccaatgtctctacccacattgtcgcctttgacgccgtcctgtttgagccaagccgccacgccgcctacatccttctgacaggcccggcagactcagaggcacccggcctggtctctgtgatcaagcacaaggtgcgggaccttgtcccaagcagcagggtggtccgcctgggtgagggtgggccagacagtgaccaagccatcagggaccggttctcccggctgcggtaccagagtgaggcgtagaggcacgccagccagagcctgtggagagactccgcctgctgacactaaacgtcctgggaagtgggcccttccctgggtctctgcactgactcccccactcctgaccctggtgatggtcgccactgggcagcagcagccttaccagtcctccatgatcacacccagggacctgcatgggtgaggggacaccctgggcctctctcccgcccagcatcctccctgagtccccacacagggcctcactctgcaccccaccagggtcccgctcacaccaggcagccttcatagtggtctccctggccaccttgggcagagctgggtcatgcagcaccccatccttacccggtgccctctccttgccagcttctccccaggccagagcggccatcgcgtagaaagaaccagggtgtccccgggacaggccgtcccccaccccatcctgtagaagtccattccccttttccctcctgtgctctgtcccccaaggagtcatggaactcagggtactgggcctcaacgggaacctgagacagctccagcttcgcagcccttcccggagctacagggggatcctctagcatggggggtgtgacttggttcctttgaccaggtcctgtgaggaagcctggagcaagggtctcccccagcaggatgggtggggcctgctctggagctgagcccgtggccgctcacaggtgtccttagtggtgttgcagctgtctactggctgcatgtgctgtgaatatcccaaggaactggctgtggaatgcgtgtttgggtcagtctgtgccctctcagtagacactggagctgctctgtccctgaagaggccccgtgccccaggcatggcaagcgcctgcctctccccttccggtgctcacacgcccacgccgtgccacccgatgcaggactcacctctgtgccttgctgctcctgaggcccaagggcagccatggtgctctgtactgctcgggccgcccaggtcacagagcctgagcttcgtagccaaagcagcctgatgacccacccaccaaggaagaaagcagaataaacatttttgcactgcctgaaaaa

SEQ ID NO: 8 - DNA, 3433 bp, Homo sapiens
cggctcagagctccggaccgcgggcggaggggaggggcagggggcggtgccacggcctgccagcccgcccgcccgcccgccagccagccctccccgcggccggctcggctccttggcgctgcctggggtcctttccgcccggtccccgcttgccagcccccgctgctctgtgccctgtccggccaggcctggagccgacaccaccgccatcatgccggccgtgtccaagggcgatgggatgcgggggctcgcggtgttcatctccgacatccggaactgtaagagcaaagaggcggaaattaagagaatcaacaaggaactggccaacatccgctccaagttcaaaggagacaaagccttggatggctacagtaagaaaaaatatgtgtgtaaactgcttttcatcttcctgcttggccatgacattgactttgggcacatggaggctgtgaatctgttgagttccaataaatacacagagaagcaaataggttacctgttcatttctgtgctggtgaactcgaactcggagctgatccgcctcatcaacaacgccatcaagaatgacctggccagccgcaaccccaccttcatgtgcctggccctgcactgcatcgccaacgtgggcagccgggagatgggcgaggcctttgccgctgacatcccccgcatcctggtggccggggacagcatggacagtgtcaagcagagtgcggccctgtgcctccttcgactgtacaaggcctcgcctgacctggtgcccatgggcgagtggacggcgcgtgtggtacacctgctcaatgaccagcacatgggtgtggtcacggccgccgtcagcctcatcacctgtctctgcaagaagaacccagatgacttcaagacgtgcgtctctctggctgtgtcgcgcctgagccggatcgtctcctctgcctccaccgacctccaggactacacctactacttcgtcccagcaccctggctctcggtgaagctcctgcggctgctgcagtgctacccgcctccagaggatgcggctgtgaaggggcggctggtggaatgtctggagactgtgctcaacaaggcccaggagccccccaaatccaagaaggtgcagcattccaacgccaagaacgccatcctcttcgagaccatcagcctcatcatccactatgacagtgagcccaacctcctggttcgggcctgcaaccagctgggccagttcctgcagcaccgggagaccaacctgcgctacctggccctggagagcatgtgcacgctggccagctccgagttctcccatgaagccgtcaagacgcacattgacaccgtcatcaatgccctcaagacggagcgggacgtcagcgtgcggcagcgggcggctgacctcctctacgccatgtgtgaccggagcaatgccaagcagatcgtgtcggagatgctgcggtacctggagacggcagactacgccatccgcgaggagatcgtcctgaaggtggccatcctggccgagaagtacgccgtggactacagctggtacgtggacaccatcctcaacctcatccgcattgcgggcgactacgtgagtgaggaggtgtggtaccgtgtgctacagatcgtcaccaaccgtgatgacgtccagggctatgccgccaagaccgtctttgaggcgctccaggcccctgcctgtcacgagaacatggtgaaggttggcggctacatccttggggagtttgggaacctgattgctggggacccccgctccagccccccagtgcagttctccctgctccactccaagttccatctgtgcagcgtggccacgcgggcgctgctgctgtccacctacatcaagttcatcaacctcttccccgagaccaaggccaccatccagggcgtcctgcgggccggctcccagctgcgcaatgctgacgtggagctgcagcagcgagccgtggagtacctcaccctcagctcagtggccagcaccgacgtcctggccacggtgctggaggagatgccgcccttccccgagcgcgagtcgtccatcctggccaagctgaaacgcaagaaggggccaggggccggcagcgccctggacgatggccggagggaccccagcagcaacgacatcaacgggggcatggagcccacccccagcactgtgtcgacgccctcgccctccgccgacctcctggggctgcgggcagcccctcccccggcagcacccccggcttctgcaggagcagggaaccttctggtggacgtcttcgatggcccggccgcccagcccagcctggggcccacccccgaggaggccttcctcagcccaggtcctgaggacatcggccctcccattccggaagccgatgagttgctgaataagtttgtgtgtaagaacaacggggtcctgttcgagaaccagctgctgcagatcggagtcaagtcagagttccgacagaacctgggccgcatgtatctcttctatggcaacaagacctcggtgcagttccagaatttctcacccactgtggttcacccgggagacctccagactcagctggctgtgcagaccaagcgcgtggcggcgcaggtggacggcggcgcgcaggtgcagcaggtgctcaatatcgagtgcctgcgggacttcctgacgcccccgctgctgtccgtgcgcttccggtacggtggcgccccccaggccctcaccctgaagctcccagtgaccatcaacaagttcttccagcccaccgagatggcggcccaggatttcttccagcgctggaagcagctgagcctccctcaacaggaggcgcagaaaatcttcaaagccaaccaccccatggacgcagaagttactaaggccaagcttctggggtttggctctgctctcctggacaatgtggaccccaaccctgagaacttcgtgggggcggggatcatccagactaaagccctgcaggtgggctgtctgcttcggctggagcccaatgcccaggcccagatgtaccggctgaccctgcgcaccagcaaggagcccgtctcccgtcacctgtgtgagctgctggcacagcagttctgagccctggactctgccccgggggatgtggccggcactgggcagccccttggactgaggcagttttggtggatgggggacctccactggtgacagagaagacaccagggtttgggggatgcctgggactttcctccggccttttgtatttttatttttgttcatctgctgctgtttacattctggggggttagggggagtccccctccctccctttcccccccaagcacagaggggagaggggccagggaagtggatgtctcctcccctcccaccccaccctgttgtagcccctcctaccccctccccatccaggggctgtgtattattgtgagcgaataaacagagagacgctaa

SEQ ID NO: 9 - DNA, 80 bp, Artificial sequence
  chemically synthesized
accactggctgcctcacctactccccacaccagatcggcatcaagcagatcctgccacaccagatgaccacggcagggcc

SEQ ID NO: 10 - DNA, 80 bp, Artificial sequence
  chemically synthesized
cactccagcctctttctggaggagctggagatgctggtgccctcaacacccacctctactagcggtgccttctggaaggg

SEQ ID NO: 11 - DNA, 80 bp, Artificial sequence
  chemically synthesized
atctgatcgaggagactggccacttcaatgtcaccaacaccaccttcgacttcgacctcttctccctggacgagaccacc

SEQ ID NO: 12 - DNA, 80 bp, Artificial sequence
  chemically synthesized
agcctggcctggctggacgagcacacgctggtcacgacctcccatgatgcctctgtcaaggagtggacaatcacctactg

SEQ ID NO: 13 - DNA, 80 bp, Artificial sequence
  chemically synthesized
tgactatgccctgccccaacttctacattctggatgaacccacaaaccacctggacatggagaccattgaggctctgggc

SEQ ID NO: 14 - DNA, 80 bp, Artificial sequence
  chemically synthesized
actattcctactcggtggtgaggggctgcgaccgcatcgtgcccgtggacatctacatcccaggctgcccacctacggcc

SEQ ID NO: 15 - DNA, 80 bp, Artificial sequence
  chemically synthesized
tggcaccgacagacagatcctgtttctggaccttggcactggagccgtcctgtgtagcctagccctcccgagcctccctg

SEQ ID NO: 16 - DNA, 80 bp, Artificial sequence
  chemically synthesized
agcattccaacgccaagaacgccatcctcttcgagaccatcagcctcatcatccactatgacagtgagcccaacctcctg

SEQ ID NO: 17 - DNA, 80 bp, Artificial sequence
  chemically synthesized
ctttgagtctatcaaaaggaaacttcctttcctcaactgggatgcctttcctaagctgaaaggactgaggagcgcaactc

SEQ ID NO: 18 - DNA, 80 bp, Artificial sequence
  chemically synthesized
tggaagagagctatgacctcaaggacacgttgagaaccatgggaatggtggatatcttcaatggggatgcagacctctca

SEQ ID NO: 19 - DNA, 80 bp, Artificial sequence
  chemically synthesized
cctcagcagtgccagcagaaatatcctcctgtgacaccttccccaccctgccagtcaaagtatccacccaagagcaagta

SEQ ID NO: 20 - DNA, 80 bp, Artificial sequence
  chemically synthesized
gttttcagctgctcagaattcatctgaagagagacttaagatgaaagcaaatgattcagctcccttatacccccattaaa

SEQ ID NO: 21 - DNA, 80 bp, Artificial sequence
  chemically synthesized
ccgtgtctcaagatacgtcctgtggatccgtgaaacaatccgaaaatatgaaacccagcagcaaaaatggttgaagggcc

SEQ ID NO: 22 - DNA, 80 bp, Artificial sequence
  chemically synthesized
tccgaagaagagtggaccaactgaagagtgatcaatctcggttggattcggaactgaagaacatgcaggacatggtggag

SEQ ID NO: 23 - DNA, 80 bp, Artificial sequence
  chemically synthesized
gcagcccttcgaggtgctcatcatcgcgtcagacgacggcttcaaggccgtggttggggacgcccagtaccaccacttcc

SEQ ID NO: 24 - DNA, 80 bp, Artificial sequence
  chemically synthesized
gttcctgttaaaggtcaagacactgtcaaaggccgtgttccattcaatggacaagatcccgttaaaggacaagtttcagt

SEQ ID NO: 25 - DNA, 80 bp, Artificial sequence
  chemically synthesized
cccaagctgttctacgccgaccaccccttcatcttcctagtgcgggacacccaaagcggctccctgctattcattgggcg

SEQ ID NO: 26 - DNA, 80 bp, Artificial sequence
  chemically synthesized
gctccttcctggtcaagaagcatttcaacgcctccaaaaagccaaactacagcgaactggacacacatacagtgattatt

SEQ ID NO: 27 - DNA, 80 bp, Artificial sequence
  chemically synthesized
cacggctcttcatggaccttcagtgcactcagctgcaagattgtggcctttatggccgtgctcttttgcttccatgcggc

SEQ ID NO: 28 - DNA, 80 bp, Artificial sequence
  chemically synthesized
cagacagttctaattgttggaacgttgctctatgcatgctattgggtcatgcttttgacgctgtatgtagttctcatcaa

SEQ ID NO: 29 - DNA, 80 bp, Artificial sequence
  chemically synthesized
ttttgtcgcaatcatatactgtttctgcaatggcgaggtacaagctgagatcaagaaatcttggagccgctggacactgg

SEQ ID NO: 30 - DNA, 80 bp, Artificial sequence
  chemically synthesized
tgatgtggaagcttttcaacaggaactggctctgcttaagatagaggatgcacctggagatggtcctgatgtcagggagg

SEQ ID NO: 31 - DNA, 80 bp, Artificial sequence
  chemically synthesized
tgcatttgatggcctgtattttctccgcactgagaatggtgttatctaccagaccttctgtgacatgacctctgggggtg

SEQ ID NO: 32 - DNA, 80 bp, Artificial sequence
  chemically synthesized
agatgccactccccagaatcccttcttgaagtccagacctcaggttaggccaaaaccagctccttcccccaaaacggagc

SEQ ID NO: 33 - DNA, 80 bp, Artificial sequence
  chemically synthesized
acccagtggtctgttgactcccttacacttggagggccttgtccagttccaagatgtctcctttgcctacccaaaccgcc

SEQ ID NO: 34 - DNA, 80 bp, Artificial sequence
  chemically synthesized
aagacataccacgtaggagaacagtggcagaaggaatatctcggtgccatttgctcctgcacatgctttggaggccagcg

SEQ ID NO: 35 - DNA, 80 bp, Artificial sequence
  chemically synthesized
gaaagctttgaggagtcctggacacccaactacaagcagtgttcatggagttcattgaattatggcatagatcttgggaa

SEQ ID NO: 36 - DNA, 80 bp, Artificial sequence
  chemically synthesized
gtgagttcccggagtgagttgaaccaggtggaccaagtgggctacgtgacctatgacatcctgcagtgccctgaggacta

SEQ ID NO: 37 - DNA, 80 bp, Artificial sequence
  chemically synthesized
ctatatgaaggatgtgaaggtcagcctcttcaagaaggacaaggctatgctcttcacctatgatcagtaccaggaaaata

SEQ ID NO: 38 - DNA, 80 bp, Artificial sequence
  chemically synthesized
gctgtcattggtgctgtcctggctgtgtttggaggtattctaatgccagttggagacctgcttatccagaagacaattaa

SEQ ID NO: 39 - DNA, 80 bp, Artificial sequence
  chemically synthesized
ggaaacggtccaggagctgatccccctggccaaggagatgatggcccagaagcgcaaggggaagatggtgaagctgtacg

SEQ ID NO: 40 - DNA, 80 bp, Artificial sequence
  chemically synthesized
gaagacattggaggcaaaaagctgactgagatcatcaacacgcagcatgagaatgtcaaatacctgccagggcacaagtt

SEQ ID NO: 41 - DNA, 80 bp, Artificial sequence
  chemically synthesized
acactggcagtctaccaacagatcctcaccagtatgccttccagaaacgtgatccaaatatccaacgacctggagaacct

SEQ ID NO: 42 - DNA, 80 bp, Artificial sequence
  chemically synthesized
ccactgggcacggattccctcaagaacctgaccctgagggacttgagcctgaggggaaactccgagacgtcgtcggacac

SEQ ID NO: 43 - DNA, 80 bp, Artificial sequence
  chemically synthesized
ctgggtggtgtggcacataccctgcagaagaccctccagaccaccatctcggctgtgacatgggcacctgcagctgtgct

SEQ ID NO: 44 - DNA, 80 bp, Artificial sequence
  chemically synthesized
cctgcctagcagtgagcagatgccaaatgaccaggaagaggagtcctttgcagagggtcattccgaagcgtccctcgcca

SEQ ID NO: 45 - DNA, 80 bp, Artificial sequence
  chemically synthesized
atttagtgtctccagactctcctagatccatagacagcaacagtgagattgtctcatttggaagtccatgctccattaat

SEQ ID NO: 46 - DNA, 80 bp, Artificial sequence
  chemically synthesized
ggataagaagcgattcagctttgtcttcaagcctgtcttcaacacctcactgctctttctacagtgtgagctgacgctgt

SEQ ID NO: 47 - DNA, 80 bp, Artificial sequence
  chemically synthesized
aagggtggtccaactgccaaagtcaagcaaggccacatcagcttcaaggttatttccagcgatgatctcactgagctcta

SEQ ID NO: 48 - DNA, 80 bp, Artificial sequence
  chemically synthesized
caagaaggcaaaaaggtcatccaaggaggaagccgagatggcctacaaagacttcctgctccagtccagcaccgtggccg

SEQ ID NO: 49 - DNA, 80 bp, Artificial sequence
  chemically synthesized
ggtagcagcagcaatttccacatcaatgtagaagagtcagtggatggacaggtggtttcttcccacaagagagaaatcta

SEQ ID NO: 50 - DNA, 80 bp, Artificial sequence
  chemically synthesized
tgcagactcagtggagaaggagttggaccagtcaacatctctgttgtcacaagcagtgtttcctctggatatggcagtgg

SEQ ID NO: 51 - DNA, 80 bp, Artificial sequence
  chemically synthesized
gagatcaaagactacagtccctacttcaagaccattgaggacctgaggaacaagattctcacagccacagtggacaatgc

SEQ ID NO: 52 - DNA, 80 bp, Artificial sequence
  chemically synthesized
caaagccttggtcaaaaagtatcaggaaggcctcatcacacttacagaacttgctgattctttgctgagccggttagtcc

SEQ ID NO: 53 - DNA, 80 bp, Artificial sequence
  chemically synthesized
cccacctggattctatggagtgaactgtgacaaagcaaactgctcaaccacctgctttaatggagggacctgtttctacc

SEQ ID NO: 54 - DNA, 80 bp, Artificial sequence
  chemically synthesized
aaggagcaggccatgtttgataagaaggtgcagctccagagaatggtagaccaaaggtcggtgatttcagatgaaaagaa

SEQ ID NO: 55 - DNA, 80 bp, Artificial sequence
  chemically synthesized
tagattctagtgcattcaagcacaatggcacggttgaatgtaaggcttacaacgatgtgggcaagacttctgcctatttt

SEQ ID NO: 56 - DNA, 80 bp, Artificial sequence
  chemically synthesized
ctacagtgacttgacaatgaaaaccagcgacaagttcaagtttgtcttccgagaaaagatgggcaggattgttgattatt

SEQ ID NO: 57 - DNA, 80 bp, Artificial sequence
  chemically synthesized
atggcgacaagaagattgtccccaagaagaagaagcccctgaagttggggcccatcaagaagaaggacctgaagaagctt

SEQ ID NO: 58 - DNA, 80 bp, Artificial sequence
  chemically synthesized
gaatttgtacgagacctgcttctatctcctgaagaaaactgtggcattctggaatgggaagatagggaacaaggaatttt

SEQ ID NO: 59 - DNA, 80 bp, Artificial sequence
  chemically synthesized
agcattgtccgagaggaccagaccctgaaacctggcaaagtcgatgtgaagacagacaaatgggatttctactgccagtg

SEQ ID NO: 60 - DNA, 80 bp, Artificial sequence
  chemically synthesized
ccattctttgggtatgggacattcctctgatcctaatgcagtgatgtatccaacctatggaaatggagatccccaaaatt

SEQ ID NO: 61 - DNA, 80 bp, Artificial sequence
  chemically synthesized
tttgtgttcccttacccatatccatttcgcccacttccaccaattccatttccaagatttccatggtttagacgtaattt

SEQ ID NO: 62 - DNA, 80 bp, Artificial sequence
  chemically synthesized
gaggaggagaccgtggacggacgcaagtgcaggagtttagccacttgggagaatgagaacaagatccactgcacccaaac

SEQ ID NO: 63 - DNA, 80 bp, Artificial sequence
  chemically synthesized
cgcacagggaatggattgttggagagagtaactaggattctagcttctctggattttgctcagaacttcatcacaaacaa

SEQ ID NO: 64 - DNA, 80 bp, Artificial sequence
  chemically synthesized
aagatcaaggccataacccaggagaccatcaacggaagattagttctttgtcaagtgaatgaaatccaaaagcacgcatg

SEQ ID NO: 65 - DNA, 80 bp, Artificial sequence
  chemically synthesized
atgcccagaaggatgtggatgctgtggacaaggtgatgaaggagctagacgagaatggagacggggaggtggacttccag

SEQ ID NO: 66 - DNA, 80 bp, Artificial sequence
  chemically synthesized
tgcgaaatggaagcctgcacatcattgatgacttcgagtctaaattcacgttccattctgtggaagactttccccctccg

SEQ ID NO: 67 - DNA, 80 bp, Artificial sequence
  chemically synthesized
ctgctctttgtctacgcagatgattgccttgctcagtgtggcaaagattgcaaatcttactgctgtgatggaaccacgcc

SEQ ID NO: 68 - DNA, 80 bp, Artificial sequence
  chemically synthesized
tgtaccacgccatcttcctggaagagctgaccaccttggagctgattgagaagattgccaacctgtacagcatctccccc

SEQ ID NO: 69 - DNA, 80 bp, Artificial sequence
  chemically synthesized
gttgggcatagcattgctcttttgcatcctgtttacgctggtctgtggggcttctgggacgtctaaacaaccaaaagtaa

SEQ ID NO: 70 - DNA, 80 bp, Artificial sequence
  chemically synthesized
tggcagcagtaagatcttccctggcaactgggacaaccactcccacaagaagaacttgtttgagacgcccatcctggctc

SEQ ID NO: 71 - DNA, 80 bp, Artificial sequence
  chemically synthesized
caacccaatgacccaggagtctacactcaagtgtgcaagttcaccaagtggataaatgacaccatgaaaaagcatcgcta

SEQ ID NO: 72 - DNA, 80 bp, Artificial sequence
  chemically synthesized
cttctgggggtcacagagcatgttctcgccaacaatgatgtttcctgtgaccacccctctaacaccgtgccctctgggag

SEQ ID NO: 73 - DNA, 80 bp, Artificial sequence
  chemically synthesized
ccatcccatagaagtccagcagacaggatttgttaagtgccagactttgtcaggaagtcaaggagcttctgctttgtccg

SEQ ID NO: 74 - DNA, 80 bp, Artificial sequence
  chemically synthesized
gactacgatgacacagacgatgacgaccctttcaaccctcaggaatccaagcgctttgtgcagtggcagtcgtctatctg

SEQ ID NO: 75 - DNA, 80 bp, Artificial sequence
  chemically synthesized
ttcagcgaattgctctgctgcccaaaagacgttttctttggaccaaagatctctttcgtgattccttgcaacaatcaatg

SEQ ID NO: 76 - DNA, 80 bp, Artificial sequence
  chemically synthesized
gggagacaagcacaagctgaagaaatccgaactcaaggagctcatcaacaatgagctttcccatttcttagaggaaatca

SEQ ID NO: 77 - DNA, 80 bp, Artificial sequence
  chemically synthesized
ctcattcaggtgatgtgctctatgaactccttcagcatattctgaagcagaggaaacctcggattcttttttcaccattc

SEQ ID NO: 78 - DNA, 80 bp, Artificial sequence
  chemically synthesized
aagaaatacagagaaggacagtaatgctgagaacatgaattctcaacctgagaaaacagttactactcccacagaaaaaa

SEQ ID NO: 79 - DNA, 80 bp, Artificial sequence
  chemically synthesized
acaataagaacgggttcaaaatgttcgactcctccaactatcatggccaagacctgcttttcaaggatgccaccgtccgg

SEQ ID NO: 80 - DNA, 80 bp, Artificial sequence
  chemically synthesized
agtcctccatcactttgattcttctagtcaagagtcagtgccaaaaaggagaaagtttagtgaaccaaaggaacatatat

SEQ ID NO: 81 - DNA, 80 bp, Artificial sequence
  chemically synthesized
atgtggtccagcaaggaactgcagggtgtcctcagacagcaaggtttcgataggaacatgctgggaaccttagctggggc

SEQ ID NO: 82 - DNA, 80 bp, Artificial sequence
  chemically synthesized
gaacaactctccagtgaacgggaatagtagctgtcaaatggccttcccttccagccagtctctgtaccgcacgtccggag

SEQ ID NO: 83 - DNA, 80 bp, Artificial sequence
  chemically synthesized
tcatgtgctctcatggaacaacagcattatgtcgaccaaacttcagaatgtttctccagcccaagctacttgagaatatt

SEQ ID NO: 84 - DNA, 80 bp, Artificial sequence
  chemically synthesized
agtaccagccttccaaatgaaactctttcagagttagagacacctgggaaatactcacttacaccaccaaaccactgggg

SEQ ID NO: 85 - DNA, 80 bp, Artificial sequence
  chemically synthesized
gcttgaagaccatggtgagaaaaaccagaagcttgaagaccatggtgtgaaaatccttgaacccacccaaatacctgctc

SEQ ID NO: 86 - DNA, 80 bp, Artificial sequence
  chemically synthesized
tctctattcccagtataagggacgaaactgcgaactccttaaagctctatctagccaaaccgcttacgaccttgtatata

SEQ ID NO: 87 - DNA, 80 bp, Artificial sequence
  chemically synthesized
ttccgcttggagcccaaggatgggaaaggtgtgtttgcagtggatggggaattgatggttagcgaggccgtgcagggcca

SEQ ID NO: 88 - DNA, 80 bp, Artificial sequence
  chemically synthesized
gcagacaagttcctgcagctgtttggaaccaaaggtgtcaagagggtgctgtgtcctatcaactaccccttgtcgcccac

SEQ ID NO: 89 - DNA, 80 bp, Artificial sequence
  chemically synthesized
cttggctgctcctctgacaccatcgaggtctcctgcaacttcactcatggtggacagacgtgtctcaagcccatcacggc

SEQ ID NO: 90 - DNA, 80 bp, Artificial sequence
  chemically synthesized
cccacgcatgagatccgctgcaactctgaggtcaagtacgcctcggagaagcatttccaggacaaggtcttctatgcgcc

SEQ ID NO: 91 - DNA, 80 bp, Artificial sequence
  chemically synthesized
gtccttcgatgaggtcaagcgccttcagaagcagatcctggaggtcaagtcctgcctgaagaacaagaccaaggaggcgg

SEQ ID NO: 92 - DNA, 80 bp, Artificial sequence
  chemically synthesized
aaaccagcaaccatcaaggtctatgactactacctaccagatgaacaggcaacaattcagtattctgatccctgtgaatg

SEQ ID NO: 93 - DNA, 80 bp, Artificial sequence
  chemically synthesized
ggacaatttgcgatgacgagtggcaaaattctgatgccattgtcttctgccgcatgctgggttactccaaaggaagggcc

SEQ ID NO: 94 - DNA, 80 bp, Artificial sequence
  chemically synthesized
tcaacgaagaattgtggcccaatcccttgcagttctaccttttgagtgaaggggctcgtgtagagaaaggaaaggaaaaa

SEQ ID NO: 95 - DNA, 80 bp, Artificial sequence
  chemically synthesized
gatgctttcttctgtattggctgctctcattggaattgcaggatctggctactgtgtcattgtggcagcccttggcttag

SEQ ID NO: 96 - DNA, 80 bp, Artificial sequence
  chemically synthesized
acaataacaagaaaattgaaagatgggaaattagtggtggagtgtgtcatgaacaatgtcacctgtactcggatctatga

SEQ ID NO: 97 - DNA, 80 bp, Artificial sequence
  chemically synthesized
cttcagctgtctgtacccagatggcgtcttctatgacctggacagctgcaagcattccagctaccctgattcagaggggg

SEQ ID NO: 98 - DNA, 80 bp, Artificial sequence
  chemically synthesized
tgccagaacactattcaaccaagtgatggaaaaggagtttgaagacggcatcattaactggggaagaattgtaaccatat

SEQ ID NO: 99 - DNA, 80 bp, Artificial sequence
  chemically synthesized
ctcccggaactggcaggactacggagttcgagaagtggaccaagtgaaacgtctcacaggcccaggacttagcgaggggc

SEQ ID NO: 100 - DNA, 80 bp, Artificial sequence
  chemically synthesized
aaggacgtcaacatcttcagctttctttccaagaaggaagagacctacaacgacctcatcaagcagatcgggaagaaggc

SEQ ID NO: 101 - DNA, 80 bp, Artificial sequence
  chemically synthesized
acatgatagacctacagctctcatcaatggccatttgaattcacttcagagcaatccactactgccagaccagaaggtct

SEQ ID NO: 102 - DNA, 80 bp, Artificial sequence
  chemically synthesized
tctaagtttcttttccttttctgtctgtatctgtttttctctgactgcctatatcttactttgtatacccatacataaat

SEQ ID NO: 103 - DNA, 80 bp, Artificial sequence
  chemically synthesized
gtcatttatcctgcagtgctttgctgcaagataacatcgctgatgctgtagcttgtgcaaagagggttgtccgtgatcca

SEQ ID NO: 104 - DNA, 80 bp, Artificial sequence
  chemically synthesized
catggtggagctgctgctgctgcagaacgcacaggtgcaccagttggtcctgcagaactggatgctcaaggccctgcccc

SEQ ID NO: 105 - DNA, 80 bp, Artificial sequence
  chemically synthesized
cttctctatgtgaagcagcagccttggtattgtgaggtctaccaatacagtgagtgttttctggccaaccaaagcccata

SEQ ID NO: 106 - DNA, 80 bp, Artificial sequence
  chemically synthesized
accgtggtgccagtgcttgctggtgtaggcccactggatccccagagcctcaggcccaactcggaggaggtgagctgggg

SEQ ID NO: 107 - DNA, 80 bp, Artificial sequence
  chemically synthesized
gactgactacaaacaatgcatggccgtgaacttctttggaactgtggaggtcacaaagacgtttttgcctcttcttagaa

SEQ ID NO: 108 - DNA, 80 bp, Artificial sequence
  chemically synthesized
aagtcctatgaaactgctgataccagcagccaaggttccactataagtgttgactcacaagatgaccagatggagtctta

SEQ ID NO: 109 - DNA, 80 bp, Artificial sequence
  chemically synthesized
aacactgatgttcccatcccaacacactactttgtggtgctgaccagttgtaaaaacaagagccacacaccggaaaactg

SEQ ID NO: 110 - DNA, 80 bp, Artificial sequence
  chemically synthesized
gccactgcaagttccactggtgctgctacgtcaagtgcaagaagtgcacggagatcgtggaccagtttgtgtgcaagtag

SEQ ID NO: 111 - DNA, 80 bp, Artificial sequence
  chemically synthesized
ggtgcctacagccagctcaaagtggtcttagagaagctgagcaaggacactcactgggtacctgttagttgggtcaggta

SEQ ID NO: 112 - DNA, 80 bp, Artificial sequence
  chemically synthesized
aatcgtggatggcagattactgtaaagatgacaaggacatagagtcagctaaatcagaagactggatgggctcttcggtg

SEQ ID NO: 113 - DNA, 80 bp, Artificial sequence
  chemically synthesized
acctacctctatgaggacagcaatgtatggcacgacatctcagacttcctcgtctataacaagagccgtccctccaatta

SEQ ID NO: 114 - DNA, 80 bp, Artificial sequence
  chemically synthesized
ccggtcagcgggatcctgttcaataatgaaatggacgacttcagctctcccagcatcaccaacgagtttggggtaccccc

SEQ ID NO: 115 - DNA, 80 bp, Artificial sequence
  chemically synthesized
agcatgaacgccaaggaatgtacgttgagaatcactgctccaggcctgcattactccttcagctctggggcagaggaagc

SEQ ID NO: 116 - DNA, 80 bp, Artificial sequence
  chemically synthesized
aacatccaggataaggaccggatctctgccatgcagagcatcttccagaagaccaggactctgggaggcgaggagagctg

SEQ ID NO: 117 - DNA, 80 bp, Artificial sequence
  chemically synthesized
ccttcccatcgcacaacccaaaaaagttatcacaccggaaagaagtcaccgagcgtctactccagaagtcagtatgtgta

SEQ ID NO: 118 - DNA, 80 bp, Artificial sequence
  chemically synthesized
aactattacatccttaacaccctctaccccaagttcaatgataagttggccgaaggcttcccccttcctctgctgaagcg

SEQ ID NO: 119 - DNA, 80 bp, Artificial sequence
  chemically synthesized
ggagactggtttgaatatgtttcttcccctaactacttagcagagctgatgatctacgtttccatggccgtcacctttgg

SEQ ID NO: 120 - DNA, 80 bp, Artificial sequence
  chemically synthesized
attccgagtggctgcctacaacaaagccaaaaaagccatggacttctatgatccagcaaggcacaatgagtttgacttca

SEQ ID NO: 121 - DNA, 80 bp, Artificial sequence
  chemically synthesized
actattctcttgtttactgccttttgactcggatgaagagacacggaaggggagaaatcattggaattcagaagctgaat

SEQ ID NO: 122 - DNA, 80 bp, Artificial sequence
  chemically synthesized
aaagcttatggctctgtgatgatattagtgaccagcggagatgataagcttcttggcaattgcttacccactgtgctcag

SEQ ID NO: 123 - DNA, 80 bp, Artificial sequence
  chemically synthesized
tggtcttgagagatggcttgctggagaaccagaccccagagttcttccaggacgtctgcaagcccaagtacagcggcacc

SEQ ID NO: 124 - DNA, 80 bp, Artificial sequence
  chemically synthesized
gttgaagatgaaacagtagagcttgatgtgtcagatgaagagatggctagaagatatgagaccttggtggggacaattgg

SEQ ID NO: 125 - DNA, 80 bp, Artificial sequence
  chemically synthesized
ctgtgccaagcccttcaaagtcatctgtatcttcgtctctttcctcagctttgactacaaactggtgcagaaggtgtgcc

SEQ ID NO: 126 - DNA, 80 bp, Artificial sequence
  chemically synthesized
ctaatcttatgaacagtggtgtgagactgaatgccatttgtccaggctttgttaacacagccatccttgaatcaattgaa

SEQ ID NO: 127 - DNA, 80 bp, Artificial sequence
  chemically synthesized
ttggattacaggagatgagagtattgtaggccttatgaaggacattgtaggagccaatgagaaagaaatagccctaatga

SEQ ID NO: 128 - DNA, 80 bp, Artificial sequence
  chemically synthesized
caggcactgaacaatttggggtttaagatttgtccctgtggctggcatcagtggaaatgcacccccaagaaatgttgttg

SEQ ID NO: 129 - DNA, 80 bp, Artificial sequence
  chemically synthesized
catgtcaccacgatctttcctctgcttgagaagaccatggaactggatagctcacttccggaatacaacatgtttccccg

SEQ ID NO: 130 - DNA, 80 bp, Artificial sequence
  chemically synthesized
tgttcgagccgcattctggagaagactacagtcgtgatgaggaccacatcgctcacatagtggagcttctgggggacatc

SEQ ID NO: 131 - DNA, 80 bp, Artificial sequence
  chemically synthesized
catcacatcctcatgcacctcaccgagaaagcccaggaggtgacaaggaaataccaggaaatgacgggacaagtttggta

SEQ ID NO: 132 - DNA, 80 bp, Artificial sequence
  chemically synthesized
tcgctgctgtgtcactcatgactgttgctacaaacgtctggagaaacgtggatgtggcaccaaatttctgagctacaagt

SEQ ID NO: 133 - DNA, 80 bp, Artificial sequence
  chemically synthesized
cctgcactcccgaaagaatatgctgttggccaccaagcaactttgtaaagaatttacggatctactggcgcaggaccgga

SEQ ID NO: 134 - DNA, 80 bp, Artificial sequence
  chemically synthesized
gcacattcaagaacacggagattagtttccagctgggagaagagtttgatgaaaccactgcagatgatagaaactgtaag

SEQ ID NO: 135 - DNA, 80 bp, Artificial sequence
  chemically synthesized
gtccttcaaagctggagtctgtcctcctaagaaatctgcccagtgccttagatacaagaaacctgagtgccagagtgact

SEQ ID NO: 136 - DNA, 80 bp, Artificial sequence
  chemically synthesized
atgaaatggagaacttgctgacctacaagcggagagccatagagcacgtgctgcaggtagaggcctcccaggagccctcg

SEQ ID NO: 137 - DNA, 80 bp, Artificial sequence
  chemically synthesized
atggaggacctggacacaaatgcagacaagcagctgagcttcgaggagttcatcatgctgatggcgaggctaacctgggc

SEQ ID NO: 138 - DNA, 80 bp, Artificial sequence
  chemically synthesized
agcttctccagcagtgcgggtcctgggctcctgaaggcttattccatccggaccgcatccgccagtcgcaggagtgcccg

SEQ ID NO: 139 - DNA, 80 bp, Artificial sequence
  chemically synthesized
agtggtgcactcttctttatcatctcagacctgaccatcgccctcaacaaattctgttttcctgtgccctactctcgggc

SEQ ID NO: 140 - DNA, 80 bp, Artificial sequence
  chemically synthesized
ctgtctcttacacggtagcaccctttgtgatgttggcagttgaaccgaccatcagcttatacaagtccatgtacttttat

SEQ ID NO: 141 - DNA, 80 bp, Artificial sequence
  chemically synthesized
tcctgtgctgctgtctggttgagagcctgccaccgtgtgtcgggagtgtgggccaggctgagtgcataggtgacagggcc

SEQ ID NO: 142 - DNA, 80 bp, Artificial sequence
  chemically synthesized
cctgctctggatcatcgaactgaataccaacacaggcatccgtaagaacttggagcaggagatcatccagtacaacttta

SEQ ID NO: 143 - DNA, 80 bp, Artificial sequence
  chemically synthesized
tgatggggagaatgtgaaaattccagtggccatcaaagtgttgagggaaaacacatcccccaaagccaacaaagaaatct

SEQ ID NO: 144 - DNA, 80 bp, Artificial sequence
  chemically synthesized
attgaggccatccgaagagccagtaatggagaaaccctagaaaagatcaccaacagccgtcctccctgcgtcatcctgtg

SEQ ID NO: 145 - DNA, 80 bp, Artificial sequence
  chemically synthesized
ctcgatgcacacactggtatatcccatgaagacctcatccagaacttcctgaatgctggcagctttcctgagatccaggg

SEQ ID NO: 146 - DNA, 80 bp, Artificial sequence
  chemically synthesized
cctgacatggactatgaccctgaggcacgaattctctgtgcgctgtatgttgttgtctctatcctgctggagctggctga

SEQ ID NO: 147 - DNA, 80 bp, Artificial sequence
  chemically synthesized
tcgtgctgtacttcctcaccagcttctacactaagtacgaccagatccattttgtgctcaacaccgtgtccctgatgagc

SEQ ID NO: 148 - DNA, 80 bp, Artificial sequence
  chemically synthesized
acgatgtgcagccttcgaagttgatgcgactgctgagctctaatgaggacgatgccaacatcctttcgagccccacagac

SEQ ID NO: 149 - DNA, 80 bp, Artificial sequence
  chemically synthesized
tgtatcacatcacttctcaagtattccttcattgggcttcatccttttagcagaactcttggtggtgggatagagactta

SEQ ID NO: 150 - DNA, 80 bp, Artificial sequence
  chemically synthesized
atgaagatcggaagaatccagcatacatacctcggaaagggctcttctttgagcatgatcttcgagggcaaactcaggag

SEQ ID NO: 151 - DNA, 80 bp, Artificial sequence
  chemically synthesized
tgtccggtctttcttggatgattttgagtcaaagtattccttccatccagtagaagactttcctgctccagaagaatata

SEQ ID NO: 152 - DNA, 80 bp, Artificial sequence
  chemically synthesized
acatgactctaagtaacctctgtgaccctcagttgaaaccagaagagagagaatcttgtaaccctcaggactgtgtccct

SEQ ID NO: 153 - DNA, 80 bp, Artificial sequence
  chemically synthesized
accttcctctcctaaacacgtctcttctgtcacttcccgaactggcagttctggagcaaaggagatgaaactcaaggggg

SEQ ID NO: 154 - DNA, 80 bp, Artificial sequence
  chemically synthesized
tgctggctcggttatcagactctcatccaacacaagctcacagggaaagccgttccttgctccttgtggagggagctacc

SEQ ID NO: 155 - DNA, 80 bp, Artificial sequence
  chemically synthesized
gcatctgagcagtgaggagatgatgagagaatgtggagatgtgtcaagggctcaagaggagctaaggaaaaaacagaaaa

SEQ ID NO: 156 - DNA, 80 bp, Artificial sequence
  chemically synthesized
aattcttcgaggacattttacagccagagacagagtttgtctttcctctgtcccatctgcatctcgagtcgcagagaccc

SEQ ID NO: 157 - DNA, 80 bp, Artificial sequence
  chemically synthesized
ccaggtgcaggatatcgtcaagccaaacacagcacatacattcatcataacaggaagaaaaaggtccctggagctgcaga

SEQ ID NO: 158 - DNA, 80 bp, Artificial sequence
  chemically synthesized
gtctcggtgcttccagccacataaccacaaaatcaatggctgctcctccaagctgtggtctgagcccactgctggtcctg

SEQ ID NO: 159 - DNA, 80 bp, Artificial sequence
  chemically synthesized
ctgggcactatggaggtgaagagcaagtttggagctgaatttcgtcggttttcgctggaaagatcaaaacctggaaaatt

SEQ ID NO: 160 - DNA, 80 bp, Artificial sequence
  chemically synthesized
gtgcaggttcagtgtgagcagaactggggaagcctgtgctccatcttgagcttctgcacctcggccatccagaagcctcc

SEQ ID NO: 161 - DNA, 80 bp, Artificial sequence
  chemically synthesized
ctgttgctgtgttctgtcatgagttgcctcatgaattaggtgactttgctgttctactaaaggctggcatgaccgttaag

SEQ ID NO: 162 - DNA, 80 bp, Artificial sequence
  chemically synthesized
caatatcaccgccatgccacacaatggatcattctggaatgtccaggatctgctcaattcagcaatgccaagggtggtcc

SEQ ID NO: 163 - DNA, 80 bp, Artificial sequence
  chemically synthesized
tcgctgtccttgcggaatccacacactttgggcctaagccggtaccacccctatggcaagagccacttatccacagcggg

SEQ ID NO: 164 - DNA, 80 bp, Artificial sequence
  chemically synthesized
gcaagtaaagaagaggcaaccacgttctccaatgcaatgctgtttgccctgaacatcatgaattcccaagaaggaggccc

SEQ ID NO: 165 - DNA, 80 bp, Artificial sequence
  chemically synthesized
ggtgcagatgatggctttgttacggtgagcctaaagccagacaaagggaagagagcaaattcccaagaaaatcgtaatta

SEQ ID NO: 166 - DNA, 80 bp, Artificial sequence
  chemically synthesized
cgctgaaacacgtccatttggagaacaaccgcttgaaccagctaccctcgaacttccccttcgacagcctggagaccctc

SEQ ID NO: 167 - DNA, 80 bp, Artificial sequence
  chemically synthesized
tggcctttcattgtgatctctgatgactcctgcgtgatgtggaacgtggtggatgtcaactctgctggggagagaagcag

SEQ ID NO: 168 - DNA, 80 bp, Artificial sequence
  chemically synthesized
ataatcagcaatgatgtctgcaatggcgctgacttctatggaaaccagatcaagcccaagatgttctgtgctggctaccc

SEQ ID NO: 169 - DNA, 80 bp, Artificial sequence
  chemically synthesized
cggtgatggtattttgcccaggcaacagtacttcaaacagaactgcagtcagcatgaatccagtccagatatttcacatt

SEQ ID NO: 170 - DNA, 80 bp, Artificial sequence
  chemically synthesized
ccagcttctgcagtttctgttttgaagagccagcacttacggcagaaacttcttcagtctctgtttcttgataaagtgta

SEQ ID NO: 171 - DNA, 80 bp, Artificial sequence
  chemically synthesized
cacaggattcctgtgggtccagctttggaactgggaaacctttcttcggatccgcactcattccactgatgccagctgcc

SEQ ID NO: 172 - DNA, 80 bp, Artificial sequence
  chemically synthesized
tctacattctggttgtcatgtccttctacggcattttcttgatcggaatcatgctgggctacatgaaatccaagaggcgg

SEQ ID NO: 173 - DNA, 80 bp, Artificial sequence
  chemically synthesized
tggtggagatcttcgacatgctgctggctacatcatctcggttccgcatgatgaatctgcagggagaggagtttgtgtgc

SEQ ID NO: 174 - DNA, 80 bp, Artificial sequence
  chemically synthesized
atgaagaatttcttcattctgatctcctagaagacagcaaataccgaaaaatctactcctttactcttaagcctcgaaca

SEQ ID NO: 175 - DNA, 80 bp, Artificial sequence
  chemically synthesized
ctgagcactgctctccgtgacccacactactttgaaaaaccagacgccttcaatcctgaccactttctggatgccaatgg

SEQ ID NO: 176 - DNA, 80 bp, Artificial sequence
  chemically synthesized
aaaatgggagttttctgttccatttcatcaggctctgaatggcttccatcctgcaccactctgtaccataagaacattaa

SEQ ID NO: 177 - DNA, 80 bp, Artificial sequence
  chemically synthesized
tttgggctcttcgctggccttggtggagctggtgccagcaccagtggcagctctggtgcctgtggtttctcctacaagtg

SEQ ID NO: 178 - DNA, 80 bp, Artificial sequence
  chemically synthesized
accagacggaaaacccgtttctgtgcacagtggttgccatcctcctccactacatctacatgagcacctttgcctggacc

SEQ ID NO: 179 - DNA, 80 bp, Artificial sequence
  chemically synthesized
tggagacttcatcctgggctactatagtcacaaccacagcatcctcatcggcatcactgaacccttccagatctcgctgc

SEQ ID NO: 180 - DNA, 80 bp, Artificial sequence
  chemically synthesized
tgagcagcaagcgaatcaccatcaacaagattctgtccaacgagagccttgtgcaggagaacctgtacttccagcgctgc

SEQ ID NO: 181 - DNA, 80 bp, Artificial sequence
  chemically synthesized
ccctctggatgagtccgagagagatggaggctctgaggaccaagtggaagacccagcactaagtgagcctggggaggaac

SEQ ID NO: 182 - DNA, 80 bp, Artificial sequence
  chemically synthesized
aaggcaatggctgtgccctatcttctgagaagaaagttcagtaattccctgaaaagtcaaggtaaagatgatgattcttt

SEQ ID NO: 183 - DNA, 80 bp, Artificial sequence
  chemically synthesized
ctcggagcagcagcataagctggacttcaaggcatacgaacaggcactgcaatactcgccttacggctctacgttgcccg

SEQ ID NO: 184 - DNA, 80 bp, Artificial sequence
  chemically synthesized
cagattcagagtctgatatcctgttgggcattctggacaacttggacccagtcatgttcttcaaatgcccttccccagag

SEQ ID NO: 185 - DNA, 80 bp, Artificial sequence
  chemically synthesized
aatgtctagcaaatccaaaaagtgcaaaaaagtgcatgactcactggaggacttccccaagaacagctcgtttaacccgg

SEQ ID NO: 186 - DNA, 80 bp, Artificial sequence
  chemically synthesized
gggcatctatggccagtatcaactccttggcatcaagagcattggctttgaatggaattatccactagaggagccgacca

SEQ ID NO: 187 - DNA, 80 bp, Artificial sequence
  chemically synthesized
tgggcaagtatattctgctcatcaaatggtcattggctatgtactttgtgcagggaagtacattatctacagtcacaaaa

SEQ ID NO: 188 - DNA, 80 bp, Artificial sequence
  chemically synthesized
cctttactgcccctgcttaacacggtcaagttcctgctgcttttctacctgaagaagcttaccctcttctccacctgctc

SEQ ID NO: 189 - DNA, 80 bp, Artificial sequence
  chemically synthesized
tatgcttacgaacctgcagatacagctctgttgcttgacaacatgaagaaagctctcaagttgctgaagactgaattgta

SEQ ID NO: 190 - DNA, 80 bp, Artificial sequence
  chemically synthesized
ccaggatccctggagtgccttggtgtttcaagcccctgactaggaagacagaatgcaccttctgaggcacctccagctgc

SEQ ID NO: 191 - DNA, 80 bp, Artificial sequence
  chemically synthesized
tgccatagatgaattgaaggaatgttttcttaaccaaacggatgaaactctgagcaatgttgaggtgtttatgcaattaa

SEQ ID NO: 192 - DNA, 80 bp, Artificial sequence
  chemically synthesized
gcttctaccactgctcgtaaagacattccagttttacccaaatgggttggggatctcccgaatggtagagtgtgtccctg

SEQ ID NO: 193 - DNA, 80 bp, Artificial sequence
  chemically synthesized
acctgttcaaggtgcagcgatactgtgagaagagcatgatcagcagaaagctctttggattcactgaacggtacggagca

SEQ ID NO: 194 - DNA, 80 bp, Artificial sequence
  chemically synthesized
cagctcatcactgttgaaaaggctttggcaattctttctcagcctacaccctcacttgttgtggatcatgagcgattaaa

SEQ ID NO: 195 - DNA, 80 bp, Artificial sequence
  chemically synthesized
catccagagaaagcaccttatgagtggaagaatttggatggcatttcccatgcacctaatgctgtgaaaaccgcatttta

SEQ ID NO: 196 - DNA, 80 bp, Artificial sequence
  chemically synthesized
accctgagactcccagatcttcaggttctatgcgcaatagatggaaaccaaacagcagcaaggtactagggagatccccc

SEQ ID NO: 197 - DNA, 80 bp, Artificial sequence
  chemically synthesized
agcactctacacagtcacgggacaaagctggcctgaatcattaatacgaaagactggatataccctggaaagtcttaagc

SEQ ID NO: 198 - DNA, 80 bp, Artificial sequence
  chemically synthesized
gaataagtatgcaagcagcaaactcctgaagatcagcatgatccctcagctgaactcaaaagccgtcaaagaccttgcct

SEQ ID NO: 199 - DNA, 80 bp, Artificial sequence
  chemically synthesized
ctctgtgttaccaagaactggctgtctgcagatactaaagaagagcgggatctctggatgcaaaaactcaatcaagttct

SEQ ID NO: 200 - DNA, 80 bp, Artificial sequence
  chemically synthesized
gctgccgagctctggaaaaaccccacagcttttaagaagtacctgcaagaaacctactcaaagcaggtcaccagccagga

SEQ ID NO: 201 - DNA, 80 bp, Artificial sequence
  chemically synthesized
tgggttcattacatgattcatgagccagaaccacatattcttctctttagacgacctcttccaaaagatcaacaaaaatg

SEQ ID NO: 202 - DNA, 80 bp, Artificial sequence
  chemically synthesized
agcctgcagcaagcactttggagagcaaatctgtgcagagagtaacgcggagtgtcaagaggtgtgcagaaaatccaaag

SEQ ID NO: 203 - DNA, 80 bp, Artificial sequence
  chemically synthesized
gaaggcctggtcctggacacaatgaatgacagcctcagcaagatcctgctggacatcagctttcctggcctggacgagga

SEQ ID NO: 204 - DNA, 80 bp, Artificial sequence
  chemically synthesized
gacagagaagcatgcaagacagaaacaaaaggctgatgaggaagagatgcttgataatctaccagaggctggtgactcca

SEQ ID NO: 205 - DNA, 80 bp, Artificial sequence
  chemically synthesized
gaatggtttctgcataccctcgacagctagagtcattaatccgcttagcagaagcccatgctaaagtaagattgtctaac

SEQ ID NO: 206 - DNA, 80 bp, Artificial sequence
  chemically synthesized
taagagatcattatccttgtgtgtcttcattggaacagctaacggagttgggaagagaaactgatgcttttgtatgccgc

SEQ ID NO: 207 - DNA, 80 bp, Artificial sequence
  chemically synthesized
ggactgcagagaaggaactcctctgaagcaagctccggagacttcttagatctgaagggagaaggtgatattcatgaaaa

SEQ ID NO: 208 - DNA, 80 bp, Artificial sequence
  chemically synthesized
ctgtagaggaagaggaagatcaaaacgttgtaccagaaactacctctgaaggctacactttccaagttcaggatggggct

SEQ ID NO: 209 - DNA, 80 bp, Artificial sequence
  chemically synthesized
ggcttctgaactggaattctgcagctaacccttccacgactagaaccttaggcattggggagttttagatggactaattt

SEQ ID NO: 210 - DNA, 80 bp, Artificial sequence
  chemically synthesized
atctcaggactctaaatactctaacagctgtccagtgttggtgattccagtggactgtatctctgtgaaaaacacaattt

SEQ ID NO: 211 - DNA, 80 bp, Artificial sequence
  chemically synthesized
catttggagtataggcaccatatttgctgaactagcaactaagaaaccacttttccatggggattcagaaattgatcaac

SEQ ID NO: 212 - DNA, 80 bp, Artificial sequence
  chemically synthesized
tccaatctgttgcagtctccttcaagcattctgtcgaccctggatgttgaattgccacctgtttgctgtgacatagatat

SEQ ID NO: 213 - DNA, 80 bp, Artificial sequence
  chemically synthesized
cgcccaccaagaaggaacatcagaaagcctgggctttgaacctgaacggttttgatgtagaggaagccaagatccttcgg

SEQ ID NO: 214 - DNA, 80 bp, Artificial sequence
  chemically synthesized
gaggaagaccaaaaagtcatcgagctggttaagaagtatggcacaaagcagtggacactgattgccaagcacctgaaggg

SEQ ID NO: 215 - DNA, 80 bp, Artificial sequence
  chemically synthesized
tttgtggcagacagacttatgctggaactgggttttagcaaggttttcagagtagagaacccatttgactttatggagaa